(19) United States
(12) Patent Application Publication
(10) Pub. No.: US 2026/0366025 A1
(43) Pub. Date: Nov. 19, 2026

(54) TARGETED COMPOSITIONS

(71) Applicant: GENEVANT SCIENCES GMBH, Basel (CH)

(72) Inventors: Richard James HOLLAND, Vancouver, British Columbia (CA); Mark Christopher WOOD, Vancouver, British Columbia (CA); Xin YE, Vancouver, British Columbia (CA); Christopher Justin PASETKA, Vancouver, British Columbia (CA); Lorne Ralph PALMER, Vancouver, British Columbia (CA); Sean MONAHAN, Vancouver, British Columbia (CA)

(21) Appl. No.: 19/110,699

(22) PCT Filed: Sep. 11, 2023
(86) PCT No.: PCT/IB2023/059008

§ 371 Date: Mar. 11, 2025

Related U.S. Application Data
(60) Provisional application No. 63/405,697, filed on Sep. 12, 2022.

(51) Int. Cl. C12N 15/113; A61P 1/16

(52) U.S. Cl. CPC C12N 15/1137; A61P 1/16; C12N 15/113; C12Y 502/01008; C12N 2310/14; C12N 2310/3513

(57) ABSTRACT

The invention provides conjugates for the targeted delivery of therapeutic agents to PDGFR expressing cells for the treatment of a disease involving these cells (e.g., liver fibrosis, non-alcoholic steatohepatitis (NASH), clear cell renal cell carcinoma, kidney fibrosis, or alcoholic steatohepatitis (ASH)).

### PRIORITY

This application claims priority to U.S. Provisional Application No. 63/405,697 that was filed on Sep. 12, 2022. The entire content of the applications referenced above is hereby incorporated by reference herein.

### BACKGROUND

Liver fibrosis is caused by excessive accumulation of extracellular matrix during chronic liver injuries. Activation of hepatic stellate cells (HSCs) is a key step during liver fibrogenesis. Targeted delivery of therapeutic agents to HSCs, e.g., activated HSCs, may be important for the successful treatment of diseases of the liver and/or diseases of the kidney (e.g., liver fibrosis, non-alcoholic steatohepatitis (NASH), kidney fibrosis, or alcoholic steatohepatitis (ASH)). Accordingly, means of delivering therapeutics to HSCs are needed.

### BRIEF SUMMARY

The invention provides compounds, compositions and methods that can be used to target oligonucleotides to platelet derived growth factor receptor (PDGFR) expressing cells.

In one aspect this invention provides a compound of formula (I)

(R¹)ₓ-L-(R²)_{y} (I)

or a salt thereof, wherein:
x is 2, 3, 4 or 5;
each R¹ is independently a targeting ligand, which is selected from:
a) a cyclic polypeptide depicted by SEQ ID NO: 1:C*SRNLIDC* (SEQ ID NO: 1), wherein * denotes a disulfide bond linking the two C residues to thereby form the cyclic polypeptide, and
b) any cyclic polypeptide having at least 80% sequence identity (e.g., at least 85%; e.g., at least 87.5%; e.g., at least 90%; e.g., at least 95%; e.g., at least 99% sequence identity) with the polypeptide depicted by SEQ ID NO: 1, under the proviso that both C* residues are present, and wherein * denotes a disulfide bond linking the two C residues to thereby form the cyclic polypeptide;
L is a linking group;
y is 1, 2, 3, 4 or 5;
and each R² is independently an oligonucleotide, a label (e.g., a label derivable from fluorescein isothiocyanate (FITC) or Cy5), a phenyl group that is substituted with a formyl (—CHO) group, or a group of formula:

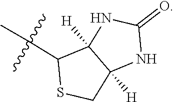 [SMILES: *C1SC[C@@H]2NC(=O)N[C@H]12]

In another aspect, the invention provides a process for preparation of a diblock polymer of the following formula (X):

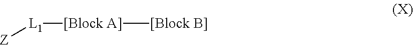

comprising:
a) contacting a compound of structure Va, Vb, Vc, or Vd

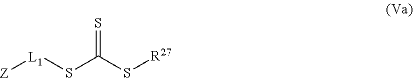

wherein R²⁷ is (C₁-C₁₂)alkyl,

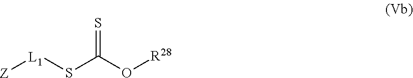

wherein R²⁸ is (C₁-C₁₂)alkyl,

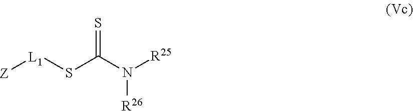

wherein R²⁵ and R²⁶ are independently H, (C₁-C₁₂)alkyl, aryl, or heteroaryl,

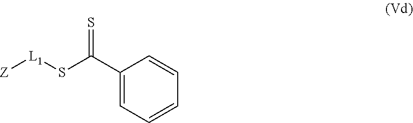

with one or more A monomers selected from the group consisting of:
   a polyethyleneglycol methacrylate with 2-20 ethylene glycol units (PEGMA);
   a (C₄-C₁₈)alkyl-methacrylate;
   a (C₄-C₁₈)branched alkyl-methacrylate;
   a cholesteryl methacrylate;
   a (C₄-C₁₈)alkyl-methacrylate substituted with one or more fluorine atoms; and
   a (C₄-C₁₈)branched alkyl-methacrylate substituted with one or more fluorine atoms;
in the presence of a free radical to provide a first product;
b) contacting the first product with one or more B monomers of formulae B1, B2 and B3

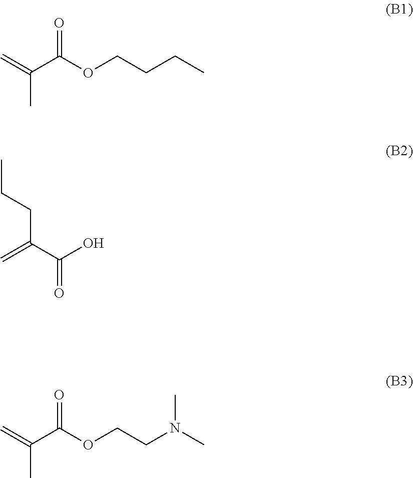

in the presence of a free radical to provide a second product, and
c) optionally contacting the second product with a free radical source (e.g., AIBN) to remove the chain transfer agent and provide the diblock polymer of formula (X);
wherein:
   Block A comprises one or more residues of A monomers and has a molecular weight of from about 1 kDa to about 25 kDa;
   Block B comprises one or more residues of monomers B1, B2, and B3 and has a molecular weight of from about 1 kDa to about 25 kDa.
   L₁ is a linking moiety;
   Z is a functional group that is optionally protected with a protecting group and is capable of reacting with Y of a compound of formula (XII):

T-L₂-Y (XII)
to form a conjugate wherein:
   T is a ligand (optionally targeting ligand);
   Y is a functional group that is capable of reacting with Z to form a conjugate; and
   L₂ is absent or is a linking moiety.

In another aspect, the invention provides a compound of formula (I)

(R¹)ₓ-L-(R²)_{y} (I)
or a salt thereof, wherein:
   x is 1, 2, 3, 4 or 5;
   each R¹ is independently a targeting ligand, which is selected from
   a) a cyclic polypeptide depicted by SEQ ID NO: 1:C*SRNLIDC* (SEQ ID NO: 1), wherein * denotes a disulfide bond linking the two C residues to thereby form the cyclic polypeptide,
   b) any cyclic polypeptide having at least 80% sequence identity (e.g., at least 85%; e.g., at least 87.5%; e.g., at least 90%; e.g., at least 95%; e.g., at least 99% sequence identity) with the polypeptide depicted by SEQ ID NO: 1, under the proviso that both C* residues are present, and wherein * denotes a disulfide bond linking the two C residues to thereby form the cyclic polypeptide;
   L is a linking group; and
   y is 1, 2, 3, 4 or 5;
   and each R² is independently an endosomal release polymer.

The invention also provides synthetic intermediates and methods disclosed herein that are useful to prepare compounds of the formula I and formula XI.

Other objects, features, and advantages of the present invention will be apparent to one of skill in the art from the following detailed description and figures.

### BRIEF DESCRIPTION OF THE FIGURES

FIG. 1. Western blot detection of PDGFRB expression in HSC-T6 and NIH3T3 cells FIG. 2. Uptake of monovalent or trivalent pPB ligand-biotin-AF488-streptavidin complex by HSC-T6 and NIH3T3 cells as analyzed by flow cytometry.

FIG. 3. Uptake of divalent or trivalent pPB ligand-biotin-AF488-streptavidin complex by pHHSCs as analyzed by flow cytometry.

FIG. 4. Western blot detection of PDGFRB expression in LX-2, pHHSC and NIH3T3 cells

FIG. 5. Uptake of trivalent pPB ligand-biotin-AF488-streptavidin complex by LX-2, NIH3T3 and primary human HSC as analyzed by flow cytometry.

FIG. 6. In vitro gene silencing by TripPB siRNA conjugates and chloroquine in NIH3T3 cell.

FIG. 7. In vitro gene silencing by TripPB siRNA conjugates and pPB-ERP in pHHSC.

FIG. 8. In vivo gene silencing by TripPB siRNA conjugates and pPB-ERP in mouse liver fibrosis model.

FIG. 9. Dose response of pPB-ERP when co-treated with TripPB-siRNA conjugate in mouse liver fibrosis model.

FIG. 10. In vivo gene silencing by DipPB and TripPB siRNA conjugates and pPB-ERP in mouse liver fibrosis model.

In the application, including Figures, Examples and Schemes, it is to be understood that an oligonucleotide can be, but is not limited to, a double stranded siRNA molecule.

### DETAILED DESCRIPTION

GalNAc-conjugated short interfering RNA's (siRNA) are a modality for mediating RNA interference (RNAi) in hepatocytes. They comprise two important components; a GalNAc targeting ligand, which binds to the asialoglycoprotein receptor (ASGPr) found on the surface of hepatocytes to mediate uptake, and the siRNA oligonucleotide, that once delivered to the cytoplasm of hepatocytes can mediate destruction of specific mRNA sequences (determined by the sequence of the siRNA) to reduce expression of the associated protein product.

While siRNA delivery to hepatocytes has been demonstrated with this class of therapeutics, successful application to other cell types has been more problematic. Suitable receptors must be identified, which are expressed relatively selectively on the target cell surface and in sufficient numbers. Then, appropriate ligands must be identified that bind with high specificity and affinity to the target receptor. A significant third hurdle is endosomal escape; when bound to a receptor, the ligand conjugate is taken up by the cell and entrapped in an endosome, from which it must escape to reach the cytoplasm. Despite lacking an active endosomal escape mechanism, GalNAc conjugates appear to mediate activity regardless, possibly because the receptor is so abundantly expressed and quickly recycled (~15 minutes) following uptake. The sheer number of conjugate molecules taken up by hepatocytes may obviate the need for an active endosomal escape. This has not been true for other ligand-based siRNA conjugate systems.

Hepatic stellate cells (HSC) play a key role in the progression of fibrosis: In response to chronic liver damage, they become activated, and are largely responsible for collagen deposition and scarring of the liver. A variety of genes are involved and are validated targets for treatment, including RNAi strategies. As described herein, siRNA conjugates have been synthesized to target HSC via platelet-derived growth factor receptor (PDGFR) using a cyclic octapeptide motif. In particular, as described herein, trivalent presentation of a pPB ligand outperforms monovalent presentation for binding and uptake. Gene silencing experiments also demonstrated that trivalent ligand structures are particularly effective, e.g., when compared to mono and divalent conjugates.

The invention also provides pPB conjugates of an endosome release polymer (ERP), the conjugate also being referred to as pPB-ERP. The inventors have found that for the pPB-ERP, monovalency is sufficient, i.e. one ERP moiety being bound to one pPB moiety, optionally via a linker. The terms “endosome release polymer” (ERT) and “diblock polymer” are used interchangeably herein. In particular embodiments, the specific ERT as provided by the present invention, optionally characterized by presence of a functional group Z or alternatively by presence of a moiety T-L2-Y′-X′ as described herein, is obtainable by the process according to the present invention. In particular embodiments, the ERT according to the present invention is conjugated to a specific ligand T, such as e.g. specific targeting ligand, as described herein.

The inventors designed a new process for preparing respectively conjugated ERP, i.e. pPB-ERP. Design of the new process became necessary since previously known processes for preparing ERP, such as with other ligands including N-acetyl glucosamine, proved to be neither resource friendly nor technically applicable to ligands such as pPB. pPB, as well as certain other ligands described herein, such as other polypeptides, whether cyclic or not, are as such not tolerant or inert to RAFT polymerization as used in the prior art. Also, the process according to the present invention is significantly more cost-effective for expensive ligands such as polypeptides, including pPB and derivatives thereof, as a result of the ligand being conjugated in the last step of the process.

The present invention also provides methods for using one or more pPB conjugates of the present invention, such as pPB-siRNA according to the invention, pPB-ERP according to the invention, alone and/or in combination, in the treatment of one or more diseases of a human or an animal, preferably a human.

In certain embodiments, the disease is a liver disease.

In certain embodiments, the disease is a kidney disease.

In certain embodiments, the disease is liver fibrosis.

In certain embodiments, the disease is non-alcoholic steatohepatitis (NASH)

In certain embodiments, the disease is kidney fibrosis.

In certain embodiments, the disease is clear cell renal cell carcinoma.

In certain embodiments, the disease is alcoholic steatohepatitis (ASH)

Also provided is a method of delivering a therapeutic agent to a hepatic stellate cell (HSC), in vivo or in vitro, comprising contacting the HSC with a conjugate as described herein.

Liver fibrosis is caused by excessive accumulation of extracellular matrix during chronic liver injuries. Activation of hepatic stellate cells (HSCs) is a key step during liver fibrogenesis. Targeted delivery of therapeutic agents to HSCs, e.g., activated HSCs, may be important for the successful treatment of liver fibrosis. A number of protein markers have been found to be overexpressed in activated HSCs, and their ligands have been used to specifically deliver various antifibrotic agents. (see, e.g., Chen et al., Journal of Pharmacology and Experimental Therapeutics, 2019, 370 (3) 695-702). However, delivery of therapeutics using other systems is needed as other means for delivering therapeutic agents to HSCs.

Liver fibrosis is caused by the formation of an abnormally large amount of scar tissue in the liver. Liver fibrosis occurs when the liver attempts to repair and replace damaged cells. Various disorders and drugs can damage the liver and cause fibrosis.

Nonalcoholic fatty liver disease (NAFLD) is a condition in which triglycerides accumulate in the liver. Nonalcoholic steatohepatitis (NASH) is a type of NAFLD. NASH is associated with inflammatory changes and liver cell damage. NASH is a leading cause of liver disease and often progresses to liver fibrosis, cirrhosis and hepatocellular carcinoma (HCC). Non-alcoholic steatohepatitis (NASH) and alcoholic steatohepatitis (ASH) have a similar pathogenesis and histopathology but a different etiology and epidemiology. NASH and ASH are advanced stages of non-alcoholic fatty liver disease (NAFLD) and alcoholic fatty liver disease (AFLD). Alcoholic steatohepatitis (ASH) is a chronic, progressive liver disease characterized by fibrosis of the liver as well as possible necrosis of the liver tissue, brought on by excessive, prolonged alcohol use. Women are more susceptible to the disease because alcohol metabolism is lower in women than in men.

Liver fibrosis is an important underlying cause of liver dysfunction and predicts mortality. Progression to cirrhosis and HCC leads to ultimate liver failure and thus liver transplantation is required. The current US prevalence of NASH-related fibrosis (F2 and later) is about 3.8 million patients. Doctors typically recommend weight loss to treat NAFLD and NASH. While weight loss can reduce fat in the liver, inflammation, and fibrosis, no medicines have been approved to treat NAFLD and NASH. Specifically, no medicines have been approved to treat liver fibrosis. (Clin Liver Dis. 2008 November; 12(4):733-46, N Engl J Med. 2017 Nov. 23; 377(21):2063-2072, J Hepatol. 2017 December; 67(6):1265-127) Accordingly, new therapeutic treatment options, including delivery options, are needed for the treatment of liver fibrosis, e.g., in the context of NASH or ASH.

As used herein, the following terms have the meanings ascribed to them unless specified otherwise.

The term “conjugate” as used herein includes compounds of formula (I) that comprise an oligonucleotide (e.g., an siRNA molecule) linked to a targeting ligand. Thus, the terms compound and conjugate may be used herein interchangeably.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and are preferably about 20-24, 21-22, or 21-23 (duplex) nucleotides in length. siRNA duplexes may comprise 3′ overhangs of about 1 to about 5 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

In certain embodiments, the 5′ and/or 3′ overhang on one or both strands of the siRNA comprises 1-5 (e.g., 1, 2, 3, 4 or 5) modified and/or unmodified deoxythymidine (t or dT) nucleotides, 1-5 (e.g., 1, 2, 3, 4 or 5) modified (e.g., 2′OMe) and/or unmodified uridine (U) ribonucleotides, and/or 1-5 (e.g., 1, 2, 3, 4 or 5) modified (e.g., 2′OMe) and/or unmodified ribonucleotides or deoxyribonucleotides having complementarity to the target sequence (e.g., 3′overhang in the antisense strand) or the complementary strand thereof (e.g., 3′ overhang in the sense strand).

Preferably, siRNAs are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNAs are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA of the invention to silence, reduce, or inhibit the expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 8100, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, the synthetic activating group is a group that will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol-1-yloxy)tris(dimethylamino)phosphonium hexafluorophosphate (BOP), benzotriazol-1-yl-oxytripyrrolidinophosphonium hexafluorophosphate (PyBOP) or O-benzotriazol-1-yl-N,N,N′,N′-tetramethyluronium hexafluorophosphate (HBTU).

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “oligonucleotide” includes nucleotides containing up to about 60 nucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), small activating RNA (saRNA), asymmetrical interfering RNA (aiRNA), microRNA (miRNA), tRNA, rRNA, tRNA, viral RNA (vRNA). Accordingly, the term “oligonucleotide” refers to a polymer or oligomer of nucleotide or nucleoside monomers consisting of naturally-occurring bases, sugars and intersugar (backbone) linkages. The term “oligonucleotide” also includes polymers or oligomers comprising non-naturally occurring monomers, or portions thereof. Such modified or substituted oligonucleotides may have properties, for example, of enhanced cellular uptake, reduced immunogenicity and/or increased stability in the presence of nucleases.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

As used herein, the term “label” includes groups that enable the compound to be detected in vitro or in vivo. The term “labelling agent” is used interchangeably with the term label. Without limitation, a label may be selected from the group comprising a fluorophore, a chromophore, and a radionucleotide. In one embodiment, the label can be detected by spectroscopy. In one embodiment, the label can be detected by fluorescence spectroscopy. In one embodiment, the label is a group that is derivable from fluorescein isothiocyanate (FITC) or Cy5. In one embodiment, a label can be detected by an apparatus suitable for detecting any one or more of alpha, beta particles, gamma rays, and x rays.

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane (including straight and branched alkanes), as exemplified by —CH₂CH₂CH₂CH₂— and —CH(CH₃)CH₂CH₂—.

The term “cycloalkyl,” “carbocyclic,” or “carbocycle” refers to hydrocarbon ringsystem having 3 to 20 overall number of ring atoms (e.g., 3-20 membered cycloalkyl is a cycloalkyl with 3 to 20 ring atoms, or C₃₋₂₀ cycloalkyl is a cycloalkyl with 3-20 carbon ring atoms) and for a 3-5 membered cycloalkyl being fully saturated or having no more than one double bond between ring vertices and for a 6 membered cycloalkyl or larger being fully saturated or having no more than two double bonds between ring vertices. As used herein, “cycloalkyl,” “carbocyclic,” or “carbocycle” is also meant to refer to bicyclic, polycyclic and spirocyclic hydrocarbon ring systems, such as, for example, bicyclo[2.2.1]heptane, pinane, bicyclo[2.2.2]octane, adamantane, norbornene, spirocyclic C₅₋₁₂ alkane, etc. As used herein, the terms, “alkenyl,” “alkynyl,” “cycloalkyl,”, “carbocycle,” and “carbocyclic,” are meant to include mono and polyhalogenated variants thereof.

The term “heterocycloalkyl,” “heterocyclic,” or “heterocycle” refers to a saturated or partially unsaturated ring system radical having from 3-20 ring atoms (e.g., 3-20 membered heterocycloalkyl is a heterocycloalkyl radical with 3-20 ring atoms, a C₂₋₁₉ heterocycloalkyl is a heterocycloalkyl having 3-10 ring atoms with between 2-19 ring atoms being carbon) that contain from one to ten heteroatoms selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The terms “halo” or “halogen,” by themselves or as part of another substituent, mean, unless otherwise stated, a fluorine, chlorine, bromine, or iodine atom. The term “(halo)alkyl” is meant to include both a “alkyl” and “haloalkyl” substituent. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means a carbocyclic aromatic group having 6-14 carbon atoms, whether or not fused to one or more groups. Examples of aryl groups include phenyl, naphthyl, biphenyl and the like unless otherwise stated.

The term “heteroaryl” refers to aryl ring(s) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

The term “animal” includes mammalian species, such as a human, mouse, rat, dog, cat, hamster, guinea pig, rabbit, livestock, and the like.

The term “alkylamino” includes a group of formula —N(H)R, wherein R is an alkyl as defined herein.

The term “dialkylamino” includes a group of formula —NR₂, wherein each R is independently an alkyl as defined herein.

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities.

Unless otherwise specifically noted, when a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “peptide” as used herein comprises oligo- and polypeptides and refers to substances comprising two or more, preferably 3 or more, preferably 4 or more, preferably 6 or more, such as 8 or more amino acids joined covalently by peptide bonds. A peptide may include natural amino acids and non-natural amino acids, such as D-amino acids. In one embodiment, a peptide merely includes natural amino acids. In one embodiment, all amino acids in a peptide are L-amino acids. A peptide consisting of amino acids may also be referred to as “polypeptide”, e.g. a polypeptide having 6 to 10 amino acid residues, such as 7 to 9 amino acid residues, and preferably about 8 amino acid residues,

As used herein, a “cyclic peptide” or “cyclized peptide” refers to a peptide or polypeptide chain which forms a ring, preferably to a peptide or polypeptide having an intramolecular bond between two non-adjacent amino acids within a peptide. The intramolecular bond may be selected without limitation from the group consisting of (i) backbone to backbone (i.e. peptide bond), (ii) side-chain to backbone and (iii) side-chain to side-chain cycle formation. A cyclic peptide of the invention may comprise a linking group resulting from any of the foregoing, such as e.g. a peptide bond in case of backbone to backbone cyclization and e.g. disulfide bonds resulting from two residues containing thiol groups, such as cysteines which can form intramolecular disulfide bridges giving cyclic peptides.

As used herein, the term “percentage identity” refers to a percentage of amino acid residues which are identical between the two sequences to be compared, obtained after the best alignment of two sequences over their entire length. Sequence comparisons, for purposes of determining the percentage of sequence identity, between two amino acid sequences are typically carried out by comparing these sequences after having aligned them optimally, said comparison being carried out by segment or by “window of comparison” in order to identify and compare local regions of sequence similarity. The optimal alignment of the sequences for comparison may be produced by means of the local homology algorithm of Smith and Waterman, 1981, Ads App. Math. 2, 482, by means of the local homology algorithm of Needleman and Wunsch, 1970, J. Mol. Biol. 48, 443, by means of the similarity search method of Pearson and Lipman, 1988, Proc. Natl Acad. Sci. USA 85. 2444, or by means of computer programs which use these algorithms (GAP. BESTFIT, FASTA. BLAST P, BLAST N and TFASTA in Wisconsin Genetics Software Package, Genetics Computer Group. 575 Science Drive, Madison, Wis.). Alternatively, two sequences may be aligned manually; this is convenient e.g. for short polypeptide sequences of e.g. 10 amino acid residues or less. The “percentage identity” is calculated as follows:
number of identical positions between the two sequences being aligned and compared,
divided by the number of positions compared,
multiplied the result obtained by 100,
   thereby obtaining the percentage identity between these two sequences.

Unless stated otherwise herein, the term “about”, when used in connection with a value or range of values, means plus or minus 5% of the stated value or range of values.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNAs are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, CA). However, a larger or smaller scale of synthesis is also within the scope of this invention. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

Linking Group (L, L₁ or L₂).

The linking group can be variable provided the targeting conjugate functions as described herein. The linking group can vary in length and atom composition and for example can be branched or non-branched or cyclic or a combination thereof. The linking group may also modulate the properties of the targeted conjugate such as but not limited to solubility, stability and aggregation.

In one embodiment the linker comprises about 3-5000 atoms. In one embodiment the linker comprises about 3-4000 atoms. In one embodiment the linker comprises about 3-2000 atoms. In one embodiment the linker comprises about 3-1000 atoms. In one embodiment the linker comprises about 3-750 atoms. In one embodiment the linker comprises about 3-500 atoms. In one embodiment the linker comprises about 3-250 atoms. In one embodiment the linker comprises about 3-100 atoms. In one embodiment the linker comprises about 3-50 atoms. In one embodiment the linker comprises about 3-25 atoms.

In one embodiment the linker comprises about 10-5000 atoms. In one embodiment the linker comprises about 10-4000 atoms. In one embodiment the linker comprises about 10-2000 atoms. In one embodiment the linker comprises about 10-1000 atoms. In one embodiment the linker comprises about 10-750 atoms. In one embodiment the linker comprises about 10-500 atoms. In one embodiment the linker comprises about 10-250 atoms. In one embodiment the linker comprises about 10-100 atoms. In one embodiment the linker comprises about 10-50 atoms. In one embodiment the linker comprises about 10-25 atoms.

In one embodiment the linker L comprises atoms selected from H, C, N, S and O.

In one embodiment the linker L comprises atoms selected from H, C, N, S, P and O.

In one embodiment the linker L comprises a branched or unbranched, saturated or unsaturated, hydrocarbon chain, having from about 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl. In one embodiment the linker comprises a branched or unbranched, saturated or unsaturated, hydrocarbon chain, having from about 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, wherein each R^{a} is independently H or (C₁-C₆)alkyl.

In one embodiment the linker L comprises a polyethylene glycol. In one embodiment the linker comprises a polyethylene glycol linked to the remainder of the targeted conjugate by a carbonyl group. In one embodiment the polyethylene glycol comprises about 1 to about 500 or about 5 to about 500 or about 3 to about 100 repeat (e.g., —CH₂CH₂O—) units (Greenwald, R. B., et al., Poly (ethylene glycol) Prodrugs: Altered Pharmacokinetics and Pharmacodynamics, Chapter, 2.3.1., 283-338; Filpula, D., et al., Releasable PEGylation of proteins with customized linkers, Advanced Drug Delivery, 60, 2008, 29-49; Zhao, H., et al., Drug Conjugates with Poly(Ethylene Glycol), Drug Delivery in Oncology, 2012, 627-656).

In one embodiment the linker L is —NH(CH₂CH₂O)₄CH₂CH₂C(═O)—. In one embodiment the linker L is —NH(CH₂CH₂O)ₙCH₂CH₂C(═O)— wherein n is 1-500, 5-500, 3-100, 5-50, 1-50, 1-20, 1-10, 1-5, 2-50, 2-20, 2-10, 2-5, 3-50, 3-20, 3-10, 3-5, 4-50, 4-20, 4-10, 4-5.

In one embodiment the linker L is —(CH₂CH₂O)₄CH₂CH₂C(═O)—.

In one embodiment the linker L is a branched or unbranched, saturated or unsaturated, hydrocarbon chain, having from about 1 to about 500 carbon atoms, wherein one or more of the carbon atoms is optionally replaced independently by —O—, —S, —N(R^{a})—, 3-20 membered heterocycle, 3-20 membered heteroaryl or 3-20 membered carbocycle and wherein each chain, 3-20 membered heterocycle, 3-20 membered heteroaryl and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

In one embodiment the linker L is a branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl or 3-20 membered carbocycle and wherein each chain, 3-20 membered heterocycle, 3-20 membered heteroaryl and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

In one embodiment the linker L is a branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl or 3-20 membered carbocycle and wherein each chain, 3-20 membered heterocycle, 3-20 membered heteroaryl and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

In one embodiment the linker L is a branched or unbranched, saturated or unsaturated, hydrocarbon chain, having from about 1 to about 50 carbon atoms, wherein one or more of the carbon atoms is optionally replaced independently by —O—, —S, —N(R^{a})—, 3-20 membered heterocycle, 3-20 membered heteroaryl or 3-20 membered carbocycle and wherein each chain, 3-20 membered heterocycle, 3-20 membered heteroaryl and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

In one embodiment the linker L is a branched or unbranched, saturated or unsaturated, hydrocarbon chain, having from about 1 to about 50 carbon atoms, wherein one or more of the carbon atoms is optionally replaced independently by —O—, —S, —N(R^{a})—, or R^{b}, wherein each chain and R^{b} is optionally and independently substituted with one or more (e.g. 1, 2, 3, 4, 5 or more) substituents independently selected from the group consisting of azido, halo, and oxo (═O), and carboxy, wherein each R^{b} is independently H or (C₁-C₆)alkyl; and each R^{b} is independently selected from the group consisting of:

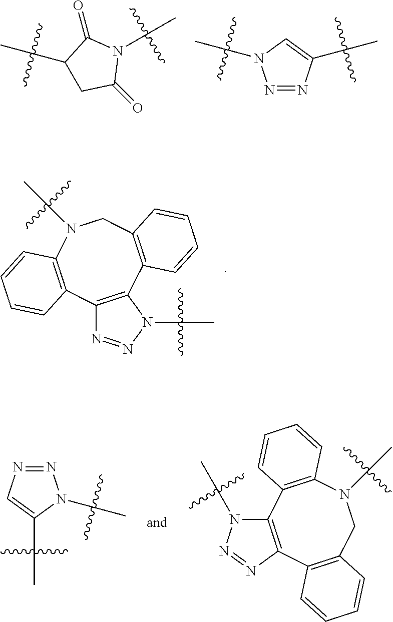 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

In one embodiment the linker L comprises: —C(═O)(CH₂)ₐN(H)C(═O)—; wherein a is 3, 4, 5, 6, 7, or 8.

In one embodiment the linker L comprises —(CH₂CH₂O)_{b}CH₂CH₂C(═O)— or —(CH₂CH₂O)_{b}CH₂CH₂N(H)C(═O)—, wherein b is 2-50.

In one embodiment the linker L comprises phen-1,3-diyl or phen1-1,3,5-triyl.

In one embodiment the linker L comprises:

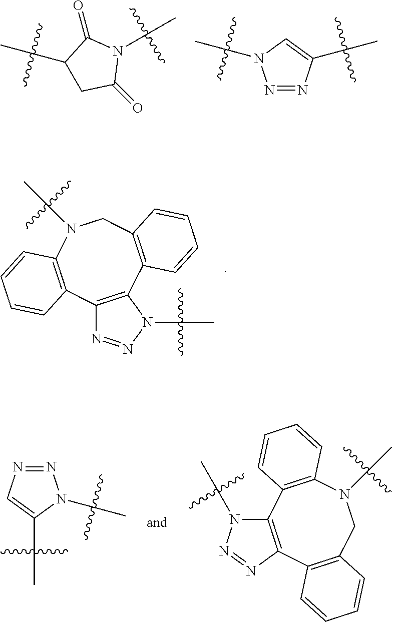 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

In one embodiment each R¹ is linked to L through a carbonyl group of L.

In one embodiment each R² is linked to L through a carbon-carbon bond.

In one embodiment the linker L is selected from the group consisting of:

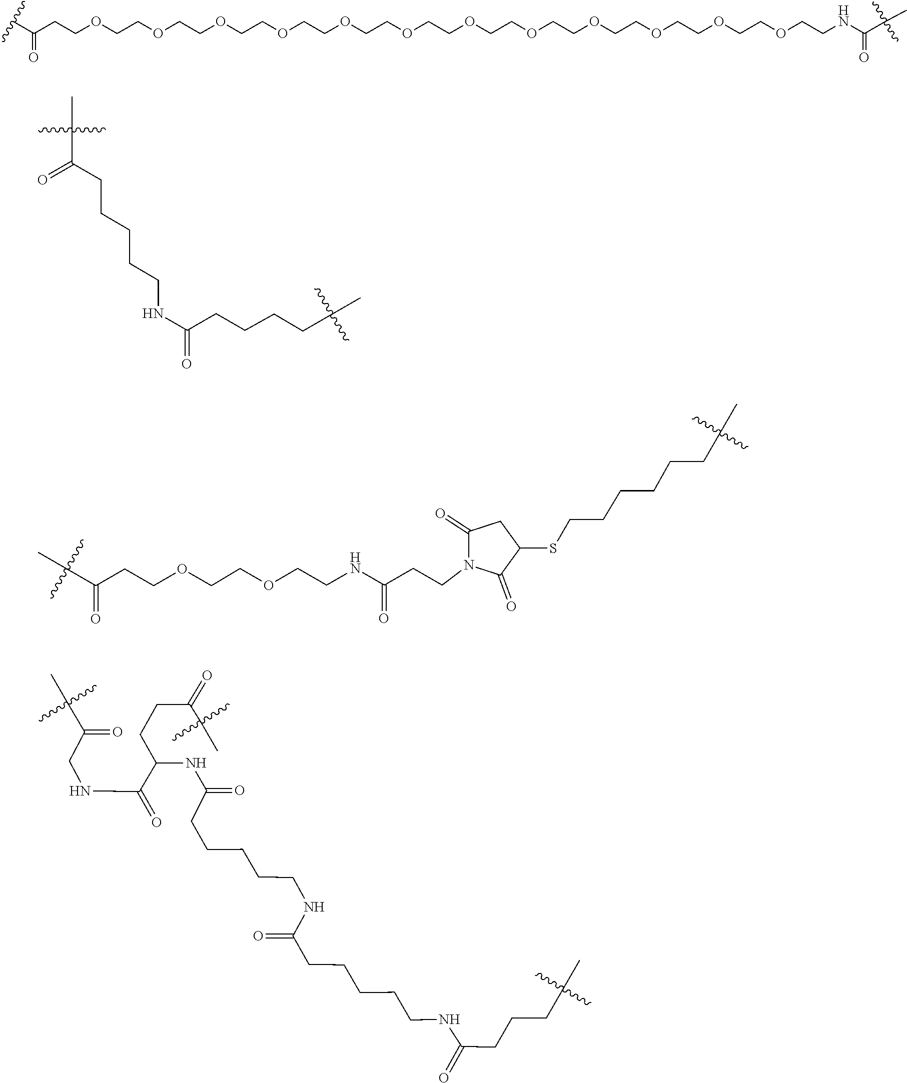 [SMILES: *C(=O)CCOCCOCCOCCOCCOCCOCCOCCOCCOCCOCCOCCOCCNC(*)=O ; *CCCCC(=O)NCCCCCC(*)=O ; *CCCCCCSC1CC(=O)N(CCC(=O)NCCOCCOCCC(*)=O)C1=O ; *CCCC(=O)NCCCCCC(=O)NCCCCCC(=O)NC(CCC(*)=O)C(=O)NCC(*)=O]

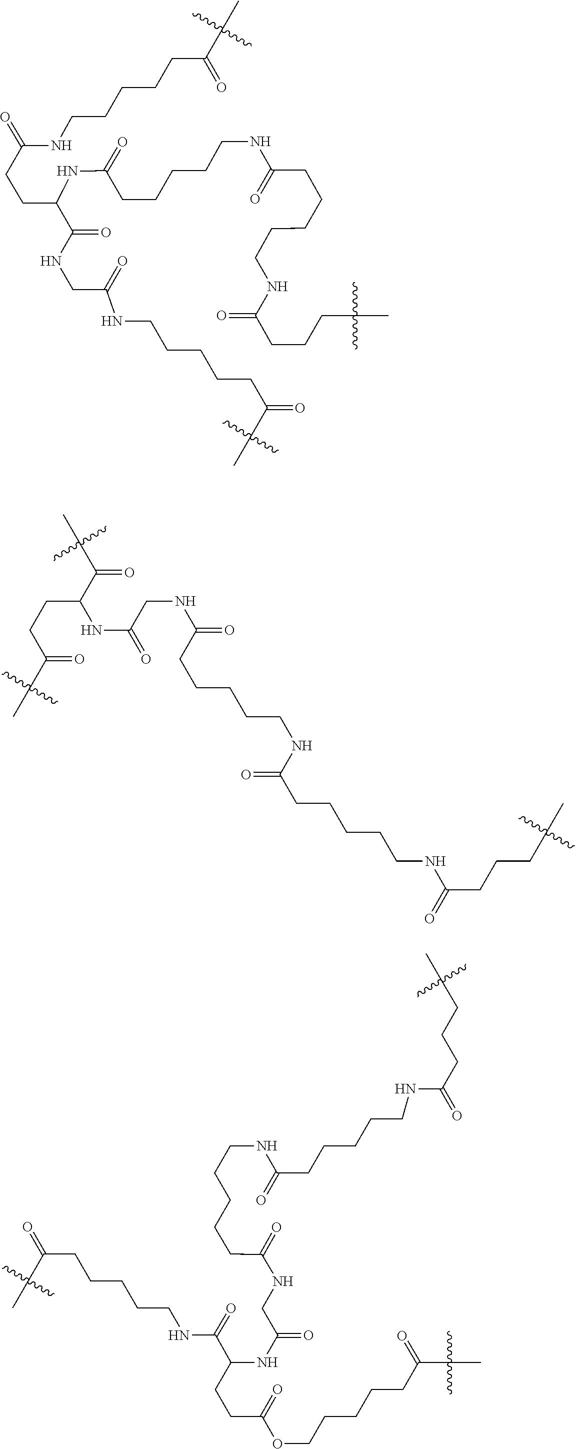 [SMILES: *CCCC(=O)NCCCCCC(=O)NCCCCCC(=O)NC(CCC(=O)NCCCCCC(*)=O)C(=O)NCC(=O)NCCCCCC(*)=O ; *CCCC(=O)NCCCCCC(=O)NCCCCCC(=O)NCC(=O)NC(CCC(*)=O)C(*)=O ; *CCCC(=O)NCCCCCC(=O)NCCCCCC(=O)NCC(=O)NC(CCC(=O)OCCCCCC(*)=O)C(=O)NCCCCCC(*)=O]

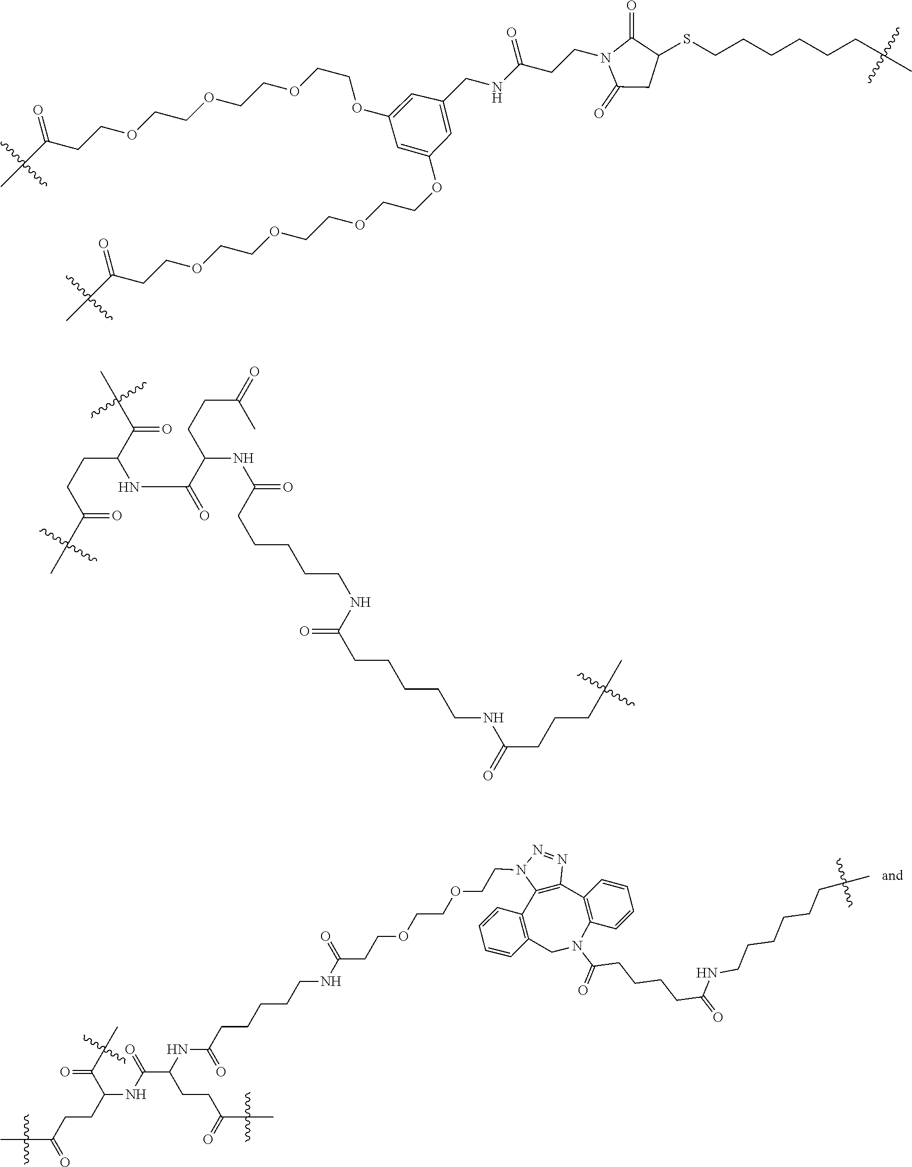 [SMILES: *CCCCCCSC1CC(=O)N(CCC(=O)NCc2cc(OCCOCCOCCOCCC(*)=O)cc(OCCOCCOCCOCCC(*)=O)c2)C1=O ; *CCCC(=O)NCCCCCC(=O)NCCCCCC(=O)NC(CCC(C)=O)C(=O)NC(CCC(*)=O)C(*)=O ; *CCCCCCNC(=O)CCCCC(=O)N1Cc2ccccc2-c2c(nnn2CCOCCOCCC(=O)NCCCCCC(=O)NC(CCC(*)=O)C(=O)NC(CCC(*)=O)C(*)=O)-c2ccccc21]

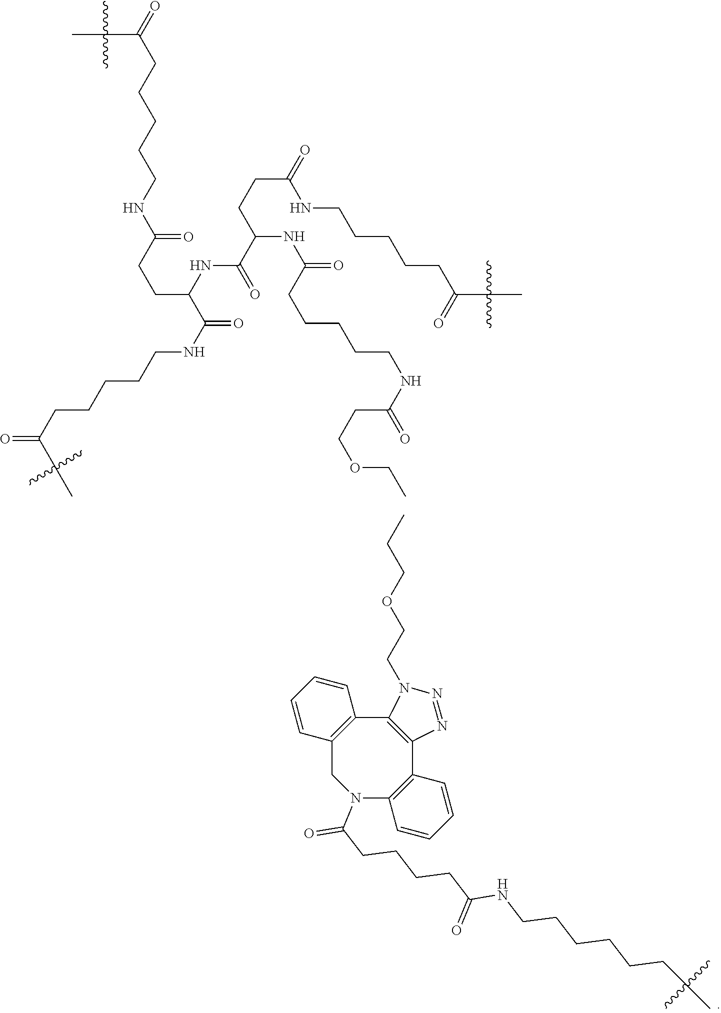

In one embodiment, the linker is comprised of a moiety of formula L₂-Y′Z′-L₁, wherein each of L₂, Y′Z′, and L₁ are as defined herein. Optionally, Y and Z are selected such that Y′Z′ together comprise an oxime functionality. Alternatively, Y and Z are selected such that Y′Z′ together comprise a triazole ring.

### Embodiments of the Invention

One aspect of the invention is a compound of formula I, as set forth about in the Summary of the Invention, or a salt thereof.

In a first embodiment (Embodiment 1; abbreviated as “E1”) the invention provides a compound of formula (I)

(R¹)ₓ-L-(R²)_{y} (I)

or a salt thereof, wherein:
x is 2, 3, 4 or 5;
each R¹ is independently a targeting ligand, which is selected from:
a) a cyclic polypeptide depicted by SEQ ID NO: 1:C*SRNLIDC* (SEQ ID NO: 1), wherein * denotes a disulfide bond linking the two C residues to thereby form the cyclic polypeptide, and
b) any cyclic polypeptide having at least 80% sequence identity (e.g., at least 85%; e.g., at least 87.5%; e.g., at least 90%; e.g., at least 95%; e.g., at least 99% sequence identity) with the polypeptide depicted by SEQ ID NO: 1, under the proviso that both C* residues are present, and wherein * denotes a disulfide bond linking the two C residues to thereby form the cyclic polypeptide; L is a linking group;
y is 1, 2, 3, 4 or 5;
and each R² is independently an oligonucleotide, a label (e.g., a label derivable from fluorescein isothiocyanate (FITC) or Cy5), a phenyl group that is substituted with a formyl (—CHO) group, or a group of formula:

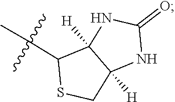 [SMILES: *C1SC[C@@H]2NC(=O)N[C@H]12]

In one embodiment, R₁ is a polypeptide, preferably a cyclic polypeptide, having 6 to 10 amino acid residues, such as 7 to 9 amino acid residues, and preferably about 8 amino acid residues.

In one embodiment, the polypeptide defined in b) is identical to the polypeptide defined in a), except that exactly one of the eight amino acid residues is being exchanged for another amino acid, and thus nonidentical. In that case, the sequence identity of the polypeptide defined in b) is determined as 7 (=identical amino acids)/8 (total positions)*100=87.5%. Thus, in a preferred embodiment, the polypeptide of b), as described herein, has 87.5% sequence identity with the polypeptide defined in a). In one embodiment, the polypeptide has 7 to 10 amino acid residues, and preferably about 8 or 9 amino acid residues, most preferably 8 amino acid residues.

Alternatively, in one embodiment, R₁ can be described as a polypeptide having 7 to 10 amino acid residues, and preferably about 8 or 9 amino acid residues, most preferably 8 amino acid residues, wherein the polypeptide has an amino acid sequence as defined by SEQ ID NO: 1, except that one or more amino acid residues are inserted, deleted or exchanged. Preferably, one to three amino acid residues are inserted, deleted or exchanged, more preferably two or less amino acid residues are inserted, deleted or exchanged, and most preferably one amino acid residue is inserted, deleted or exchanged. Said polypeptide has at least two functional groups capable of forming a bond with each other, such as thiol groups of e.g. cysteine residues, which are capable of forming a disulfide bond, thereby forming a cyclic polypeptide. The functional groups capable of forming a bond with each other are preferably located at or near the N-terminus and C-terminus, respectively.

In one embodiment, R₁ is a polypeptide having a core as defined in a) or b) above, and in addition to that an extension of the polypeptide sequence at either the N-terminus and/or the C-terminus of the polypeptide defined above, such that R₁ overall is a longer polypeptide which comprises R₁ but which is not limited to R₁.

In the cyclic polypeptide as described above, typically, cyclization results from the formation of disulfide bridges between cysteine residues of the peptide such as between the amino acids at positions 1 and 8 of the amino acid sequences described herein, particularly in a) and b) above. Alternatively, an intramolecular bond may be formed in a peptide described herein by a cyclization which is different from a Cys-Cys cyclization. Thus, in an alternative embodiment, the present invention also contemplates peptides, wherein one or both of the Cysteine moieties in the peptide described herein are replaced by a moiety other than Cys. In that embodiment, one or both of the Cys residues at the first and last position (such as positions 1 and 8) of the amino acid sequences described herein may be replaced by (an) amino acid(s) which is (are) able to form an intramolecular bond in the peptide. Preferably, said bond is formed between the amino acids at the first and last position (such as positions 1 and 8) of the amino acid sequences described herein.

Further embodiments (E2-E68) are described below.

E2. The compound or salt of E1, wherein x is 2 or 3. Such compound, e.g. when R2 is a oligonucleotide, such as an siRNA, is particularly advantageous (for a non-limiting and illustrative embodiment, see e.g. Example 12)

E3 The compound or salt of E1, wherein x is 3.

E4. The compound or salt of any one of E1-E3, wherein each R¹ is covalently bound to L (i) through the N terminus of the polypeptide or (ii) through the C terminus of the polypeptide or (iii) through the side chain of an amino acid of the polypeptide.

E5. The compound or salt of any one of E1-E3, wherein each R¹ is covalently bound to L through the N terminus of the polypeptide.

E6. The compound or salt of any one of E1-E3, wherein each R¹ is independently a targeting ligand of formula:

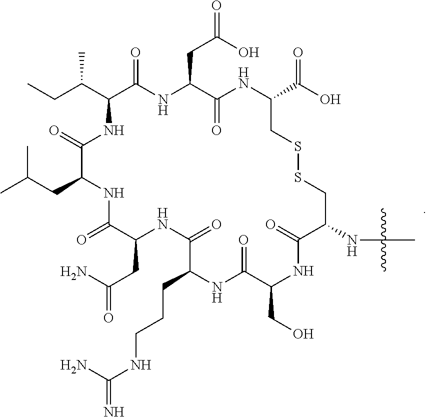 [SMILES: *N[C@H]1CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC1=O]

E7. The compound or salt of any one of E1-E6, wherein each R² is independently an oligonucleotide.

E8. The compound or salt of any one of E1-E6, wherein each R² is independently an siRNA. Such compound is particularly advantageous (for a non-limiting and illustrative embodiment, see e.g. Example 12)

E9. The compound or salt of any one of E7-E8, wherein each oligonucleotide or each siRNA is attached to L through a phosphate P(═O)(OH)₂—O— of each oligonucleotide or each siRNA.

E10. The compound or salt of any one of E1-E6, wherein each R² is independently a group of formula:

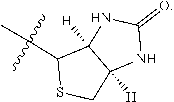 [SMILES: *C1SC[C@@H]2NC(=O)N[C@H]12]

E11. The compound or salt of any one of E1-E6, wherein each R² is independently 3-formylphenyl or 4-formylphenyl.

E12. The compound or salt of any one of E1-E11, wherein L is a branched or unbranched, saturated or unsaturated, hydrocarbon chain, having from about 1 to about 500 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

E13. The compound or salt of any one of E1-E11, wherein L is a branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

E14. The compound or salt of any one of E1-E11, wherein L is a branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

E15. The compound or salt of any one of E1-E11, wherein L is a branched or unbranched, saturated or unsaturated, hydrocarbon chain, having from about 1 to about 50 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

E16. The compound or salt of any one of E1-E11, wherein L is a branched or unbranched, saturated or unsaturated, hydrocarbon chain, having from about 1 to about 50 carbon atoms, wherein one or more of the carbon atoms is optionally replaced independently by —O—, —S, —N(R^{a})—, or R^{b}, wherein each chain and R^{b} is optionally and independently substituted with one or more (e.g. 1, 2, 3, 4, 5 or more) substituents independently selected from the group consisting of azido, halo, ═N(OH), —O(NH₂) and oxo (═O), and carboxy, wherein each R^{b} is independently H or (C₁-C₆)alkyl; and each R^{b} is independently selected from the group consisting of:

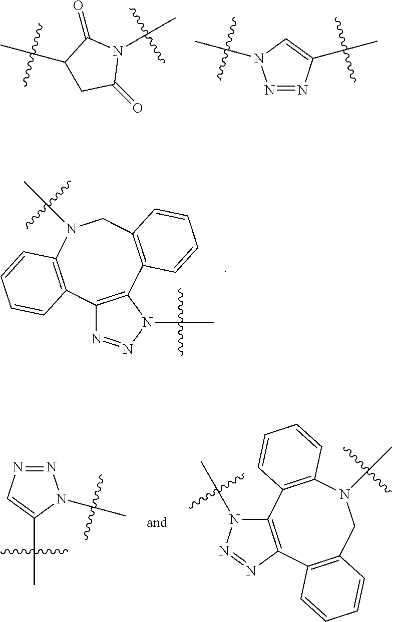 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

E17. The compound or salt of any one of E1-E11, wherein L comprises: —C(═O)(CH₂)ₐN(H) C(═O)—; wherein a is 3, 4, 5, 6, 7, or 8.

E18. The compound or salt of any one of E1-E11, wherein L comprises —(CH₂CH₂O)_{b}CH₂CH₂C(═O)— or —(CH₂CH₂O)_{b}CH₂CH₂N(H)C(═O)—, wherein b is 2-50.

E19. The compound or salt of any one of E1-E11, wherein L comprises phen-1,3-diyl or phen1-1,3,5-triyl.

E20. The compound or salt of any one of E1-E11, wherein L comprises:

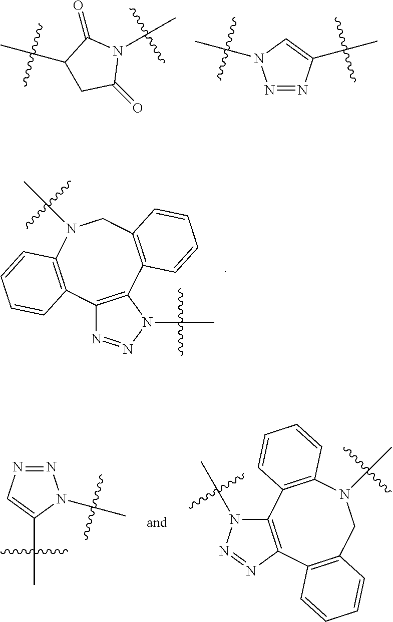 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

E21. The compound or salt of any one of E1-E20, wherein each R¹ is linked to L through a carbonyl group of L.

E22. The compound or salt of any one of E1-E20, wherein each R² is linked to L through a carbon-carbon bond.

E23. The compound or salt of any one of E1-E11, wherein L is selected from the group consisting of:

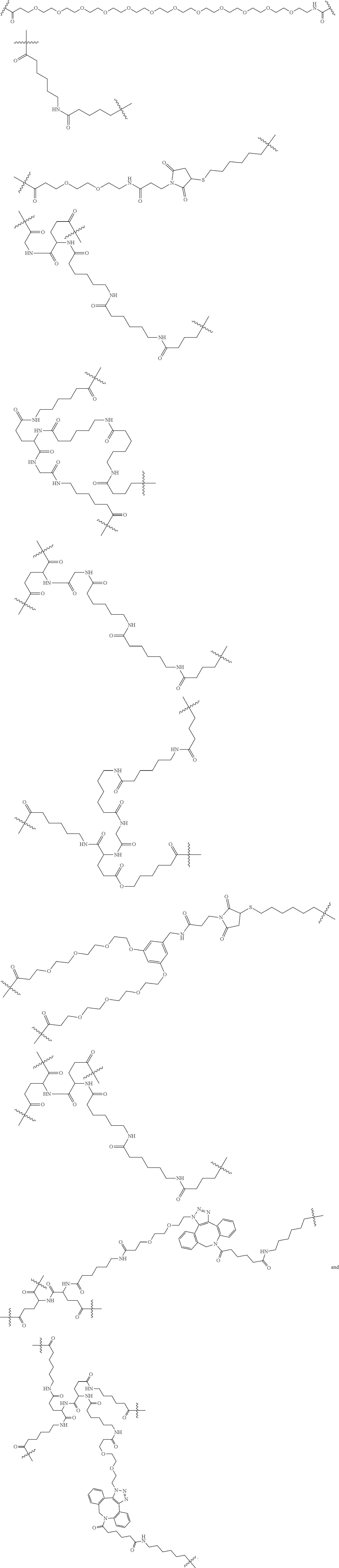

E24. A compound essentially as defined in E1, wherein x is 1, 2 or 3, wherein the compound is selected from the group consisting of:

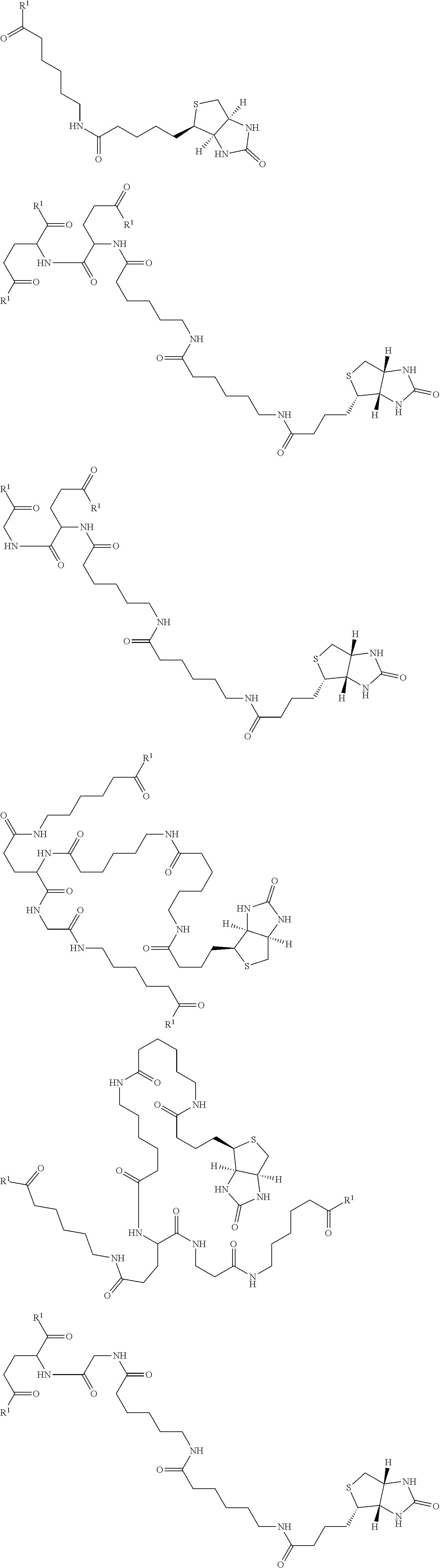

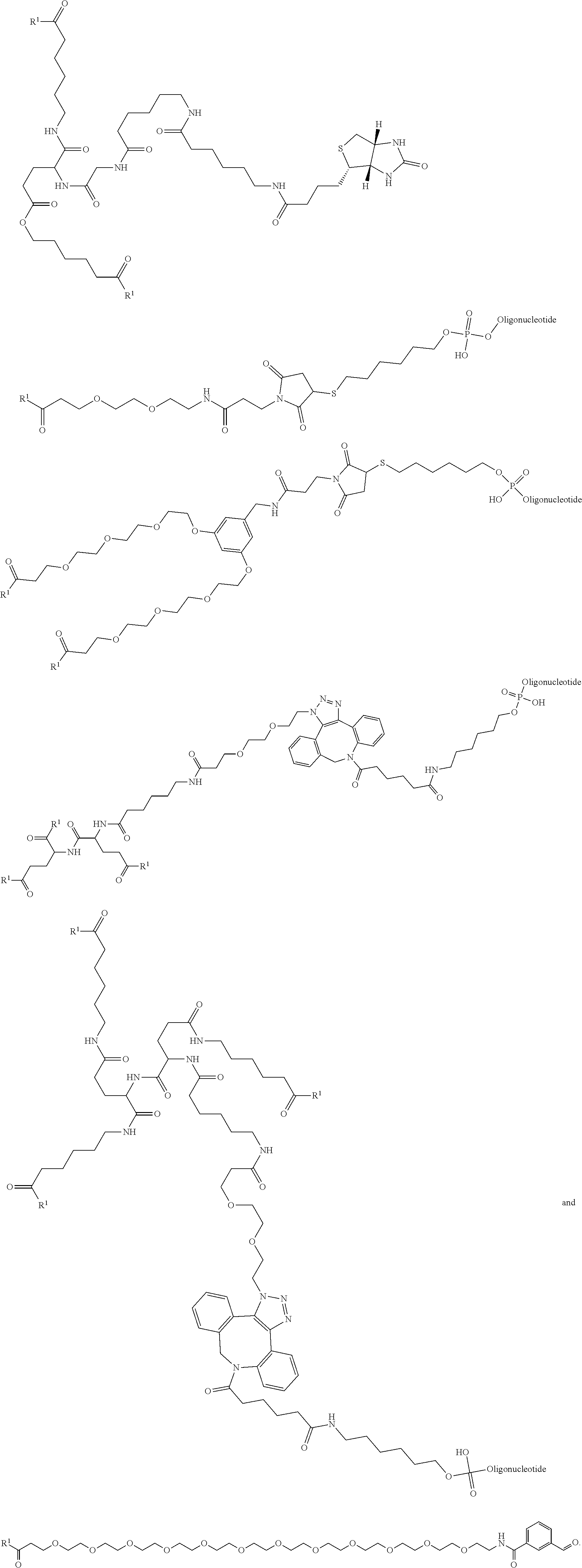

For clarity, in embodiment E24 compounds wherein x=1 are also contemplated. All definitions and moieties are as defined in E1, with the exception of x, which may be in embodiment E24 be selected to be 1. These embodiments, i.e. wherein x is optionally 1 are particularly applicable for the case that R² is independently a label (e.g., a label derivable from fluorescein isothiocyanate (FITC) or Cy5), a phenyl group that is substituted with a formyl (—CHO) group, or a group of formula.

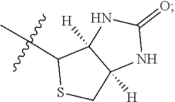 [SMILES: *C1SC[C@@H]2NC(=O)N[C@H]12]

In other words, embodiments, i.e. wherein x is optionally 1 apply with particular preference in case R² is not an oligonucleotide. In preferred embodiments of E24, R² is not an oligonucleotide. In preferred embodiments of E24, is selected to be 1.

In a twenty-fifth embodiment (Embodiment 25; abbreviated as “E25”) the invention provides a process for preparation of a diblock polymer of the following formula (X):

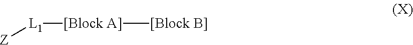

comprising:
a) contacting a compound of structure Va, Vb, Vc, or Vd

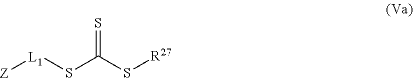

wherein R²⁷ is (C₁-C₁₂)alkyl,

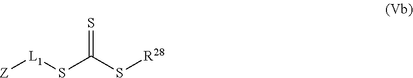

wherein R²⁸ is (C₁-C₁₂)alkyl,

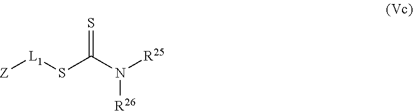

wherein R²⁵ and R²⁶ are independently H, (C₁-C₁₂)alkyl, aryl, or heteroaryl,

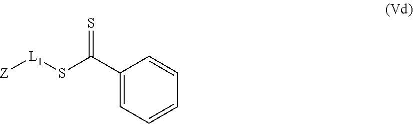

with one or more A monomers selected from the group consisting of:
   i) a polyethyleneglycol methacrylate with 2-20 ethylene glycol units (PEGMA); and
      ii) M², wherein M² is a methacrylate, which is optionally selected among
      a (C₄-C₁₈)alkyl-methacrylate;
      a (C₄-C₁₈)branched alkyl-methacrylate;
      a cholesteryl methacrylate;
      a (C₄-C₁₈)alkyl-methacrylate substituted with one or more fluorine atoms; and
      a (C₄-C₁₈)branched alkyl-methacrylate substituted with one or more fluorine atoms;
in the presence of a free radical to provide a first product;
b) contacting the first product with one or more B monomers of formulae B1, B2 and B3

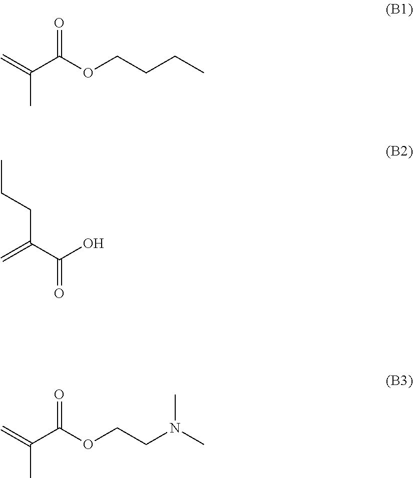

in the presence of a free radical to provide a second product, and
c) optionally contacting the second product with a free radical source (e.g., AIBN) to remove the chain transfer agent and provide the diblock polymer of formula (X);
wherein:
   Block A comprises one or more residues of A monomers and has a molecular weight of from about 1 kDa to about 25 kDa;
   Block B comprises one or more residues of monomers B1, B2, and B3 and has a molecular weight of from about 1 kDa to about 25 kDa.
   L₁ is a linking moiety;
   Z is a functional group that is optionally protected with a protecting group and is capable of reacting with Y of a compound of formula (XII):

T-L₂-Y (XII)
to form a conjugate wherein:
   T is a ligand (optionally targeting ligand);
   Y is a functional group that is capable of reacting with Z to form a conjugate; and
   L₂ is absent or is a linking moiety.

A “free radical source” as used herein is not particularly limited. Suitable free radical sources include without limitation Azobisisobutyronitrile (AIBN), 1,1-Azobis(cyanocyclohexane) (ACHN/VAZO-88), 4,4′-azobis(4-cyanovaleric acid) (ACVA), Potassium persulfate (K₂S₂O₈).

The term “chain transfer agent” (CTA), as used herein, refers to a molecular entity suitable to afford control over the generated molecular weight and polydispersity during a free-radical polymerization. Typical CTAs suitable in the present invention are, in non-limiting examples, thiocarbonylthio compounds (or similar. A CTA is also sometimes referred to as “RAFT agent” in the art. Compounds Va, Vb, Vc and Vd, as introduced above, are non-liming examples of CTA suitable in the process according to the present invention.

While step c) is optional, it is preferred that the process of the present invention actually comprises step c) as well. The result of a process comprising step c) is the compound of formula (I), which is devoid of a CTA moiety. In the alternative embodiment that step c) is not present, the resulting polymer still comprises a moiety derived from the CTA, while otherwise identical to the compound of formula (I).

The product of this process, whether or not comprising step c), is useful for coupling with a T moiety, as described herein below. Typically, the T moiety is conjugated to the polymer obtained by the above process in a step of post-polymerization conjugation:

This process and all its embodiments is particularly helpful for the preparation of a polymer with a T moiety that is not susceptible to RAFT polymerization as described e.g. in WO 2015/017519 A1. This is the case for example when T is selected to be an oligonucleotide or a peptide, such as the cyclic peptide as described herein. In particular, the process according to the present invention is characterized in that T is conjugated to the polymer after the polymerization reaction (also referred to as RAFT polymerization). Thus, the process according to the present invention can also be referred to as post-polymerization conjugation. The inventors made endosome release polymer (ERP) characterized by such T moieties available through the present invention. Thus, the present invention provides a process for making endosome release polymer (ERP), wherein the ERP obtainable by such process is characterized in comprising a ligand which is optionally an oligonucleotide or a peptide. Even though oligonucleotides and peptides, among other ligands, are not susceptible to RAFT polymerization, the present invention makes ERP with such ligands available. The process of the present invention is also an economic process for making ERP comprising a relatively expensive or valuable ligand, optionally selected among an oligonucleotide and a peptide. Conjugation of such expensive or valuable ligand at the final step of synthesis as opposed to the beginning of RAFT polymerization process significantly reduces the overall production cost because the yield of RAFT polymerization does not affect the amount of ligand required to make the final product.

While the second product, i.e. the product resulting from step b) has a moiety of a radical starter, originating from a compound of structure Va, Vb, Vc, or Vd attached to it, the contacting of the second product with one or more free radicals provides the diblock polymer of formula (X). Thus addition of the free radical removes this group and changes the terminal group of the polymer to a proton. Preferably, the contacting of the second product with one or more free radicals occurs under protic conditions. This is advantageous for changing polymer end group, to substitute the RAFT agent by a proton.

The process of making the diblock polymer of formula (X), as described in E25 and any of its embodiments as described herein can be referred to as “Reversible Addition-Fragmentation chain Transfer” (or RAFT) polymerization. According to the present invention, RAFT is used in synthesizing ethylenic backbone polymers of this invention. RAFT is a living polymerization process. RAFT comprises a free radical degenerative chain transfer process. In some embodiments, RAFT procedures for preparing a polymer described herein employs thiocarbonylthio compounds such as, without limitation, dithioesters, dithiocarbamates, trithiocarbonates and xanthates to mediate polymerization by a reversible chain transfer mechanism. In certain instances, reaction of a polymeric radical with the C═S group of any of the preceding compounds leads to the formation of stabilized radical intermediates. Typically, these stabilized radical intermediates do not undergo the termination reactions typical of standard radical polymerization but, rather, reintroduce a radical capable of re-initiation or propagation with monomer, reforming the C═S bond in the process. In most instances, this cycle of addition to the C═S bond followed by fragmentation of the ensuing radical continues until all monomer has been consumed or the reaction is quenched. Generally, the low concentration of active radicals at any particular time limits normal termination reactions.

In some embodiments, polymers of the present invention have a low polydispersity index (PDI or DI) or differences in chain length. Polydispersity index can be determined in any suitable manner, e.g., by dividing the weight average molecular weight of the polymer chains by their number average molecular weight. The number average molecule weight is sum of individual chain molecular weights divided by the number of chains. The weight average molecular weight is proportional to the square of the molecular weight divided by the number of molecules of that molecular weight. Since the weight average molecular weight is always greater than the number average molecular weight, polydispersity is always greater than or equal to one. As the numbers come closer and closer to being the same, i.e., as the polydispersity approaches a value of one, the polymer becomes closer to being monodisperse in which every chain has exactly the same number of constitutional units. Polydispersity values approaching one are achievable using radical living polymerization. Methods of determining polydispersity, such as, but not limited to, size exclusion chromatography, dynamic light scattering, matrix-assisted laser desorption/ionization chromatography and electrospray mass chromatography are well known in the art. In some embodiments, the polymers (e.g., membrane destabilizing polymers) provided herein have a polydispersity index (PDI) of less than 2.0, or less than 1.8, or less than 1.6, or less than 1.5, or less than 1.4, or less than 1.3, or less than 1.2. In some embodiments, the polymer is a block copolymer (e.g., endosome release copolymer) comprising a hydrophilic block and a hydrophobic block and having a polydispersity index (PDI) of less than 2.0, or less than 1.8, or less than 1.6, or less than 1.5, or less than 1.4, or less than 1.3, or less than 1.2. Polymerization processes described herein optionally occur in any suitable solvent or mixture thereof. Suitable solvents include water, alcohol (e.g., methanol, ethanol, n-propanol, isopropanol, butanol), tetrahydrofuran (THF) dimethyl]sulfoxide (DMSO), dimethylformamide (DMF), acetone, acetonitrile, hexamethylphosphoramide, acetic acid, formic acid, hexane, cyclohexane, benzene, toluene, dioxane, methylene chloride, ether (e.g., diethyl ether), chloroform, and ethyl acetate. In one aspect, the solvent includes water, and mixtures of water and water-miscible organic solvents such as DMF.

E26. The process of E25, wherein PEGMA is

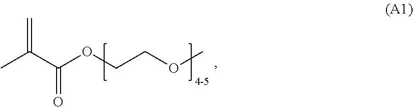

and/or M² is

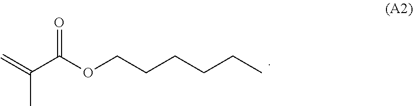

In other words, in this embodiment, the A monomers are selected from the group consisting of monomers of formulae A1 and A2.

In one embodiment, the process of E26 yields a compound of formula (XI):

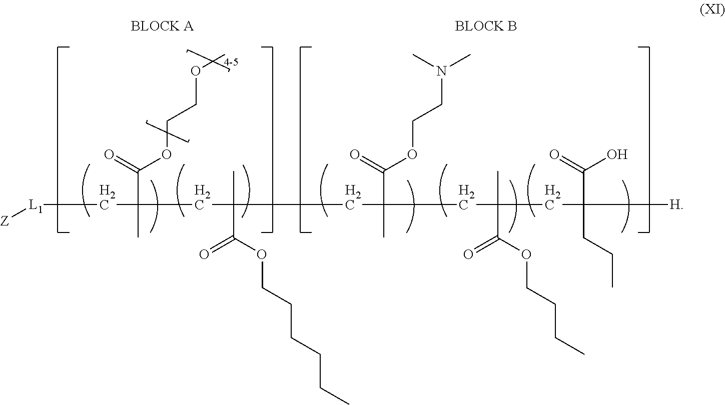

Rounded brackets denote the monomer moieties. Squared brackets denote Blocks A and B, respectively, of the diblock polymer. Stoichiometry (m and n; q, r and s) and molecular weights (v and w) are as defined in E25.

E27. The process of E25 or E26 wherein Z is a functional group that is protected with a protecting group, wherein the process further comprises removing the protecting group from Z.

E28. The process of E25 or E26 wherein Z is a functional group that is not protected with a protecting group.

E29. The process of E27 or E28 further comprising reacting the diblock polymer of formula (XI) with a compound of formula (XII):

T-L₂-Y (XII)

under conditions where Z reacts with Y to form a group Y′Z′ and yield a conjugate of formula:

T-L₂-Y′Z′-L₁-[Block A]-[Block B].

In one embodiment, this process yields the endosome release polymer described in E49 and any of its embodiments.

In one embodiment, the conjugate is a conjugate of formula (XIII):

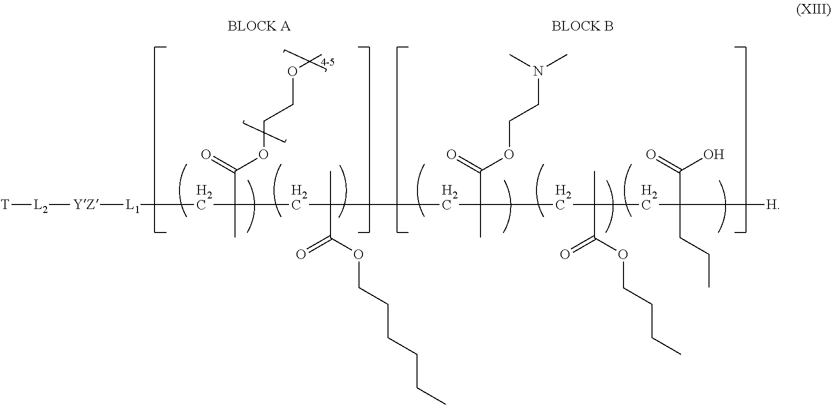

Rounded brackets denote the monomer moieties. Squared brackets denote Blocks A and B, respectively, of the diblock polymer.

E30. The process of E29, wherein Block A has a molecular weight of from about 1 kDa to about 25 kDa; and Block B has a molecular weight of from about 1 kDa to about 25 kDa.

E31. The process of E29, wherein Block A has formula:

-[PEGMAₘ-M²ₙ]ᵥ-

wherein:
PEGMA is polyethyleneglycol methacrylate residue with 2-20 ethylene glycol units; M² is a methacrylate residue selected from the group consisting of
   a (C₄-C₁₈)alkyl-methacrylate residue;
   a (C₄-C₁₈)branched alkyl-methacrylate residue;
   a cholesteryl methacrylate residue;
   a (C₄-C₁₈)alkyl-methacrylate residue substituted with one or more fluorine atoms; and
   a (C₄-C₁₈)branched alkyl-methacrylate residue substituted with one or more fluorine atoms; and
   v is 1 to 25 kDa;
and wherein Block B has formula:

-[DMAEMA_{q}-PAAᵣ-BMAₛ]_{w}
wherein:
   BMA is butyl methacrylate residue;
   PAA is propyl acrylic acid residue;
   DMAEMA is dimethylaminoethyl methacrylate residue;
   m and n are each a mole fraction greater than 0, wherein m is greater than n and m+n=1;
   q is a mole fraction of 0.2 to 0.75;
   r is a mole fraction of 0.05 to 0.6;
   s is a mole fraction of 0.2 to 0.75;
   q+r+s=1; and
   w is 1 to 25 kDa.
   E31 can also be described by the following formula:

T-L₂-Y′Z′-L₁ [symbol] (PEGMA)ₙ-(M²)ₙ [symbol] ᵥ [symbol] (DMAEMA)_{q}-(PAA)ᵣ)-BMA)ₛ [symbol] _{w}

Rounded brackets denote the monomer moieties. Squared brackets denote Blocks A and B, respectively, of the diblock polymer.

E32. The process of any one of E25-E31, wherein Z and Y are selected so that Y′Z′ comprises an oxime functionality.

E33. The process of any one of E25-E31, wherein Z and Y are selected so that Y′Z′ comprises a triazole ring.

E34. The process of any one of E25-E27 and E29-E32, wherein Z comprises an aminooxy group that is protected, and the protecting group is on the nitrogen of the aminooxy group.

E35. The process of any one of E29-E32, wherein Z comprises an aminooxy group, Y comprises a ketone group or an aldehyde group, and Y′Z′ comprises an oxime functional group.

E36. The process of any one of E29-E32, wherein Z comprises a ketone group or an aldehyde group, Y comprises an aminooxy group, and Y′Z′ comprises an oxime functional group.

E37. The process of E29 or E33, wherein Z comprises an alkyne group, Y comprises an azide group, and Y′Z′ comprises a triazole ring.

E38. The process of E29 or E33, wherein Z comprises an azide group, Y comprises an alkyne group, and Y′Z′ comprises a triazole ring.

E39. The process of any one of any one of E29-E38, wherein T is a targeting ligand or a labeling agent.

E40. The process of any one of E29-E38, wherein the targeting ligand is selected from the group of an oligonucleotide, a peptide, a saccharide, and a small molecule.

E41. The process of any one of E29-E38, wherein the targeting ligand comprises a cyclic peptide.

E42. The process of any one of E29-E38, wherein the targeting ligand comprises a cyclic peptide as described in E1.

E43. The process of any one of E29-E38, wherein the targeting ligand comprises a cyclic peptide as described in E6.

E44. The process of any one of E29-E38, wherein the labeling agent is selected from the group consisting of a fluorophore, a chromophore, and a radionucleotide.

E45. The process of any one of E29-E38, wherein the labeling agent is derivable from fluorescein isothiocyanate (FITC) or Cy5.

E46. A product prepared by the process of any one of E25-E45.

E47. A conjugate prepared by the process of any one of E29-E45.

E48. A composition comprising a plurality of conjugates as defined in any one of E29-E45.

E49. The composition of E48, wherein at least one of the conjugates comprises a labelling agent.

In a fiftieth embodiment (Embodiment 50; abbreviated as “E50”) the invention provides a compound of formula (I)

(R¹)ₓ-L-(R²)ᵥ (I)

or a salt thereof, wherein:
x is 1, 2, 3, 4 or 5;
each R¹ is independently a targeting ligand, which is selected from
a) a cyclic polypeptide depicted by SEQ ID NO: 1:C*SRNLIDC* (SEQ ID NO: 1), wherein * denotes a disulfide bond linking the two C residues to thereby form the cyclic polypeptide,
b) any cyclic polypeptide having at least 80% sequence identity (e.g., at least 85%; e.g., at least 87.5%; e.g., at least 90%; e.g., at least 95%; e.g., at least 99% sequence identity) with the polypeptide depicted by SEQ ID NO: 1, under the proviso that both C* residues are present, and wherein * denotes a disulfide bond linking the two C residues to thereby form the cyclic polypeptide;
L is a linking group; and
y is 1, 2, 3, 4 or 5;
and each R² is independently an endosomal release polymer.

Preferably, the compound or salt of E50 is available by the process of E29 and any of its embodiments.

E51. The compound or salt of E50, wherein the endosomal release polymer comprises a diblock polymer comprising Block A and Block B having the following formula:

[Block A]-[Block B]

wherein:
Block A comprises one or more residues of monomers A1 and A2 as described in E25 and has a molecular weight of from about 1 kDa to about 25 kDa; and
Block B comprises one or more residues of monomers B1, B2, and B3 as described in E25 and has a molecular weight of from about 1 kDa to about 25 kDa.

In one embodiment, said compound of Formula (I) is obtainable by a process as described herein.

E52. The compound or salt of E50, wherein the endosomal release polymer has formula XX:

-[PEGMAₘ-M²ₙ]ᵥ-[DMAEMA_{q}-PAAᵣ-BMAₛ]_{w} (XX)

wherein:
PEGMA is polyethyleneglycol methacrylate residue with 2-20 ethylene glycol units; M² is a methacrylate residue selected from the group consisting of
   a (C₄-C₁₈)alkyl-methacrylate residue;
   a (C₄-C₁₈)branched alkyl-methacrylate residue;
   a cholesteryl methacrylate residue;
   a (C₄-C₁₈)alkyl-methacrylate residue substituted with one or more fluorine atoms; and
   a (C₄-C₁₈)branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa; and
w is 1 to 25 kDa

E53. The compound or salt of E51, wherein Block A comprises one or more residues of monomers of formulae A1 and A2 as described in E26.

E54. A pharmaceutical composition comprising a compound as described in any one of E1-E24 and E50-E53 or a pharmaceutically acceptable salt thereof, and a pharmaceutically acceptable carrier.

E55. A pharmaceutical composition comprising a first compound as described in any one of E1-E24 or a pharmaceutically acceptable salt thereof, wherein each R² is independently an oligonucleotide and a second compound as described in any one of E50-E53 or a pharmaceutically acceptable salt thereof.

E56. A method to deliver an oligonucleotide to an animal comprising administering a compound as described in any one of E1-E24 or a pharmaceutically acceptable salt thereof, to the animal.

E57. A method to deliver an oligonucleotide to cells that express platelet-derived growth factor receptor (PDGFR) in an animal comprising administering a compound as described in any one of E1-E24 or a pharmaceutically acceptable salt thereof, to the animal.

E58. The method of E57, wherein the platelet-derived growth factor receptor (PDGFR) is platelet-derived growth factor receptor alpha (PDGFRα).

E59. The method of E57, wherein the platelet-derived growth factor receptor (PDGFR) is platelet-derived growth factor receptor beta (PDGFRβ).

E60. The method of E57, wherein the cells that express PDGFR are hepatic stellate cells (HSC), endothelial cells, fibroblasts or tumor cells.

E61. A method to treat a disease involving PDGFR expressing cells (e.g., liver fibrosis, non-alcoholic steatohepatitis (NASH), clear cell renal cell carcinoma, kidney fibrosis, or alcoholic steatohepatitis (ASH)) comprising administering a compound as described in any one of E1-E24 or a pharmaceutically acceptable salt thereof, to the animal.

E62. A compound as described in any one of E1-E24 or a pharmaceutically acceptable salt thereof for delivering an oligonucleotide to an animal.

E63. A compound as described in any one of E1-E24 or a pharmaceutically acceptable salt thereof for delivering an oligonucleotide to cells that express platelet-derived growth factor receptor (PDGFR).

E64. A compound as described in any one of E1-E24 or a pharmaceutically acceptable salt thereof for the prophylactic or therapeutic treatment of a disease involving PDGFR expressing cells (e.g., liver fibrosis, non-alcoholic steatohepatitis (NASH), clear cell renal cell carcinoma, kidney fibrosis, or alcoholic steatohepatitis (ASH)).

E65. The use of a compound as described in any one of E1-E24 or a pharmaceutically acceptable salt thereof to prepare a medicament for delivering an oligonucleotide to an animal.

E66. The use of a compound as described in any one of E1-E24 or a pharmaceutically acceptable salt thereof to prepare a medicament for delivering an oligonucleotide to cells that express platelet-derived growth factor receptor (PDGFR).

E67. The use of a compound as described in any one of E1-E24 or a pharmaceutically acceptable salt thereof to prepare a medicament for treating a disease involving PDGFR expressing cells (e.g., liver fibrosis, non-alcoholic steatohepatitis (NASH), clear cell renal cell carcinoma, kidney fibrosis, or alcoholic steatohepatitis (ASH)).

E68. The method, compound of use of any one of E56-E67, which comprises the combined use of a first compound as described in any one of E1-E24, wherein each R² is independently an oligonucleotide, with a second compound as described in any one of E50-E53.

In another embodiment (Embodiment 101; abbreviated as “E101”) the invention provides a compound of formula (I)

(R¹)ₓ-L-(R²)_{y} (I)

wherein:
x is 1, 2, 3, 4 or 5;
each R¹ is independently a targeting ligand of formula:

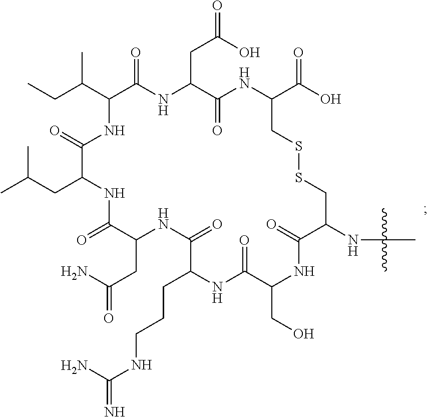 [SMILES: *NC1CSSCC(C(=O)O)NC(=O)C(CC(=O)O)NC(=O)C(C(C)CC)NC(=O)C(CC(C)C)NC(=O)C(CC(N)=O)NC(=O)C(CCCNC(=N)N)NC(=O)C(CO)NC1=O]

L is a linking group; and
y is 1, 2, 3, 4 or 5; and each R² is independently an oligonucleotide, a label (e.g., a label derivable from fluorescein isothiocyanate (FITC) or Cy5), a phenyl group that is substituted with a formyl (—CHO) group, or a group of formula:

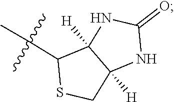 [SMILES: *C1SC[C@@H]2NC(=O)N[C@H]12]

or y is 1, 2, 3, 4, or 5 and R² is an endosomal release agent including polymers and peptides or a salt thereof.

Further embodiments (E102-E122) are described below.

E102. The compound or salt of E101, wherein each R¹ is independently a targeting ligand of formula:

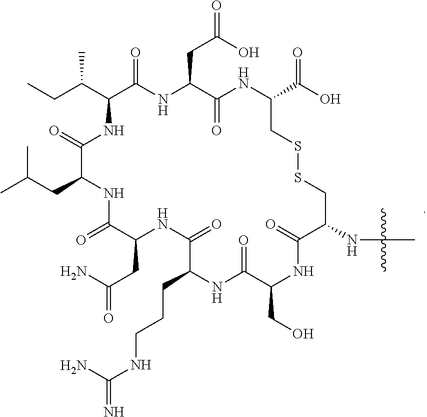 [SMILES: *N[C@H]1CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC1=O]

E103. The compound or salt of E101 or E102, wherein each R² is independently an oligonucleotide.

E104. The compound or salt of E101 or E102, wherein each R² is independently an siRNA.

E105. The compound of E103 or E104, wherein each oligonucleotide or each siRNA is attached to L through a phosphate P(═O)(OH)₂—O— of each oligonucleotide or each siRNA.

E106. The compound or salt of E101 or E102, wherein each R² is independently a group of formula:

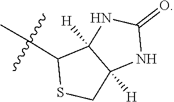 [SMILES: *C1SC[C@@H]2NC(=O)N[C@H]12]

E107. The compound or salt of E101 or E102, wherein each R² is independently 3-formylphenyl or 4-formylphenyl.

E108. The compound or salt of any one of E101-E107, wherein Lisa branched or unbranched, saturated or unsaturated, hydrocarbon chain, having from about 1 to about 500 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

E109. The compound or salt of any one of E101-E107, wherein Lisa branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

E110. The compound or salt of any one of E101-E107, wherein Lisa branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

E111. The compound or salt of any one of E101-E107, wherein Lisa branched or unbranched, saturated or unsaturated, hydrocarbon chain, having from about 1 to about 50 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

E112. The compound or salt of any one of E101-E107, wherein Lisa branched or unbranched, saturated or unsaturated, hydrocarbon chain, having from about 1 to about 50 carbon atoms, wherein one or more of the carbon atoms is optionally replaced independently by —O—, —S, —N(R^{a})—, or R^{b}, wherein each chain and R^{b} is optionally and independently substituted with one or more (e.g. 1, 2, 3, 4, 5 or more) substituents independently selected from the group consisting of azido, halo, ═N(OH), —O(NH2) and oxo (═O), and carboxy, wherein each R^{b} is independently H or (C₁-C₆)alkyl; and each R^{b} is independently selected from the group consisting of:

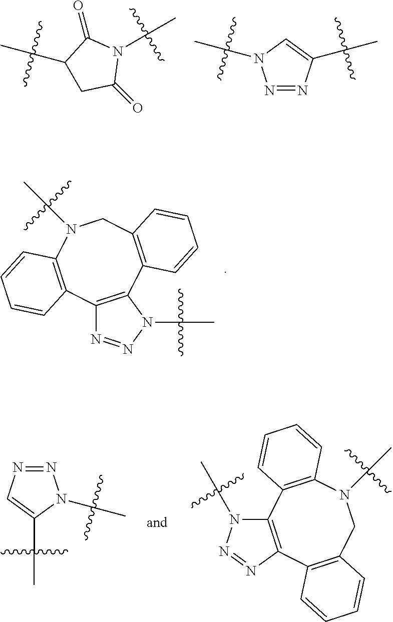 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

E113. The compound or salt of any one of E101-E112, wherein L comprises: —C(═O)(CH₂)ₐN(H) C(═O)—; wherein a is 3, 4, 5, 6, 7, or 8.

E114. The compound or salt of any one of E101-E113, wherein L comprises —(CH₂CH₂O)_{b}CH₂CH₂C(═O)— or —(CH₂CH₂O)_{b}CH₂CH₂N(H)C(═O)—, wherein b is 2-50.

E115. The compound or salt of any one of E101-E114, wherein L comprises phen-1,3-diyl or phen1-1,3,5-triyl.

E116. The compound or salt of any one of E101-E115, wherein L comprises:

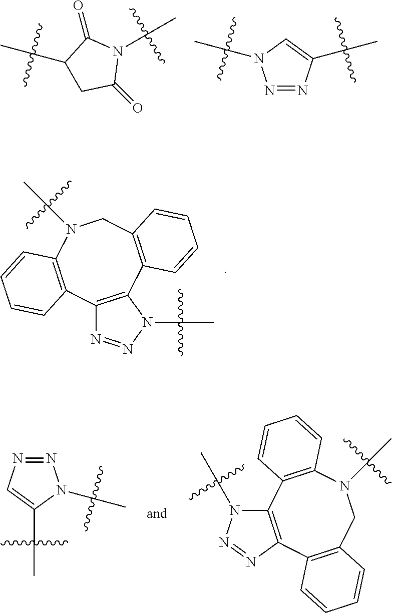 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

E117. The compound or salt of any one of E101-E116, wherein each R¹ is linked to L through a carbonyl group of L.

E118. The compound or salt of any one of E101-E117, wherein each R² is linked to L through a carbon-carbon bond.

E119. The compound or salt of any one of E101-E118, wherein L is selected from the group consisting of:

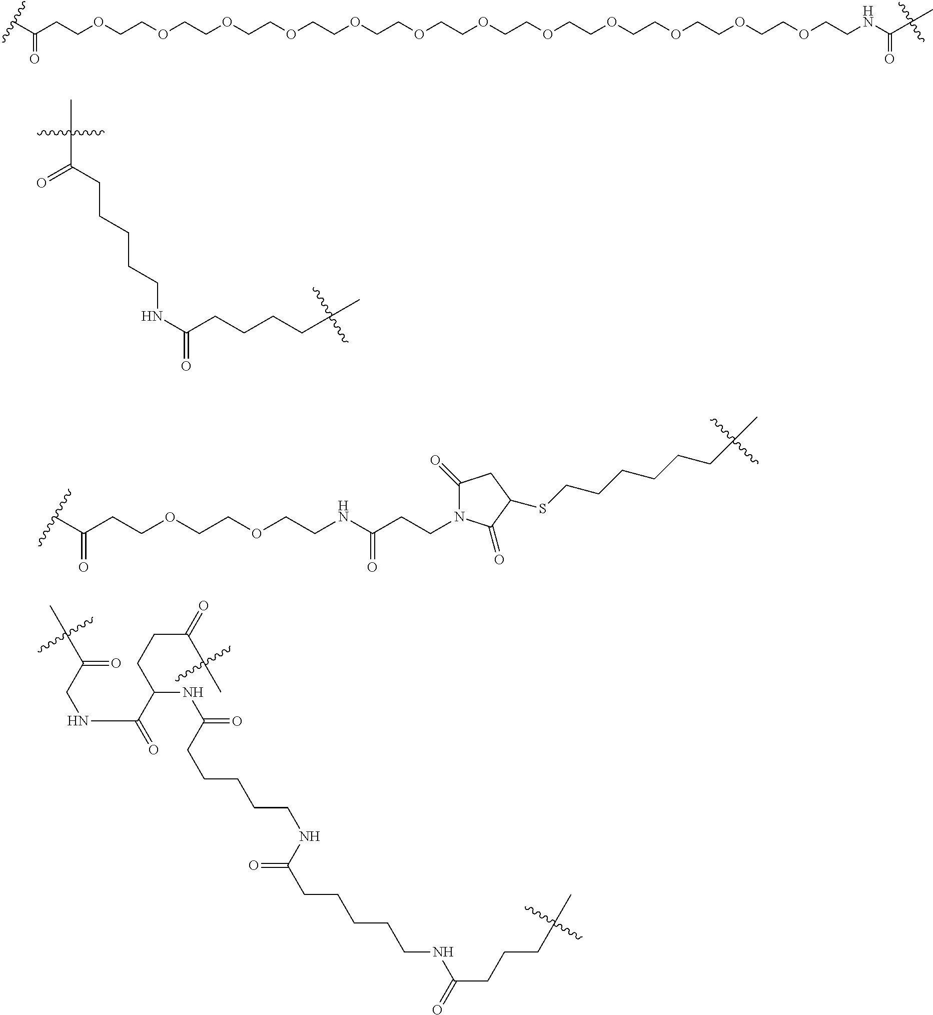 [SMILES: *C(=O)CCOCCOCCOCCOCCOCCOCCOCCOCCOCCOCCOCCOCCNC(*)=O ; *CCCCC(=O)NCCCCCC(*)=O ; *CCCCCCSC1CC(=O)N(CCC(=O)NCCOCCOCCC(*)=O)C1=O ; *CCCC(=O)NCCCCCC(=O)NCCCCCC(=O)NC(CCC(*)=O)C(=O)NCC(*)=O]

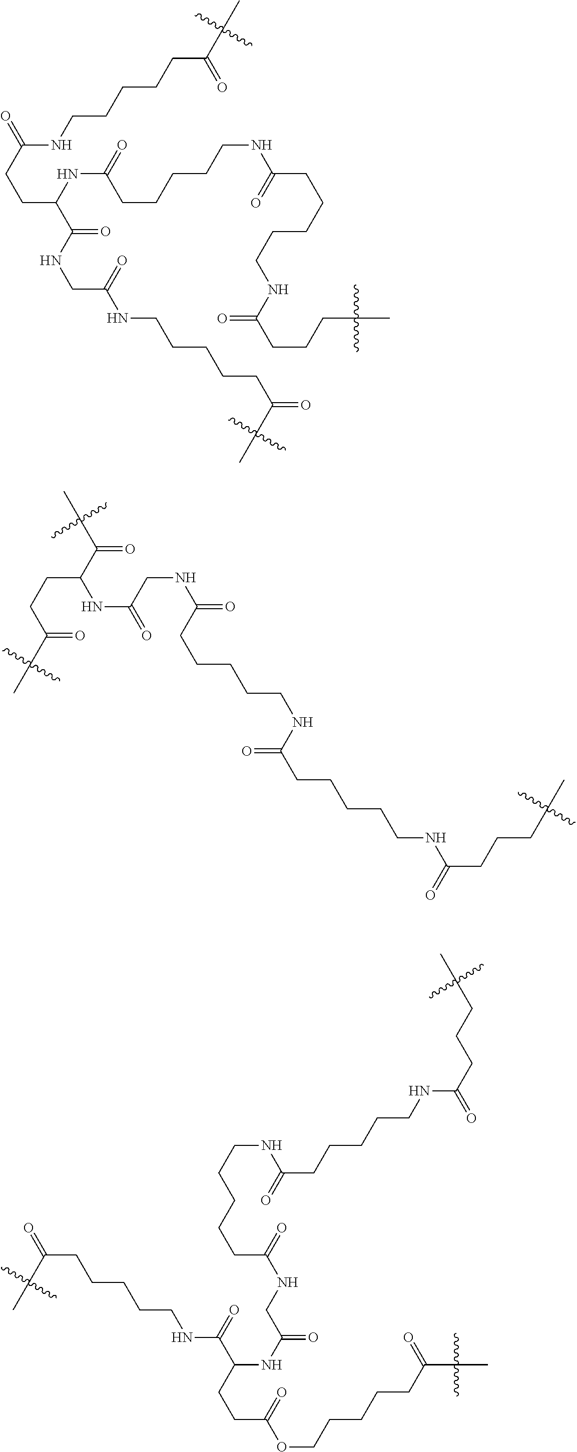 [SMILES: *CCCC(=O)NCCCCCC(=O)NCCCCCC(=O)NC(CCC(=O)NCCCCCC(*)=O)C(=O)NCC(=O)NCCCCCC(*)=O ; *CCCC(=O)NCCCCCC(=O)NCCCCCC(=O)NCC(=O)NC(CCC(*)=O)C(*)=O ; *CCCC(=O)NCCCCCC(=O)NCCCCCC(=O)NCC(=O)NC(CCC(=O)OCCCCCC(*)=O)C(=O)NCCCCCC(*)=O]

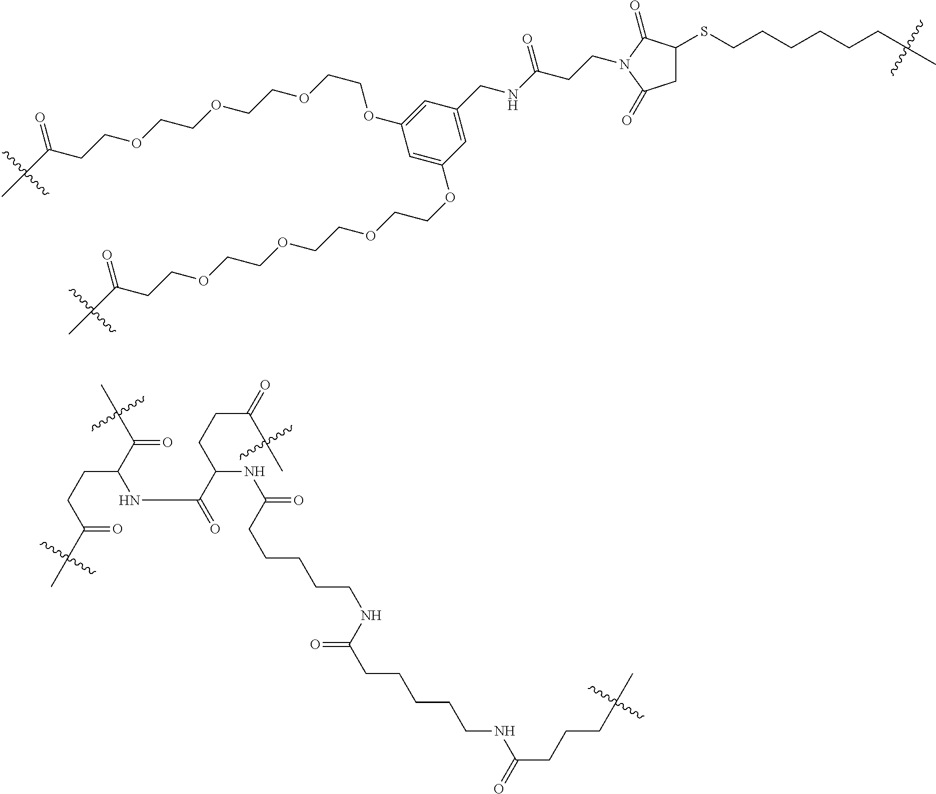 [SMILES: *CCCCCCSC1CC(=O)N(CCC(=O)NCc2cc(OCCOCCOCCOCCC(*)=O)cc(OCCOCCOCCOCCC(*)=O)c2)C1=O ; *CCCC(=O)NCCCCCC(=O)NCCCCCC(=O)NC(CCC(*)=O)C(=O)NC(CCC(*)=O)C(*)=O]

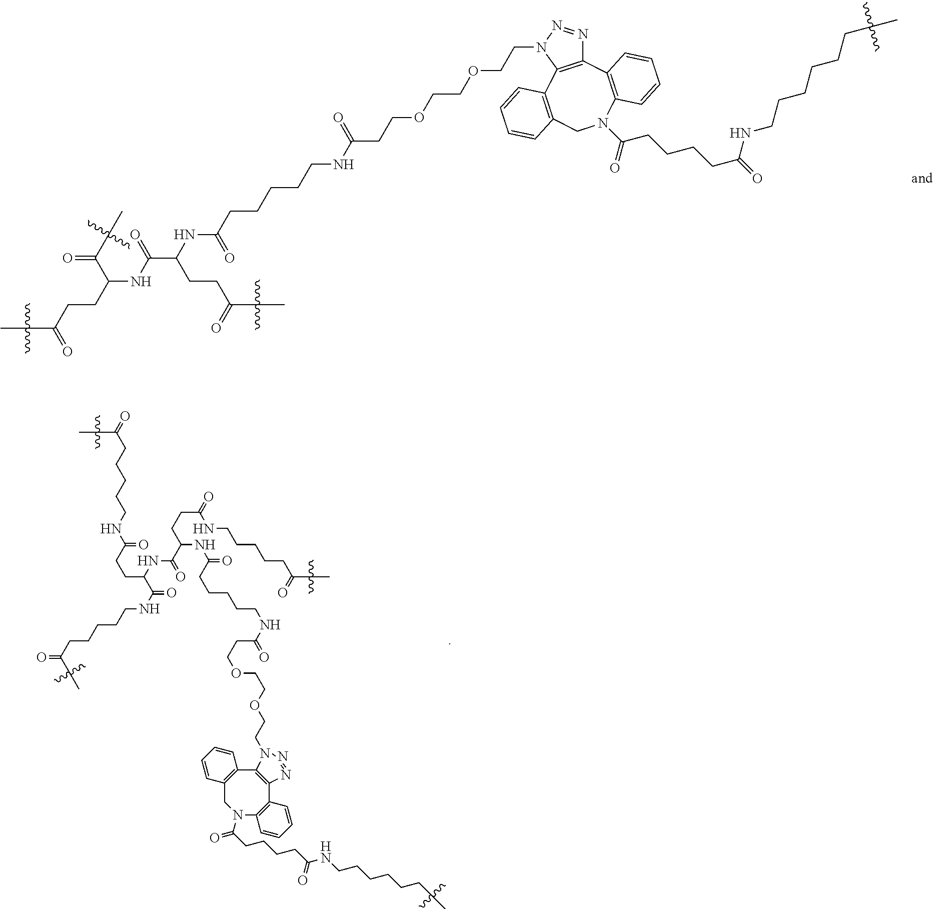 [SMILES: *CCCCCCNC(=O)CCCCC(=O)N1Cc2ccccc2-c2c(nnn2CCOCCOCCC(=O)NCCCCCC(=O)NC(CCC(*)=O)C(=O)NC(CCC(*)=O)C(*)=O)-c2ccccc21 ; *CCCCCCNC(=O)CCCCC(=O)N1Cc2ccccc2-c2c(nnn2CCOCCOCCC(=O)NCCCCCC(=O)NC(CCC(=O)NCCCCCC(*)=O)C(=O)NC(CCC(=O)NCCCCCC(*)=O)C(=O)NCCCCCC(*)=O)-c2ccccc21]

E120. The compound of E101 or E102, which is selected from the group consisting of:

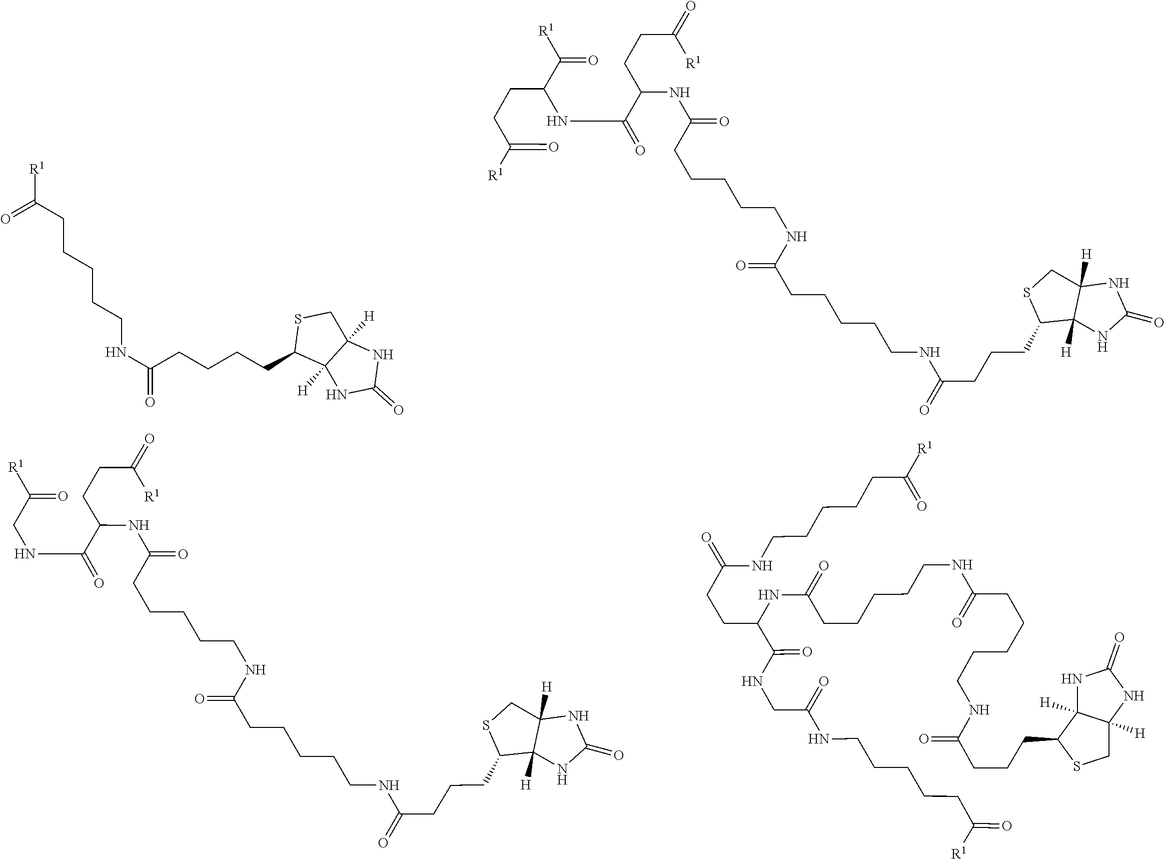

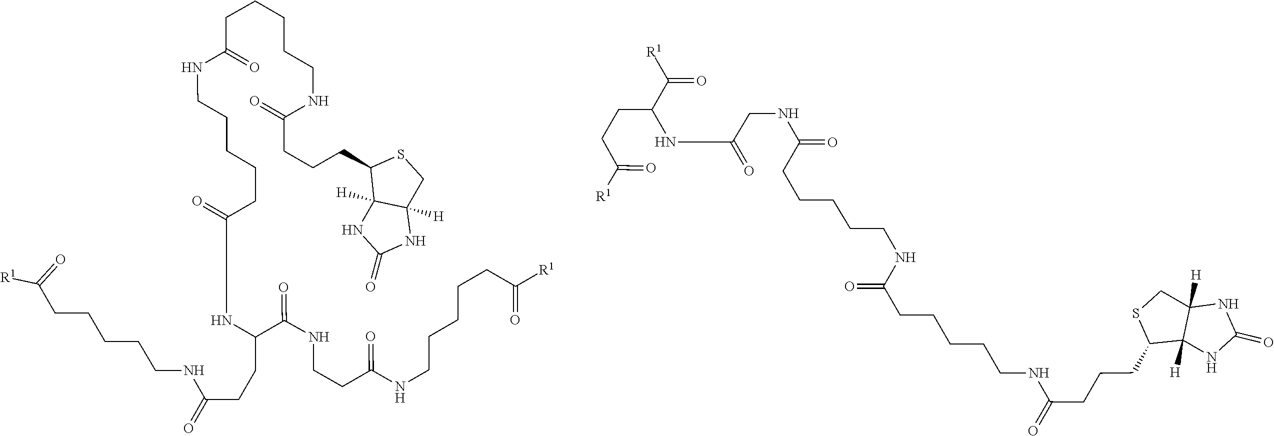

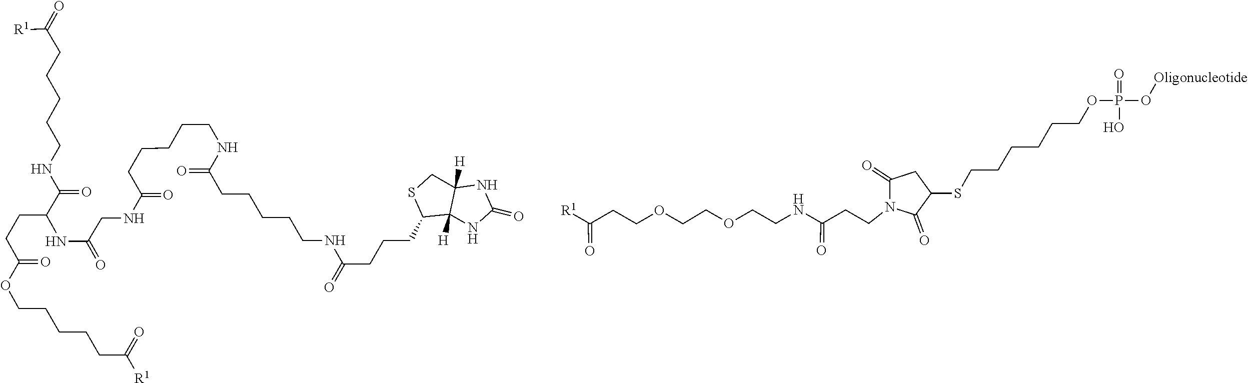

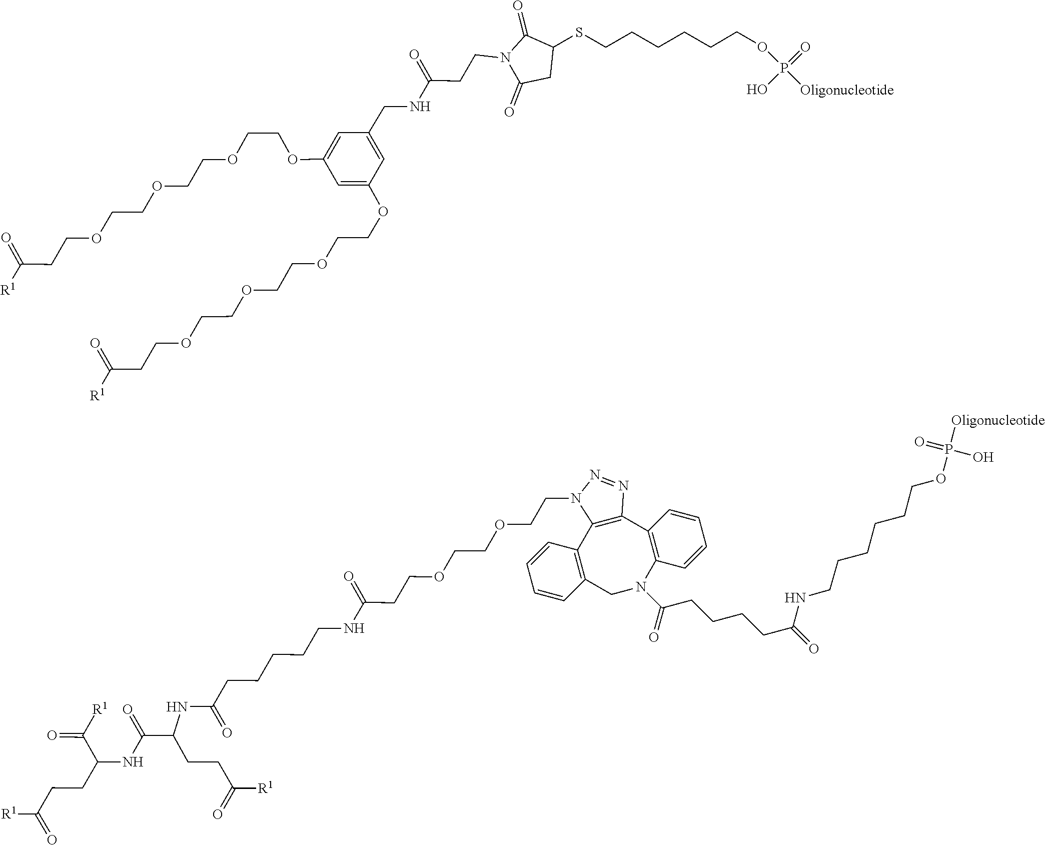

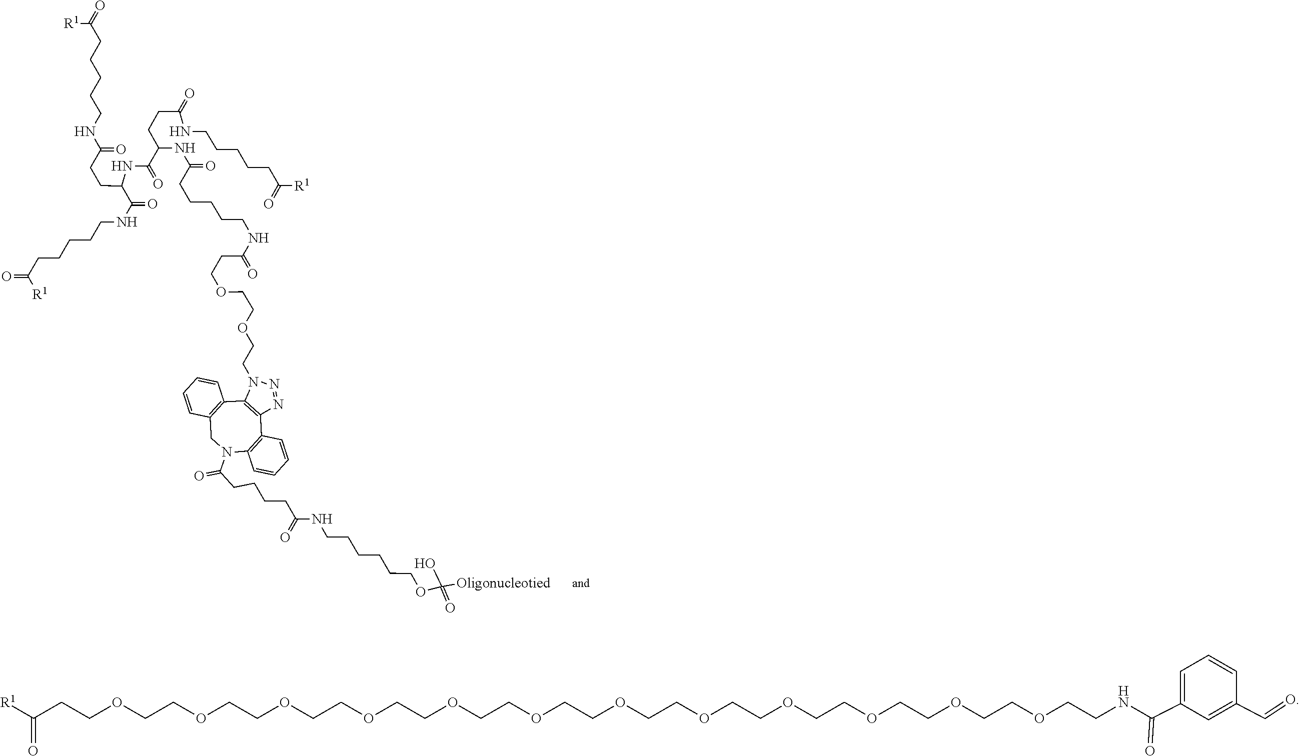

E121. The compound or salt of E101, wherein y is 1 and R² is an endosomal release polymer of formula XX:

-[PEGMAₘ-M²ₙ]ᵥ-[DMAEMA_{q}-PAAᵣ-BMAₛ]_{w} (XX)

wherein:
PEGMA is polyethyleneglycol methacrylate residue with 2-20 ethylene glycol units; M² is a methacrylate residue selected from the group consisting of
   a (C₄-C₁₈)alkyl-methacrylate residue;
   a (C₄-C₁₈)branched alkyl-methacrylate residue;
   a cholesteryl methacrylate residue;
   a (C₄-C₁₈)alkyl-methacrylate residue substituted with one or more fluorine atoms; and
   a (C₄-C₁₈)branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa; and w is 1 to 25 kDa.

E122. The compound or salt of E121, which is a compound of the following formula:

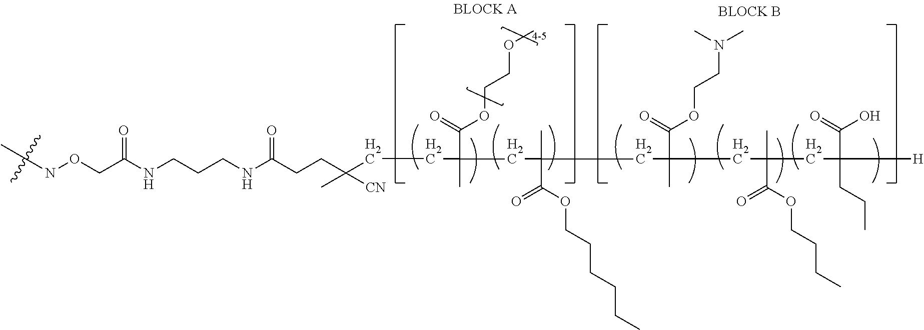

or a salt thereof. Stoichiometry (m and n; q, r and s) and molecular weights (v and w) are as defined in E121.

### Processes of the Invention

In one embodiment, the invention provides a process for preparing a diblock polymer of formula (X):

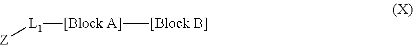

Prieve et al (Mol Ther 2018) describe the synthesis of di-block polymers using RAFT polymerisation techniques, wherein the alpha end of the polymer features either a mannose or GalNac monosaccharide ligand. Details were limited, input and output amounts were not recorded, and no yields were given. When preparing analogous GalNAc polymers, the synthesis can require significant amounts of input starting material chain transfer agent (CTA) relative to the amount of output polymer. For example, in a synthesis using GalNAc CTA, 180 g of input CTA was utilised to produce approximately 50 g of final polymer. This ratio of starting material to product seems to be consistent irrespective of the scale of the synthesis. In cases where ligands are relatively cheap and accessible, this material requirement is not necessarily an obstacle. However, in cases where the ligand may be significantly more expensive, this high input material requirement becomes prohibitive.

In addition, although RAFT polymerisation methodology is reported to be tolerant of a range of functional groups, it does not necessarily extend to all possible functional groups, including the functionality that may be present in a peptide where each amino acid residue may have a different potentially reactive group in its side chain. Incorporating protection group strategies to address the attendant complications would add significant technical complexity to the synthesis and likely further increase the material requirement and cost associated with trying to execute RAFT polymerisation on ligands more complicated than monosaccharides.

One possible solution to address these shortcomings would be to couple the ligand after polymerisation has been conducted. This would simultaneously reduce the amount of required ligand significantly (as the MW of the ligand is usually approximately 10% of the polymer MW) and it would also mitigate or eliminate the need for protection group strategies for complex ligands. The choice for conjugation strategy is not straightforward. The chemistry must be mild, and easy to execute, essentially quantitative, and the resultant functionality must be relatively biologically inert (not subject to in-vivo metabolism, or reactive with proteins and genetic material). It is also necessary to avoid pronounced sizes and structures that may deleteriously affect polymeric hydrophilic/hydrophobic balance and consequent micelle formation. These criteria significantly limit the available choices.

Conjunction via oxime formation is one possible choice. Indeed, oxime formation proceeded to an appreciable degree particularly in the presence of an acid catalyst. (e.g trifluoroacetic acid (TFA)) Thus, oxime chemistry is particularly suitable for ligands (functional and protecting groups) that are not acid sensitive.

For example, while GalNAc, is conjugatable to polymer via processes known in the art, and would be less suitable to be conjugated in the new process according to the present invention because it might undergo acid catalyzed hydrolysis at the glycometric centre resulting in ligand elimination, there are a number of ligands such as those described herein including the peptides described herein, which are particularly suitable to be conjugated by the process according to the present invention, but cannot be conjugated to a satisfying degree by the processes previously known in the art.

Further to this, polymer synthesis is complicated by the lack of useful, non destructive, diagnostic techniques for reaction monitoring and determination of reaction completion. The processes of the invention enable accurate determination of reaction endpoint, confirmation of ligand attachment, and they provide an approach to reaction clean up that removes unwanted reagents from the reaction without the need for additional precipitation or dialysis that can significantly impact recovered yield of the final polymer.

As used herein, the term “block copolymer” refers to two or more homopolymer or copolymer subunits linked by covalent bonds. Block copolymers with two or three distinct blocks are called diblock copolymers and triblock copolymers, respectively. A schematic generalization of a diblock copolymer is represented by the formula [F;G,Hh . . . ]g-[JjKxLi . . . ]., wherein each letter stands for a constitutional unit derived from polymerization of a corresponding monomer and wherein each subscript to a constitutional unit represents the mole fraction of that unit in the particular block, the three dots indicate that there may be more (there may also be fewer) constitutional units in each block and q and r indicate the molecular weight of each block in the diblock copolymer. As suggested by the schematic, in some instances, the number and the nature of each constitutional unit is separately controlled for each block. The schematic is not meant and should not be construed to infer any relationship whatsoever between the number of constitutional units and the number of different types of constitutional units in each of the blocks. Nor is the schematic meant to describe any particular number or arrangement of the constitutional units within a particular block. In each block the constitutional units may be disposed in a purely random, an alternating random, a regular alternating, a regular block or a random block configuration unless expressly stated to be otherwise. A purely random configuration, for example, may have the non-limiting form: f-f-g-h-f-g-g-h-g-h-h-h . . . . An exemplary alternating random configuration may have the non-limiting form: f-g-f-h-g-f-g-h-g-f-h . . . , and an exemplary regular alternating configuration may have the non-limiting form: f-g-h-fg-h-f-g-h . . . . An exemplary regular block configuration may have the following non-limiting configuration: . . . f-f-f-g-g-g-h-h-h-f-f . . . , while an exemplary random block configuration may have the non-limiting configuration: f-f-f-h-h-f-f-g-g-g-h-h-h-f-f-h-h-h . . . . In a gradient polymer, the content of one or more monomeric units increases or decreases in a gradient manner from the a-end of the polymer to the w-end. In none of the preceding generic examples is the particular juxtaposition of individual constitutional units or blocks or the number of constitutional units in a block or the number of blocks meant nor should they be construed as in any manner bearing on or limiting the actual structure of block copolymers described herein. As used herein, the brackets enclosing the constitutional units are not meant and are not to be construed to mean that the constitutional units themselves form blocks. That is, the constitutional units within the square brackets may combine in any manner with the other constitutional units within the block, i.e., purely random, alternating random, regular alternating, regular block or random block configurations. The block copolymers described herein are, optionally, alternate, gradient or random block copolymers. Block copolymers and methods for their preparation are described in International Patent Application Publication Number WO2015/017519, which is hereby incorporated herein by reference in its entirety.

However, according to the present invention, the ligand (such as e.g. targeting ligand), is not incorporated during polymerization such as contemplated in WO2015/017519, e.g. by incorporating monomer A4 (comprising a targeting moiety T2) into block A of the polymer, but rather, according to the present invention, the ligand T (e.g. targeting ligand) is incorporated post polymerization. This allows, among other aspects, to control for stoichiometry of ligand incorporated per co-polymer, and also allows to incorporate ligands which are not susceptible to RAFT polymerization, such as in a non-limiting example oligonucleotides and peptides, such as the pPB peptide. In typical embodiments, one ligand (T), or one moiety bearing a natural number of targeting ligands (T) is being incorporated into every one polymer, i.e. according to the present invention a typical stoichiometry is 1, or a multiplicity thereof. The process of the present invention is thus differentiated over WO2015/017519 at least in higher specificity of ligand incorporation and in allowing incorporating certain ligands that the process of WO2015/017519 does not allow for. A further advantage of the present invention results from the fact that the ligand T is always placed on the hydrophilic end of the polymer, as opposed to incorporation at a random position as taught in WO2015/017519. In other words, the ligand endosome release polymer (ERT) of the present invention, which can be visualized by Formula (I}:

(R¹)ₓ-L-(R²)_{y} (I)

wherein R¹ and R² and L are defined as specified herein, and wherein x is selected from an integer among 1, 2, 3, 4 or 5, and y is selected from an integer among 1, 2, 3, 4 or 5, provides for a defined stoichiometry between R¹ and R².

Examples of block copolymers according to the present invention include those where Block A is a first block that is a random copolymer formed from monomers of formulae A1, A2 and A3 as described herein. Additional examples of block copolymers include those where Block A is a first block that is a random copolymer formed from monomers comprising formulae A1 and A2 as described above. Additional examples of block copolymers include those where A is a first block that is a polymer formed from monomer A2 as described above. Additional examples of block copolymers include those where Block A is a first block that is a random copolymer comprising residues of monomers of formula A1. Additional examples of block copolymers include those where Block A is a first block that is a random copolymer comprising residues of monomers of formula A2. Additional examples of block copolymers include those where Block A is a first block that is a random copolymer comprising residues of monomers of formula A3.

Examples of block copolymers include those where Block B is a second block that is a random copolymer formed from monomers of formulae B1, B2 and B3 as described herein. Additional examples of block copolymers include those where Block B is a second block that is a random copolymer comprising residues of monomers of formula B1. Additional examples of block copolymers include those where Block B is a second block that is a random copolymer comprising residues of monomers of formula B2. Additional examples of block copolymers include those where Block B is a second block that is a random copolymer comprising residues of monomers of formula B3.

In the diblock polymer of formula (X and XI), Block A comprises residues derived from the polymerization of monomers of formulae A1 and A2. As used herein, the term “residue” means the portion of the monomer that remains in the polymer following polymerization. In one embodiment, Block A comprises one or more residues of monomers A1 and A2 and has a molecular weight of from about 1 kDa to about 25 kDa. In another embodiment, Block A comprises one or more residues of monomers A1 and A2 and has a molecular weight of from about 2 kDa to about 20 kDa. In another embodiment, Block A comprises one or more residues of monomers A1 and A2 and has a molecular weight of from about 2 kDa to about 15 kDa. In another embodiment, Block A comprises one or more residues of monomers A1 and A2 and has a molecular weight of from about 2 kDa to about 10 kDa. In another embodiment, Block A comprises one or more residues of monomers A1 and A2 and has a molecular weight of from about 2 kDa to about 5 kDa. In another embodiment, Block A comprises one or more residues of monomers A1 and A2 and has a molecular weight of from about 5 kDa to about 20 kDa. In another embodiment, Block A comprises one or more residues of monomers A1 and A2 and has a molecular weight of from about 5 kDa to about 15 kDa. In another embodiment, Block A comprises one or more residues of monomers A1 and A2 and has a molecular weight of from about 5 kDa to about 10 kDa. In another embodiment, Block A comprises one or more residues of monomers A1 and A2 and has a molecular weight of from about 10 kDa to about 25 kDa. In another embodiment, Block A comprises one or more residues of monomers A1 and A2 and has a molecular weight of from about 10 kDa to about 20 kDa. In another embodiment, Block A comprises one or more residues of monomers A1 and A2 and has a molecular weight of from about 10 kDa to about 15 kDa.

In the diblock polymer of formula (X and XI), Block B comprises residues derived from the polymerization of monomers of formulae B1, B2 and B3. As used herein, the term “residue” means the portion of the monomer that remains in the polymer following polymerization. In one embodiment, Block B comprises one or more residues of monomers B1, B2 and B3 and has a molecular weight of from about 1 kDa to about 25 kDa. In another embodiment, Block B comprises one or more residues of monomers B1, B2 and B3 and has a molecular weight of from about 2 kDa to about 20 kDa. In another embodiment, Block B comprises one or more residues of monomers B1, B2 and B3 and has a molecular weight of from about 2 kDa to about 15 kDa. In another embodiment, Block B comprises one or more residues of monomers B1, B2 and B3 and has a molecular weight of from about 2 kDa to about 10 kDa. In another embodiment, Block B comprises one or more residues of monomers B1, B2 and B3 and has a molecular weight of from about 2 kDa to about 5 kDa. In another embodiment, Block B comprises one or more residues of monomers B1, B2 and B3 and has a molecular weight of from about 5 kDa to about 20 kDa. In another embodiment, Block B comprises one or more residues of monomers B1, B2 and B3 and has a molecular weight of from about 5 kDa to about 15 kDa. In another embodiment, Block B comprises one or more residues of monomers B1, B2 and B3 and has a molecular weight of from about 5 kDa to about 10 kDa. In another embodiment, Block B comprises one or more residues of monomers B1, B2 and B3 and has a molecular weight of from about 10 kDa to about 25 kDa. In another embodiment, Block B comprises one or more residues of monomers B1, B2 and B3 and has a molecular weight of from about 10 kDa to about 20 kDa. In another embodiment, Block B comprises one or more residues of monomers B1, B2 and B3 and has a molecular weight of from about 10 kDa to about 15 kDa.

According to the processes of the invention, Z and Y are selected so that Z in the diblock polymer of formula (X and XI) will react with Y in a compound of formula (XII):

T-L₂-Y (XII)

to form a group Y′Z′ and yield a conjugate of formula:

T-L₂-Y′Z′-L₁-[Block A]-[Block B]

In one embodiment, the conjugate is a conjugate of formula (XIII):

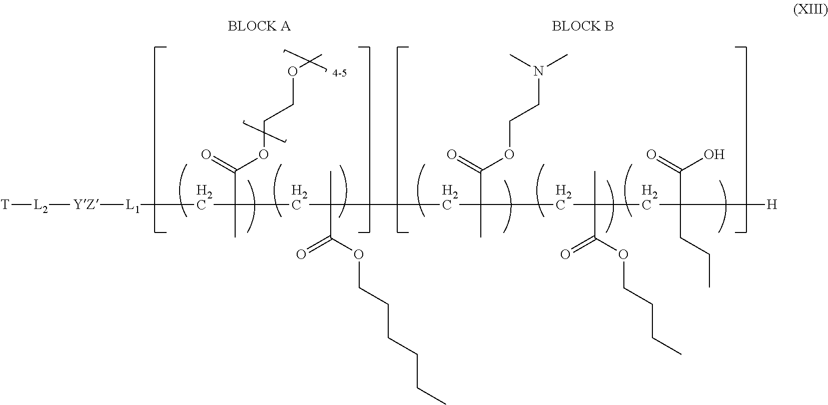

Suitable non-limiting examples of Z and Y as well as the corresponding groups Y′Z′ that remain in the conjugate of formula (XIII) after the reaction of Y and Z are illustrated in the following table A

TABLE A
Embodiments for X and Y, and the resulting conjugation product X′-Y′ according to certain exemplified embodiments
of the invention.

| Z | Y | Z′Y′ |
|---|---|---|
| 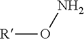 | 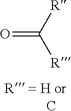 | 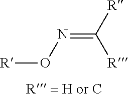 |
| 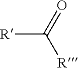 | 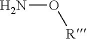 | 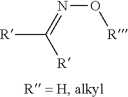 |
| R′—N₃ | ≡—R′′ | 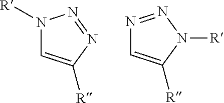 |
| R′—≡ | N₃—R′′ | 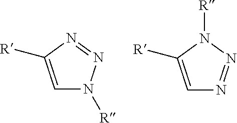 |
| 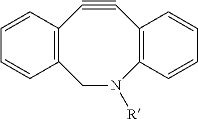 | N₃—R′′ | 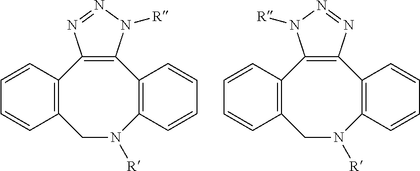 |
| R′—N₃ | 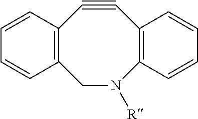 | 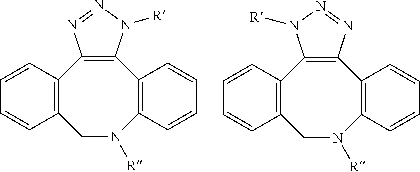 |

In the first row of the table, the aminoxy of Z can react with an aldehyde (R′″ is H) or with a ketone (R′″ is C) of Y to provide the corresponding Z′Y′ oxime. In the second row of the table, the aminoxy of Y can react with an aldehyde (R″ is H) or with a ketone (R″ is C) of Z to provide the corresponding Z′Y′ oxime.

It may be desirable to prepare a compound of formula (X) wherein Z is protected with a protecting group. In such a case, the protecting group may be removed from Z prior to reaction with the compound of formula (XII). It may also be desirable to prepare a compound of formula (XII) wherein Y is protected with a protecting group. In such a case, the protecting group may be removed from Y prior to reaction with the compound of formula (X). A compound of formula (X) wherein Z is protected with a protecting group is a useful intermediate for preparing a conjugate of the invention (e.g., a conjugate of formula (XIII)). Such a protected compound of formula (X) is an embodiment of the invention. A compound of formula (XII) wherein Y is protected with a protecting group is a useful intermediate for preparing a conjugate of the invention (e.g., a conjugate of formula (XIII)). Such a protected compound of formula (XII) is an embodiment of the invention.

### EXAMPLES

The present invention will be described in greater detail by way of specific examples. The following examples are offered for illustrative purposes and are not intended to limit the invention in any manner. Those of skill in the will readily recognize a variety of noncritical parameters which can be changed or modified to yield essentially the same result.

### Nomenclature

pPB=(N- to C-terminus) H2N—CSNLIDC—COOH (disulphide between two Cys). Scrambled pPB=(N- to C-terminus) H2N-CIDNLSRC—COOH) (disulphide between two Cys). Used as a control for the pPB active ligand.

### Example 1: Biotinylated Ligands

Biotinylated versions of ligands were prepared for in vitro binding experiments, to gauge relative binding efficiencies (see Example 6, 7 and 8). The following biotinylated ligands were prepared using standard solid phase peptide synthesis (SPPS). Biotin was coupled using standard organic chemistry amide coupling techniques and the final ligands were purified with reverse phase HPLC. Product confirmation was achieved with mass spectrometry and product purity determined by RPLC techniques.

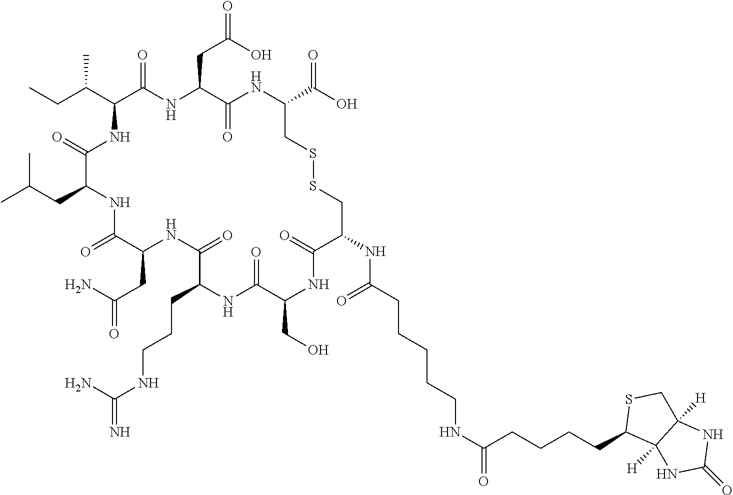 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCCCCNC(=O)CCCC[C@H]2SC[C@H]3NC(=O)N[C@H]32)CSSC[C@@H](C(=O)O)NC(=O)[C@H](CC(=O)O)NC1=O]

pPB monovalent Biotin (Compound 1). MW: Calc 1260.56. Found 1260.2. Purity (HPLC) >90%.

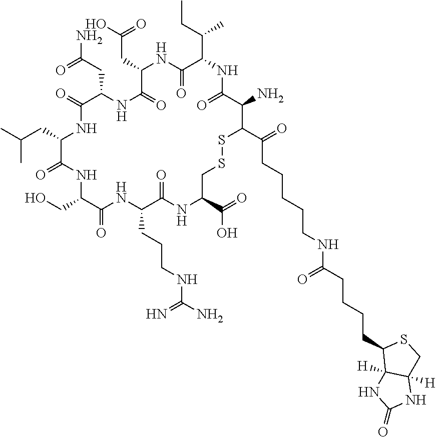 [SMILES: CC[C@H](C)[C@@H]1NC(=O)[C@@H](N)C(C(=O)CCCCCNC(=O)CCCC[C@H]2SC[C@H]3NC(=O)N[C@H]32)SSC[C@@H](C(=O)O)NC(=O)[C@H](CCCNC(=N)N)NC(=O)[C@H](CO)NC(=O)[C@H](CC(C)C)NC(=O)[C@H](CC(N)=O)NC(=O)[C@H](CC(=O)O)NC1=O]

Scrambled pPB monovalent Biotin. (Compound 2). MW: Calc 1260.56. found 1260.2. Purity (HPLC >90%

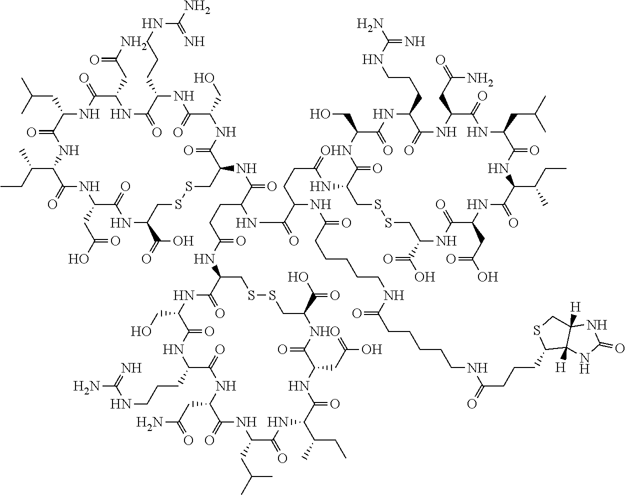

pPB Trivalent Biotin (Compound 3). MW: Calc. 3437.97. Found 3438.89. Purity (HPLC) >90%.

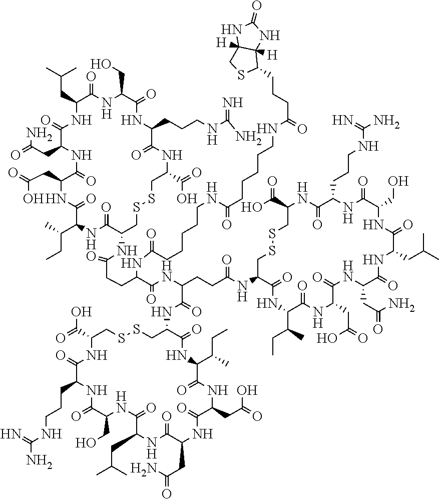

Scrambled pPB Trivalent Biotin (Compound 4). MW: Calc. 3438.97. Found 3438.39. Purity (HPLC) >90%

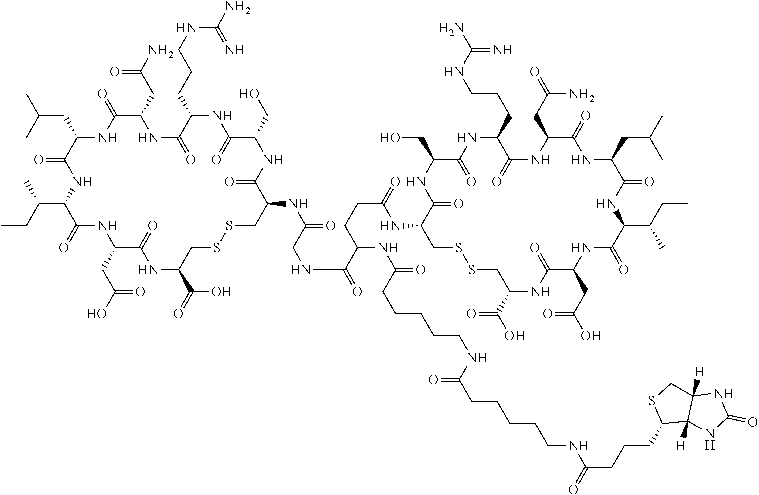 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCC(NC(=O)CCCCCNC(=O)CCCCCNC(=O)CCC[C@@H]2SC[C@@H]3NC(=O)N[C@@H]32)C(=O)NCC(=O)N[C@H]2CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC2=O)CSSC[C@@H](C(=O)O)NC(=O)[C@H](CC(=O)O)NC1=O]

pPB Divalent Biotin “1A” (Compound 5). MW: Calc. 2462.87. found 2463.37. Purity (HPLC >90%).

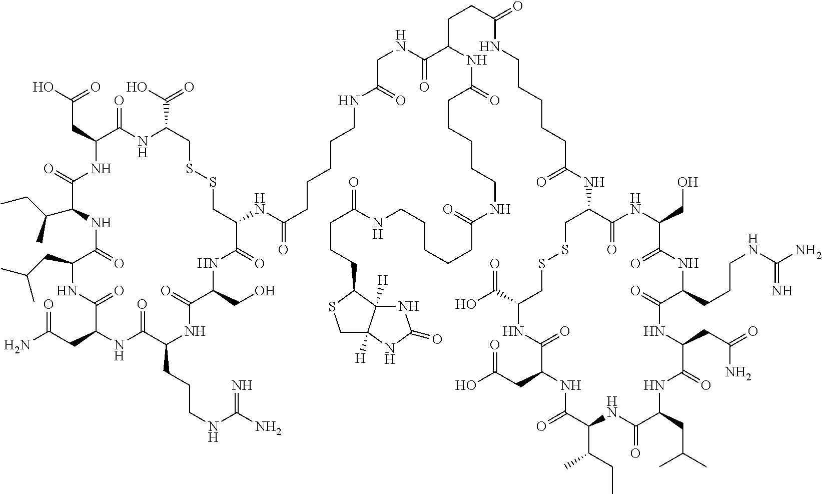 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCCCCNC(=O)CCC(NC(=O)CCCCCNC(=O)CCCCCNC(=O)CCC[C@@H]2SC[C@@H]3NC(=O)N[C@@H]32)C(=O)NCC(=O)NCCCCCC(=O)N[C@H]2CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC2=O)CSSC[C@@H](C(=O)O)NC(=O)[C@H](CC(=O)O)NC1=O]

pPB Divalent Biotin “in” (Compound 6). MW: Calc. 2689.18. Found 2690.32. Purity (HPLC >90% o)

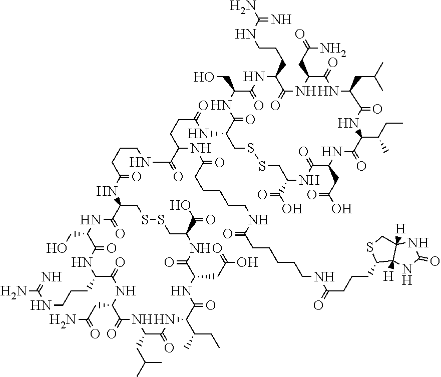 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCCNC(=O)C(CCC(=O)N[C@H]2CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC2=O)NC(=O)CCCCCNC(=O)CCCCCNC(=O)CCC[C@@H]2SC[C@@H]3NC(=O)N[C@@H]32)CSSC[C@@H](C(=O)O)NC(=O)[C@H](CC(=O)O)NC1=O]

pPB Divalent “2A” (Compound 7). MW: Calc 2490.92. Found 2491.2. Purity (HPLC >90%).

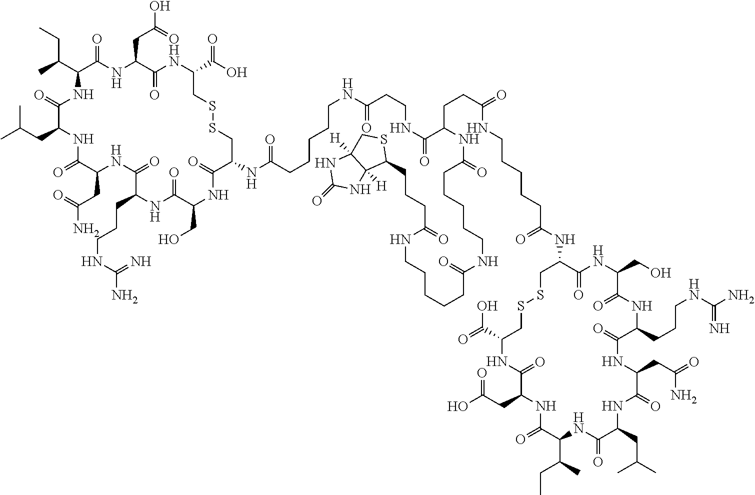 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCCCCNC(=O)CCNC(=O)C(CCC(=O)NCCCCCC(=O)N[C@H]2CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC2=O)NC(=O)CCCCCNC(=O)CCCCCNC(=O)CCC[C@@H]2SC[C@@H]3NC(=O)N[C@@H]32)CSSC[C@@H](C(=O)O)NC(=O)[C@H](CC(=O)O)NC1=O]

pPB Divalent “2B” (Compound 8). MW: Calc 2717.24. Found 2718.15. Purity (HPLC >90%)

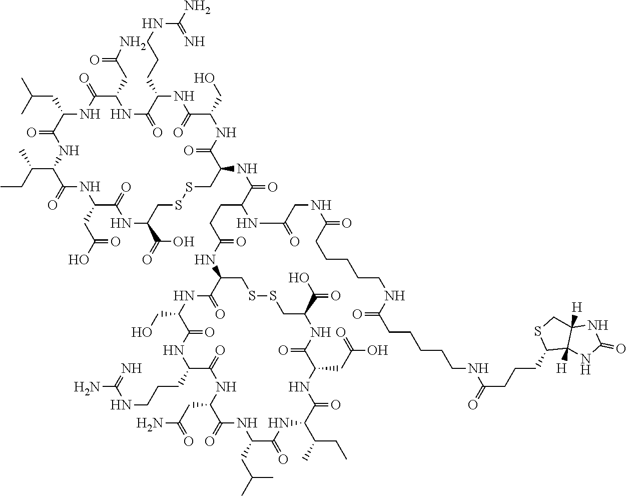 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCC(NC(=O)CNC(=O)CCCCCNC(=O)CCCCCNC(=O)CCC[C@@H]2SC[C@@H]3NC(=O)N[C@@H]32)C(=O)N[C@H]2CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC2=O)CSSC[C@@H](C(=O)O)NC(=O)[C@H](CC(=O)O)NC1=O]

pPB Divalent “2C” (Compound 9). MW: Calc 2405.82. Found 2406.22. Purity (HPLC >90%).

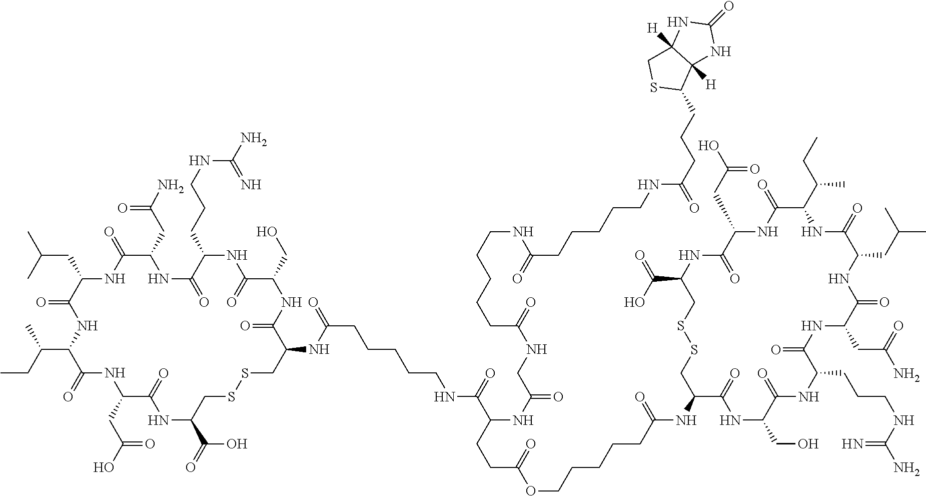 [SMILES: CC[C@H](C)[C@@H]1NC(=O)[C@H](CC(C)C)NC(=O)[C@H](CC(N)=O)NC(=O)[C@H](CCCNC(=N)N)NC(=O)[C@H](CO)NC(=O)[C@@H](NC(=O)CCCCCNC(=O)C(CCC(=O)OCCCCCC(=O)N[C@H]2CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC2=O)NC(=O)CNC(=O)CCCCCNC(=O)CCCCCNC(=O)CCC[C@@H]2SC[C@@H]3NC(=O)N[C@@H]32)CSSC[C@@H](C(=O)O)NC(=O)[C@H](CC(=O)O)NC1=O]

pPB Divalent “3B” (Compound 10). MW: Calc 2632.13. Found 2632.54. Purity (HPLC >90%)

### Example 2: Synthesis of Peptide Conjugated PEG 12 Benzaldehydes

The following pPB benzaldehyde ligands (18a and 18b) were synthesized to allow for conjugation to the endosome release polymer (27) once RAFT polymerization was completed.

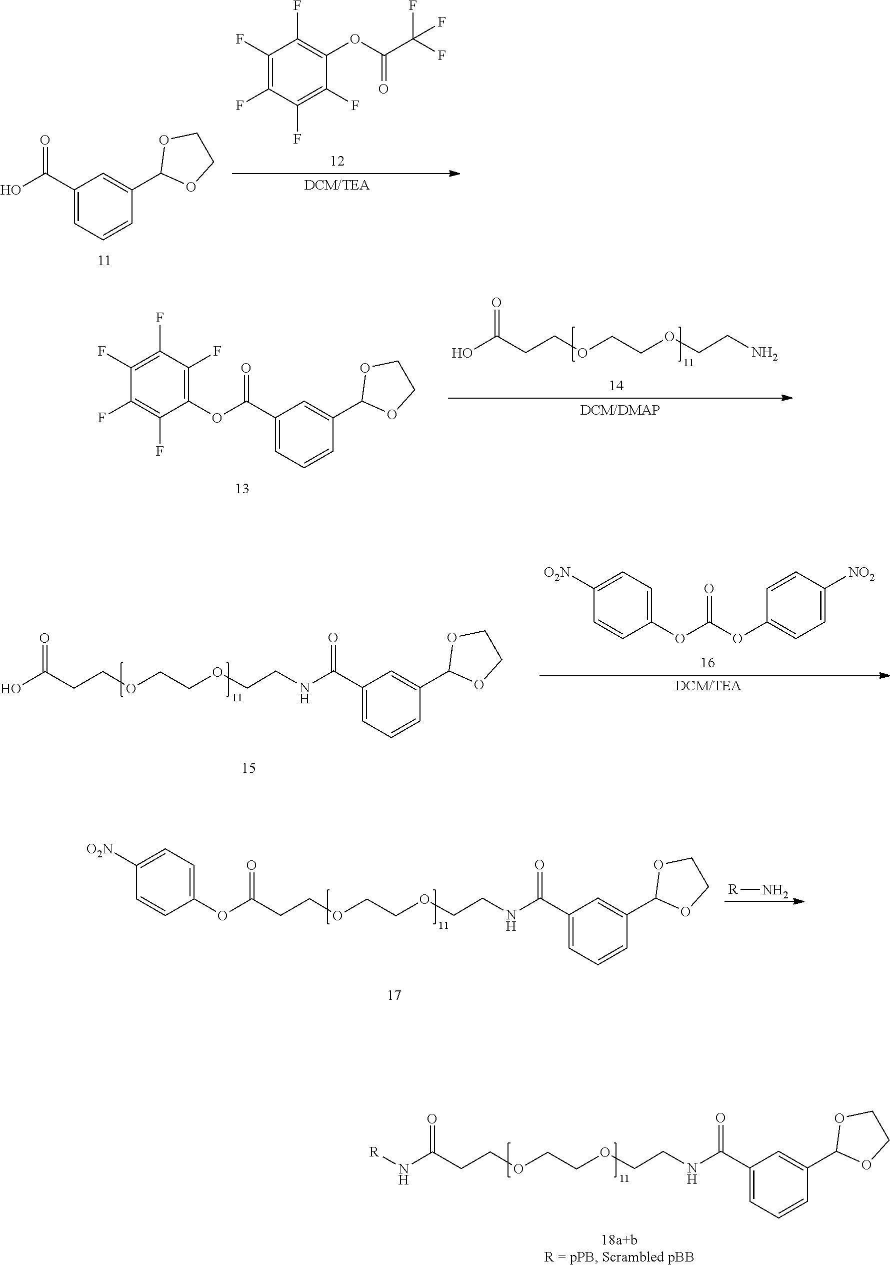

### a. Step 1 Synthesis of perfluorophenyl 3-(1,3-dioxolan-2-yl)benzoate (13)

3-(1,3-Dioxolan-2-yl)benzoic acid (11) (7.9 g, 41 mmol) and (TEA 11.4 mL, 81 mmol) were stirred in DCM at 0 °C. 2,3,4,5,6-Pentafluorophenyl 2,2,2-trifluoroacetate (12) (7.0 mL, 41 mmol) was added dropwise and the reaction allowed to warm to RT over 2 hr. The reaction was concentrated in-vacuo, taken up in EtOAc (200 mL) and washed with NaHCO₃ (2×100 mL) and brine (1×100 mL), dried (MgSO₄), filtered and concentrated in-vacuo to give perfluorophenyl 3-(1,3-dioxolan-2-yl)benzoate (13) (13.6 g, 92.8%) which was used without purification.

### b. Step 2 Synthesis of 1-{[3-(1,3-dioxolan-2-yl)phenyl]formamido}-3,6,9,12,15,18,21,24,27,30,33,36-dodecaoxanonatriacontan-39-oic acid (15)

1-Amino-3,6,9,12,15,18,21,24,27,30,33,36-dodecaoxanonatriacontan-39-oic acid (14) (7 g, 11.3 mmol), 2,3,4,5,6-pentafluorophenyl 3-(1,3-dioxolan-2-yl)benzoate (13) (4.5 g, 12.5 mmol), and DMAP (69 mg, 0.6 mmol) were stirred at in DCM (200 ml) at RT for 16 hr. The reaction was concentrated in-vacuo and purified by automated flash chromatography (0-10% MeOH/DCM) to give 1-{[3-(1,3-dioxolan-2-yl)phenyl]formamido}-3,6,9,12,15,18,21,24,27,30,33,36-dodecaoxanonatriacontan-39-oic acid (15) (5.1 g, 56.7%).

### c. Step 3 Synthesis of 4-nitrophenyl 1-{[3-(1,3-dioxolan-2-yl)phenyl]formamido}-3,6,9,12,15,18,21,24,27,30,33,36-dodecaoxanonatriacontan-39-oate (17)

1-{[3-(1,3-dioxolan-2-yl)phenyl]formamido}-3,6,9,12,15,18,21,24,27,30,33,36-dodecaoxanonatriacontan-39-oic acid (15) (5.1 g, 6.4 mmol), TEA (1.4 mL, 9.6 mmol) and bis(4-nitrophenyl) carbonate (16) (2.15 g, 7.1 mmol) were stirred in DCM (100 mL) at RT for 16 hr. The reaction was diluted with DCM (100 mL), washed with saturated NaHCO₃ (5×75 mL), concentrated in-vacuo and the residue purified by automated flash chromatography (0-10% MeOH/DCM) to give 4-nitrophenyl 1-{[3-(1,3-dioxolan-2-yl)phenyl]formamido}-3,6,9,12,15,18,21,24,27,30,33,36-dodecaoxanonatriacontan-39-oate (17) (5.1 g, 86.8%).

### d. Step 4 Synthesis of pPB PEG 12 benzaldehydes (18 a +b)

pPB and scrambled pPB peptides were prepared using standard solid phase peptide synthesis techniques. The peptides were globally deprotected followed by amide conjugation with activated carboxylic acid (n). deprotection of the dioxalan, purification by reverse phase RPLC and subsequent lyophilization gave peptide conjugated PEG 12 benzaldehydes (18 a +b).

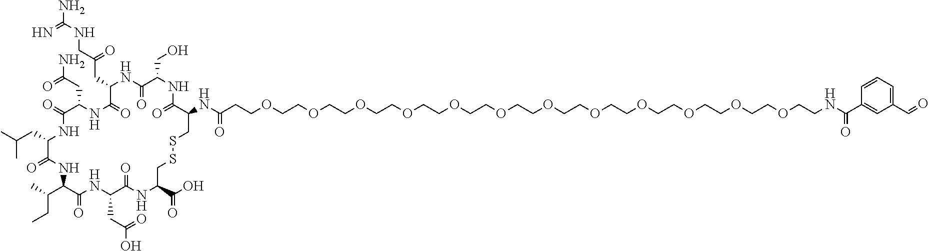 [SMILES: CC[C@H](C)[C@H]1NC(=O)[C@H](CC(C)C)NC(=O)[C@H](CC(N)=O)NC(=O)[C@H](CC(=O)CNC(=N)N)NC(=O)[C@H](CO)NC(=O)[C@@H](NC(=O)CCOCCOCCOCCOCCOCCOCCOCCOCCOCCOCCOCCOCCNC(=O)c2cccc(C=O)c2)CSSC[C@@H](C(=O)O)NC(=O)[C@H](CC(=O)O)NC1=O]

Monovalent pPB PEG12 benzaldehyde (18a). MW: Calc. 1652.92 Found 1653.92. Purity (RP-HPLC) >95%.

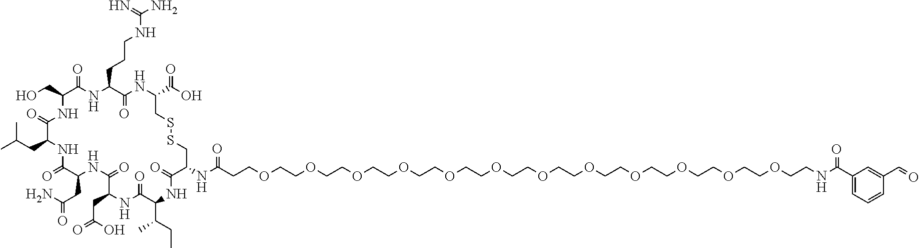 [SMILES: CC[C@H](C)[C@@H]1NC(=O)[C@@H](NC(=O)CCOCCOCCOCCOCCOCCOCCOCCOCCOCCOCCOCCOCCNC(=O)c2cccc(C=O)c2)CSSC[C@@H](C(=O)O)NC(=O)[C@H](CCCNC(=N)N)NC(=O)[C@H](CO)NC(=O)[C@H](CC(C)C)NC(=O)[C@H](CC(N)=O)NC(=O)[C@H](CC(=O)O)NC1=O]

Scrambled monovalent pPB PEG12 benzaldehyde (18b). MW: Calc. 1652.92 Found 1653.85. Purity (RP-HPLC) >95%

### Example 3: Synthesis of Endosomal Release Polymer

The endosome release polymer was synthesized using RAFT polymerization and terminated with a protected aminooxy functional group to allow for subsequent oxime conjugation to benzaldehyde peptide ligands (18a and 18b).

### Part 1) Synthesis of Chain Transfer Agent tert-butyl ((6-cyano-6-methyl-9,15-dioxo-4-thioxo-3,5-dithia-10,14-diazahexadecan-16-yl)oxy)carbamate (compound 24)

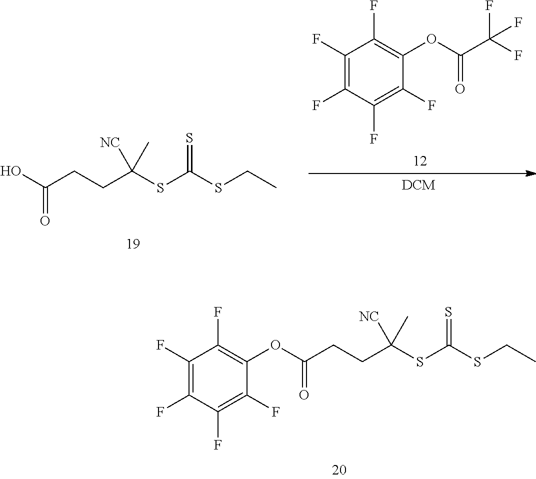 [SMILES: CCSC(=S)SC(C)(C#N)CCC(=O)O ; O=C(Oc1c(F)c(F)c(F)c(F)c1F)C(F)(F)F ; CCSC(=S)SC(C)(C#N)CCC(=O)Oc1c(F)c(F)c(F)c(F)c1F]

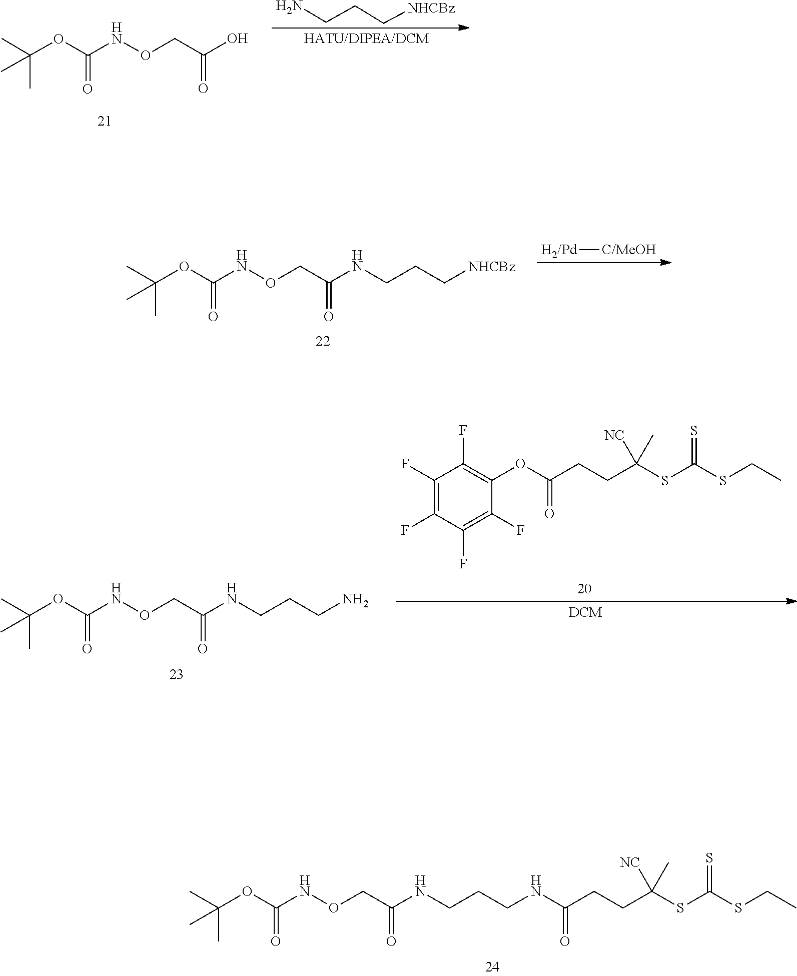

### a. Step 1 Synthesis of perfluorophenyl 4-cyano-4-(((ethylthio)carbonothioyl)thio)-pentanoate (20)

4-Cyano-4-{[(ethylsulfanyl)methanethioyl]sulfanyl}-4-methylbutanoic acid (19) (26 g, 99 mmol) and triethylamine (12 g, 118 mmol) were stirred at 0 °C in anhydrous DCM (500 mL). 2,3,4,5,6-Pentafluorophenyl 2,2,2-trifluoroacetate (12) (33 g, 118.5 mmol) was added slowly over 15 mins. The solution was stirred at 0 °C until complete. The reaction was washed with saturated NaHCO₃ (3×250 mL), dried (MgSO₄), filtered and concentrated in-vacuo to give perfluorophenyl 4-cyano-4-(((ethylthio)carbonothioyl)thio)pentanoate (20) (Quant) which was used without further purification.

### b. Step 2 Synthesis of tert-butyl (2-((3-(((benzyloxy)carbonyl)amino)propyl)amino)-2-oxoethoxy)carbamate (22)

{[(tert-butoxycarbonyl)amino]oxy}acetic acid (21) (20 g, 105 mmol), DIPEA (55 mL, 314 mmol), HATU (40 g, 105 mmol) and benzyl N-3-[amino]propylcarbamate HCl salt (26 g, 105 mmol) were stirred in DCM (350 mL) at RT for 16 h. DCM (150 mL) was added and the reaction washed with saturated NaHCO₃ and brine, dried (MgSO₄), concentrated in-vacuo and purified by automated flash chromatography (0-5% MeOH in DCM) to give tert-butyl N-{[(3-{[(benzyloxy)carbonyl]amino}propyl) carbamoyl]methoxy}carbamate (22)(31 g, 78.2%).

### c. Step 3 Synthesis of tert-butyl (2-((3-aminopropyl)amino)-2-oxoethoxy)-carbamate (23)

tert-Butyl N-{[(3-{[(benzyloxy)carbonyl]amino}propyl)carbamoyl]methoxy}-carbamate (22) (31 g, 81 mmol) was subjected to catalytic hydrogenation in MeOH (200 mL) over 10% palladium on carbon (cat) at RT for 16 hr. Upon completion the solution was filtered through celite and concentrated to dryness to give tert-butyl (2-((3-aminopropyl)amino)-2-oxoethoxy)carbamate (23) (19.4 g, 91.5%) which was used without further purification.

### d. Step 4 Synthesis of tert-butyl ((6-cyano-6-methyl-9,15-dioxo-4-thioxo-3,5-dithia-10,14-diazahexadecan-16-yl)oxy)carbamate (24)

tert-Butyl N-{[(3-aminopropyl)carbamoyl]methoxy}carbamate (23) (18 g, 73 mmol), 2,3,4,5,6-pentafluorophenyl 4-cyano-4-{[(ethylsulfanyl)methanethioyl]sulfanyl}-4-methylbutanoate (20) (31 g, 73 mmol) and DMAP (178 mg, 1.5 mmol) were stirred in DCM (100 mL) at RT until complete. The reaction was concentrated in-vacuo, taken up in EtOAc (250 mL), washed with sat'd NaHCO₃ (2×100 mL), dried (MgSO₄), filtered and concentrated in-vacuo. The residue was purified by automated flash chromatography (0-5% MeOH/DCM) to give tert-butyl ((6-cyano-6-methyl-9,15-dioxo-4-thioxo-3,5-dithia-10,14-diazahexadecan-16-yl)oxy)carbamate (24) (28 g, 78.1%). ¹H NMR (400 MHz, CDCl₃) δ 8.29 (bs, 1h), 7.79 (s, 1H), 6.62 (m, 1H), 5.29 (s, 2H), 4.32 (s, 2H), 3.27-3.39 (m, 6H), 2.40-2.50 (m, 4H), 1.88 (s, 3H), 1.72-1.85 (m, 3H), 1.48 (s, 9H), (1.34 (t, 3H).

### Part 2) Synthesis of “BOC-gly” Endosomal Release Polymer (Compound 25)

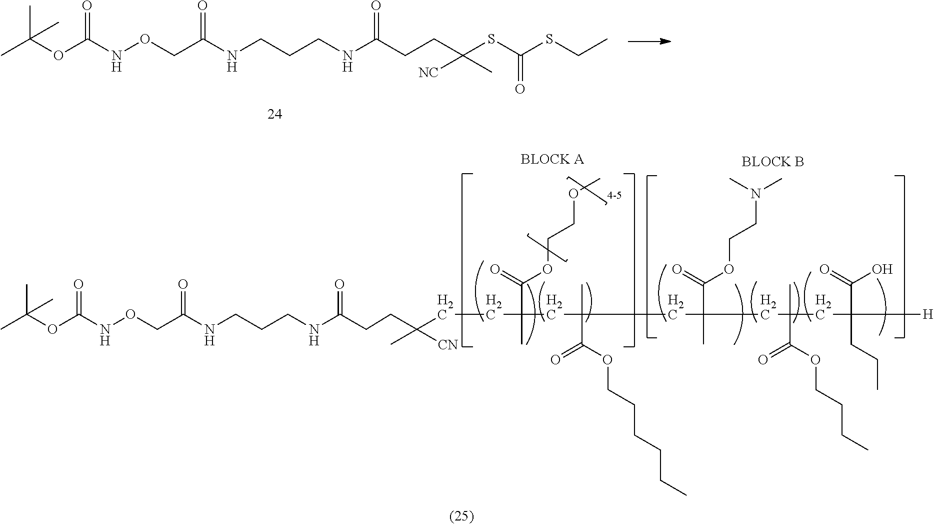

“BOC-Gly” endosomal release polymer (25) was prepared using RAFT polymerisation techniques from chain transfer agent (24) as with analogous synthetic techniques as described in Prieve, M. G., Harvie, P., Monahan, S. D., Roy, D., Li, A. G., Blevins, T. L., Paschal, A. E., Waldheim, M., Bell, E. C., Galperin, A., Ella-Menye, J. R., et al. (2018). Targeted mRNA Therapy for Ornithine Transcarbamylase Deficiency. Mol Ther 26, 801-813. 10.1016/j.ymthe.2017.12.024. The final released material had the following observed monomeric composition and average molecular weight.

| Step | Test | Method | Release Spec |
|---|---|---|---|
| Block 1 | Monomer Incorporation | HPLC | Pegma 75% (71-79%) |
| | | | HMA 25% (21-29%) |
| | Molecular Weight | Analytical GPC | 5.7 kDa ± 15% of target value (Mn 4.85-6.56 kDa) |
| | ¹H NMR | NMR | Consistent for structure, no indication of residual |
| | | | monomer |
| Block 2 | Monomer incorporation | HPLC | BMA 54% (47-57%) |
| | | | DMAEMA 35% (30-40%) |
| | | | PAA 12% (9-16%) |
| | Molecular Weight | Analytical GPC | 7.92 kDa ± 15% of target value (Mn 6.73-9.11 kDa); |
| | | | PDI < 1.6 |
| | ¹H NMR | NMR | Consistent for structure, no indication of residual |
| | | | monomer |
| CTA | Final polymer Molecular | Analytical GPC | ±15% of target value |
| Removal | Weight | | |
| | Removal of | UV | >98% |
| | Trithiocarbonate end | | |
| | group | | |

2′-O-Methyl nucleotides are depicted as “m”+UPPER CASE; 2′-Fluoro nucleotides are depicted as “f” +UPPER CASE; Phosphorothioate linkers are depicted as “s”.

### Part 3) Synthesis of Oxime Conjugated Ligand Functionalized Endosomal Release Polymers Exemplified by the Following General Reaction Protocol

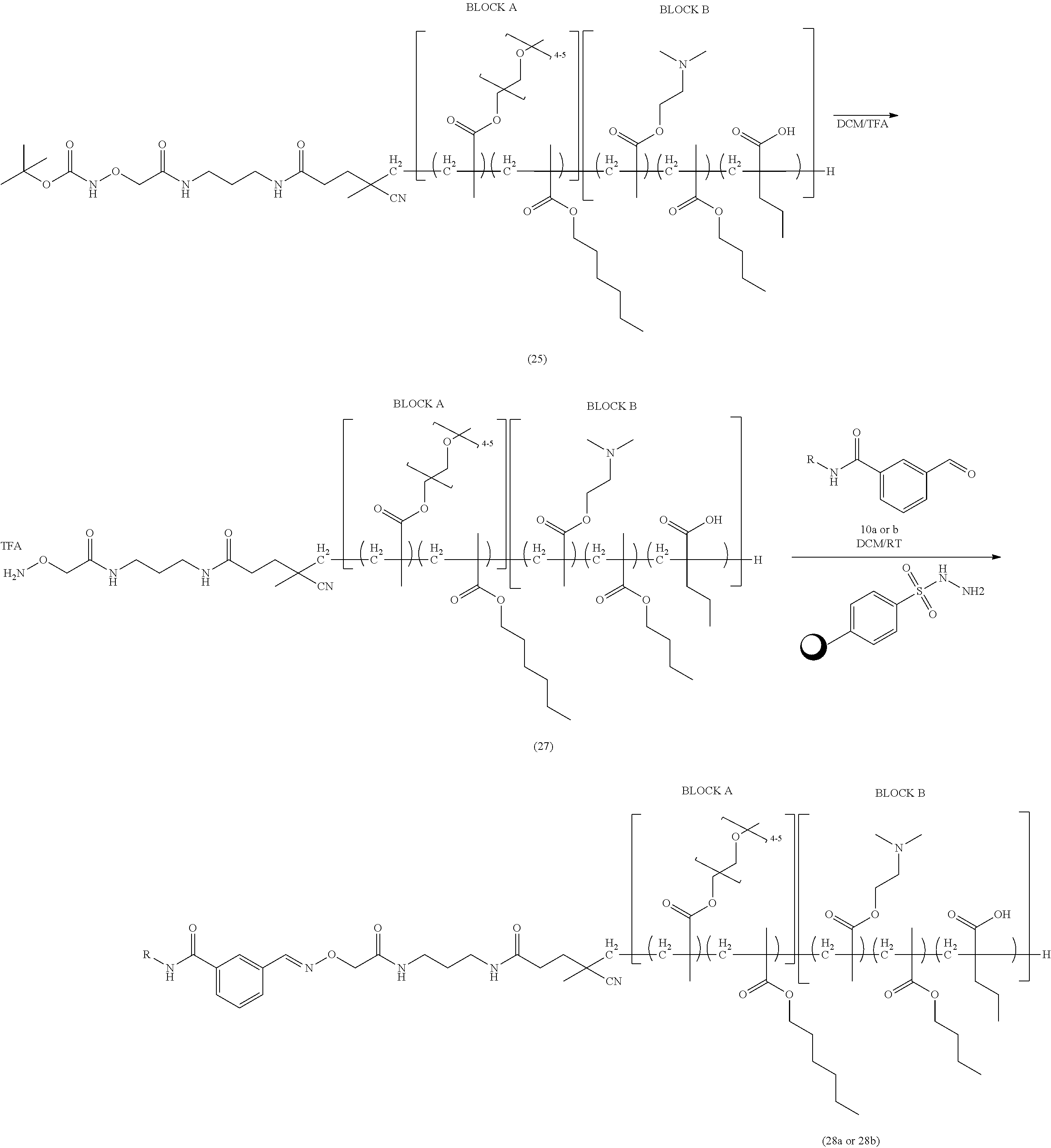

### a. Step 1. Removal of BOC from Endosomal Release Polymer 25

1.044 g of polymer was taken up in DCM (4 ml) and TFA (3.3 ml) was added. The reaction was stirred at RT for 2 hr. The reaction was concentrated in-vacuo for 60 mins. The residual TFA salt was used in the subsequent conjugation step without additional processing.

### b. Step 2. Oxime Conjugation

The residual material from the previous step was taken up in DCM (5 mL), 0.55 stoichiometric equivalents of the appropriate peptide benzaldehyde (18 a or b) added and the reaction stirred at RT for 72 hrs. An additional 0.55 stoichiometric equivalents of the appropriate peptide benzaldehyde was added and stirring continued for a further 72 hrs. Resin bound sequestration agent PS-TsNHNH2 (PS-tosylhydrazide) resin (3 equivalents) were added and the reaction left to stand for 24 hrs. The resin was removed by filtration washing with additional DCM. The solvent was removed in-vacuo drying to a constant weight. The residual material was taken up in acetone and dialysed against methanol for 2 days followed by water for 2 days. Lyophilising give the peptide oxime conjugated endosomal release polymers 28 a or b.

### Example 4: Ligands and Chemistry Used to Form siRNA-Ligand Conjugates

The following ligands and corresponding siRNA conjugates were synthesized to assess in vitro (Example 10) and in vivo activity (Example 11) in conjunction with a targeting endosome release polymer (Example 3). Peptide ligands were first synthesized using standard solid phase peptide synthesis protocols, then functionalized with an appropriate conjugation motif (e.g maleimide or DBCO) to facilitate coupling to the 3′ end of the sense strand of an siRNA molecule. The phosphate at the 3′ end of the siRNA was also accordingly modified for coupling to the ligand (e.g 3′ C6 amino or 3′ C6 sulfhydryl).

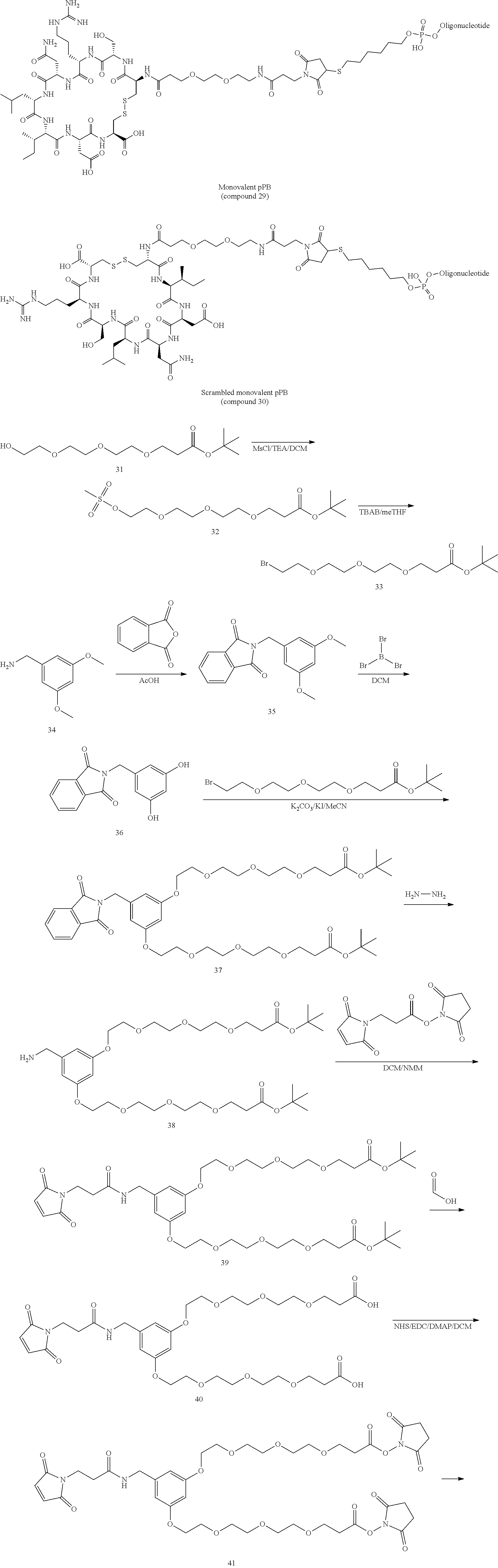

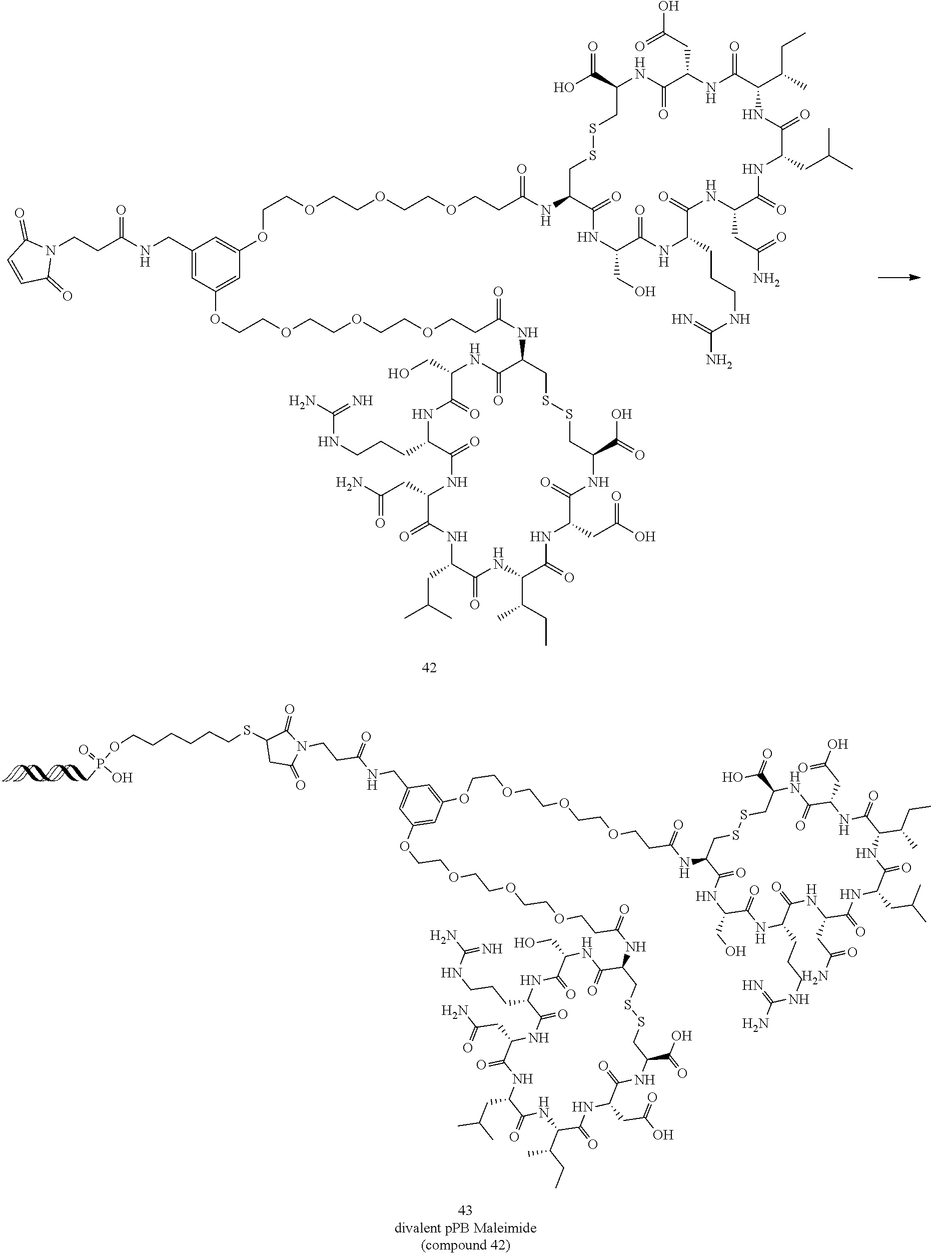

### a. Step 1: Synthesis of tert-butyl 3-(2-(2-(2-((methylsulfonyl)oxy)ethoxy)ethoxy)-ethoxy)propanoate

tert-Butyl-3-{2-[2-(2-hydroxyethoxy)ethoxy]ethoxy}propanoate (25 g, 90 mmol) and TEA (16.0 mL, 113 mmol were stirred in DCM at 0 °C. Methanesulfonyl chloride (11.3 g, 99 mmol) in DCM was added dropwise and the reaction stirred at RT for 1.5 hr. The organics were washed with saturated NaHCO₃, water and brine, dried (MgSO₄) and concentrated in-vacuo to give tert-butyl 3-(2-{2-[2-(methanesulfonyloxy)ethoxy]ethoxy}ethoxy)propanoate (32.1 g, Quant) which was used without purification.

### b. Step 2: Synthesis of tert-butyl 3-(2-(2-(2-bromoethoxy)ethoxy)ethoxy)propanoate

tert-Butyl 3-(2-{2-[2-(methanesulfonyloxy)ethoxy]ethoxy}ethoxy)propanoate (33 g, 92 mmol) and TBAB (39 g, 120 mmol) were heated at reflux for 2 hr. The reaction was cooled, washed with water and the aqueous back extracted with hexane. The combined organics were washed with brine, dried (Na₂SO₄), concentrated in-vacuo and the residue purified by automated flash chromatography (35% EtOAc/hex) to give tert-butyl 3-{2-[2-(2-bromoethoxy)ethoxy]ethoxy}propanoate (27.4 g, 87.1%)

### c. Step 3: Synthesis of 2-(3,5-dimethoxybenzyl)isoindoline-1,3-dione

(3,5-Dimethoxyphenyl)methanamine and isobenzofuran-1,3-dione were heated in AcOH at reflux for 16 h. The reaction was concentrated in-vacuo, the residue taken up in DCM, washed with 1M HCl, NaHCO₃, water and brine, dried (Na₂SO₄) and concentrated in-vacuo. The residue was purified by automated flash chromatography (35% EtOAc/Hex) to give 2-[(3,5-dimethoxyphenyl)methyl]isoindole-1,3-dione (9.03 g, 45.6%).

### d. Step 4: Synthesis of 2-(3,5-dihydroxybenzyl)isoindoline-1,3-dione

2-[(3,5-Dimethoxyphenyl)methyl]isoindole-1,3-dione (9 g, 30 mmol) was stirred in DCM at 0 °C. BBr₃ (11.7 mL, 121 mmol) was added dropwise and the reaction stirred for 16 hr allowing to warm to RT. The reaction was cooled to 0 °C, quenched with MeOH and concentrated in-vacuo. The residue was taken up in EtOAc, washed sequentially with 1M HCl, saturated NaHCO₃, water and brine, dried (MgSO₄) and concentrated in-vacuo to give 2-[(3,5-dihydroxyphenyl)methyl]isoindole-1,3-dione (7.8 g 95.7%) which was used without further purification.

### e. Step 5: Synthesis of tert-butyl 3-{2-[2-(2-{3-[2-(2-{2-[3-(tert-butoxy)-3-oxopropoxy]-ethoxy}ethoxy)ethoxy]-5-[(1,3-dioxoisoindol-2-yl)methyl]phenoxy}ethoxy)ethoxy]ethoxy}-propanoate

2-[(3,5-dihydroxyphenyl)methyl]isoindole-1,3-dione (10 g, 37 mmol), tert-butyl 3-{2-[2-(2-bromoethoxy)ethoxy]ethoxy}propanoate (30.4 g, 89 mmol) and K₂CO₃ were heated at reflux in MeCN for 16 hr. The reaction was cooled to RT, filtered, washed with additional MeCN and concentrated in-vacuo. The residue was by automated flash chromatography (60% EtOAc/hex) to give tert-butyl 3-{2-[2-(2-{3-[2-(2-{2-[3-(tert-butoxy)-3-oxopropoxy]ethoxy}ethoxy)ethoxy]-5-[(1,3-dioxoisoindol-2-yl)methyl]phenoxy}ethoxy)ethoxy]ethoxy}propanoate (14 g, 47.7%)

### f. Step 6: Synthesis of tert-butyl 3-[2-(2-{2-[3-(aminomethyl)-5-[2-(2-{2-[3-(tert-butoxy)-3-oxopropoxy]ethoxy}ethoxy)ethoxy]phenoxy]ethoxy}ethoxy)ethoxy]propanoate

tert-Butyl 3-{2-[2-(2-{3-[2-(2-{2-[3-(tert-butoxy)-3-oxopropoxy]ethoxy}ethoxy)-ethoxy]-5-[(1,3-dioxo-3a,7a-dihydroisoindol-2-yl)methyl]phenoxy}ethoxy)ethoxy]-ethoxy}propanoate (14 g, 17.7 mmol) and hydrazine hydrate (4.3 ml, 88 mmol) were stirred in MeOH at 50 °C for 16 hr. The resultant precipitate was removed by filtration and the filtrate concentrated in-vacuo. The residue was purified by automated flash chromatography (10% MeOH/DCM) to give tert-butyl 3-[2-(2-{2-[3-(aminomethyl)-5-[2-(2-{2-[3-(tert-butoxy)-3-oxopropoxy]ethoxy}ethoxy)ethoxy]phenoxy]ethoxy}ethoxy)ethoxy]propanoate (7.71 g, 66.1%).

### g. Step 7: Synthesis of tert-butyl 3-{2-[2-(2-{3-[2-(2-{2-[3-(tert-butoxy)-3-oxopropoxy]ethoxy}ethoxy)ethoxy]-5-{[3-(2,5-dioxopyrrol-1-yl)propanamido]methyl}phenoxy}ethoxy)ethoxy]ethoxy}propanoate

tert-Butyl-3-[2-(2-{2-[3-(aminomethyl)-5-[2-(2-{2-[3-(tert-butoxy)-3-oxopropoxy]ethoxy}ethoxy)ethoxy]phenoxy]ethoxy}ethoxy)ethoxy]propanoate (7.71 g, 11.7 mmol), 2,5-dioxopyrrolidin-1-yl 3-(2,5-dioxopyrrol-1-yl)propanoate (3.1 g, 11.7 mmol) and NMM (1.41 ml, 12.9 mmol) were stirred in DCM at RT for 36 ht. The reaction was concentrated in-vacuo and the residue purified by automated flash chromatography (2.5% MeOH/DCM) to give tert-butyl 3-{2-[2-(2-{3-[2-(2-{2-[3-(tert-butoxy)-3-oxopropoxy]ethoxy}ethoxy)ethoxy]-5-{[3-(2,5-dioxopyrrol-1-yl)propanamido]methyl}phenoxy}ethoxy)ethoxy]ethoxy}propanoate (9.0 g, 95.1%)

### h. Step 8: Synthesis of 3-[2-(2-{2-[3-(2-{2-[2-(2-carboxyethoxy)ethoxy]ethoxy}ethoxy)-5-{[3-(2,5-dioxopyrrolidin-1-yl)propanamido]methyl}phenoxy]ethoxy}ethoxy)ethoxy]propanoic acid

tert-butyl 3-{2-[2-(2-{3-[2-(2-{2-[3-(tert-butoxy)-3-oxopropoxy]ethoxy}ethoxy)ethoxy]-5-{[3-(2,5-dioxopyrrolidin-1-yl)propanamido]methyl}phenoxy}ethoxy)ethoxy]-ethoxy}propanoate (9.0 g, 11.1 mmol) was stirred in formic acid at RT for 16 hr. The reaction was concentrated in-vacuo azeotroping with toluene twice and drying under high vacuum to give 3-[2-(2-{2-[3-(2-{2-[2-(2-carboxyethoxy)ethoxy]ethoxy}ethoxy)-5-{[3-(2,5-dioxopyrrolidin-1-yl)propanamido]methyl}phenoxy]ethoxy}ethoxy)ethoxy]propanoic acid quantitatively which was used with purification.

### i. Step 9: Synthesis of 2,5-dioxopyrrolidin-1-yl 3-(2-{2-[2-(3-{[3-(2,5-dioxopyrrol-1-yl)propanamido]methyl}-5-{2-[2-(2-{3-[(2,5-dioxopyrrolidin-1-yl)oxy]-3-oxopropoxy}ethoxy)ethoxy]ethoxy}phenoxy)ethoxy]ethoxy}ethoxy)propanoate

3-[2-(2-{2-[3-(2-{2-[2-(2-carboxyethoxy)ethoxy]ethoxy}ethoxy)-5-{[3-(2,5-dioxopyrrol-1-yl)propanamido]methyl}phenoxy]ethoxy}ethoxy)ethoxy]propanoic acid (8 g, 11.5 mmol), N-hydroxysuccinimde (3.6 g, 31. 4 mmol) and DMAP were stirred in DCM at RT for 5 hr. The reaction was concentrated in-vacuo and purified by automated flash chromatography (10% MeOH/DCM) to give 2,5-dioxopyrrolidin-1-yl 3-(2-{2-[2-(3-{[3-(2,5-dioxopyrrol-1-yl)propanamido]methyl}-5-{2-[2-(2-{3-[(2,5-dioxopyrrolidin-1-yl)oxy]-3-oxopropoxy}ethoxy)ethoxy]ethoxy}phenoxy)ethoxy]-ethoxy}ethoxy)propanoate (42) (3.7 g, 36.2%)

pPB conjugation and siRNA synthesis were conducted as described elsewhere to give aromatic dimeric pPB (compound 43) and aromatic dimeric scrambled pPB (compound 44)

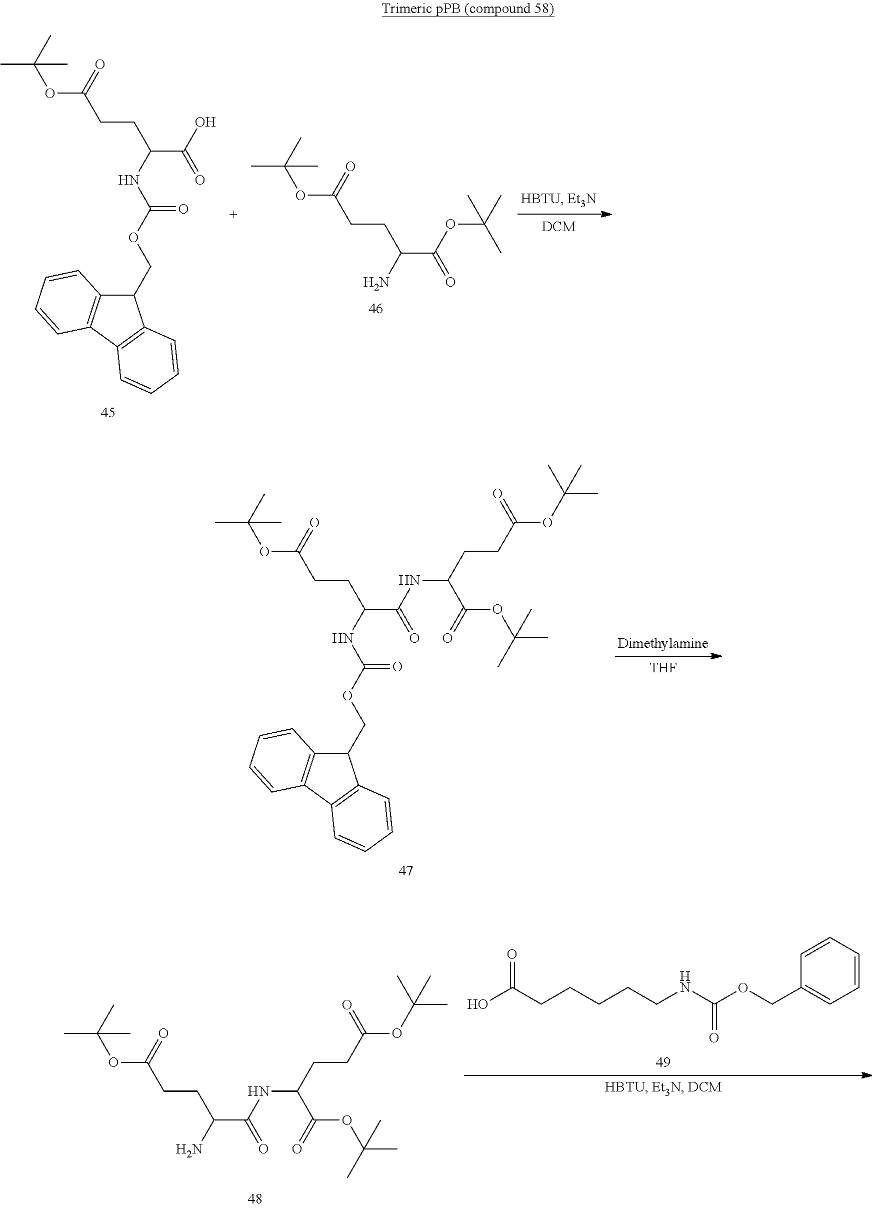

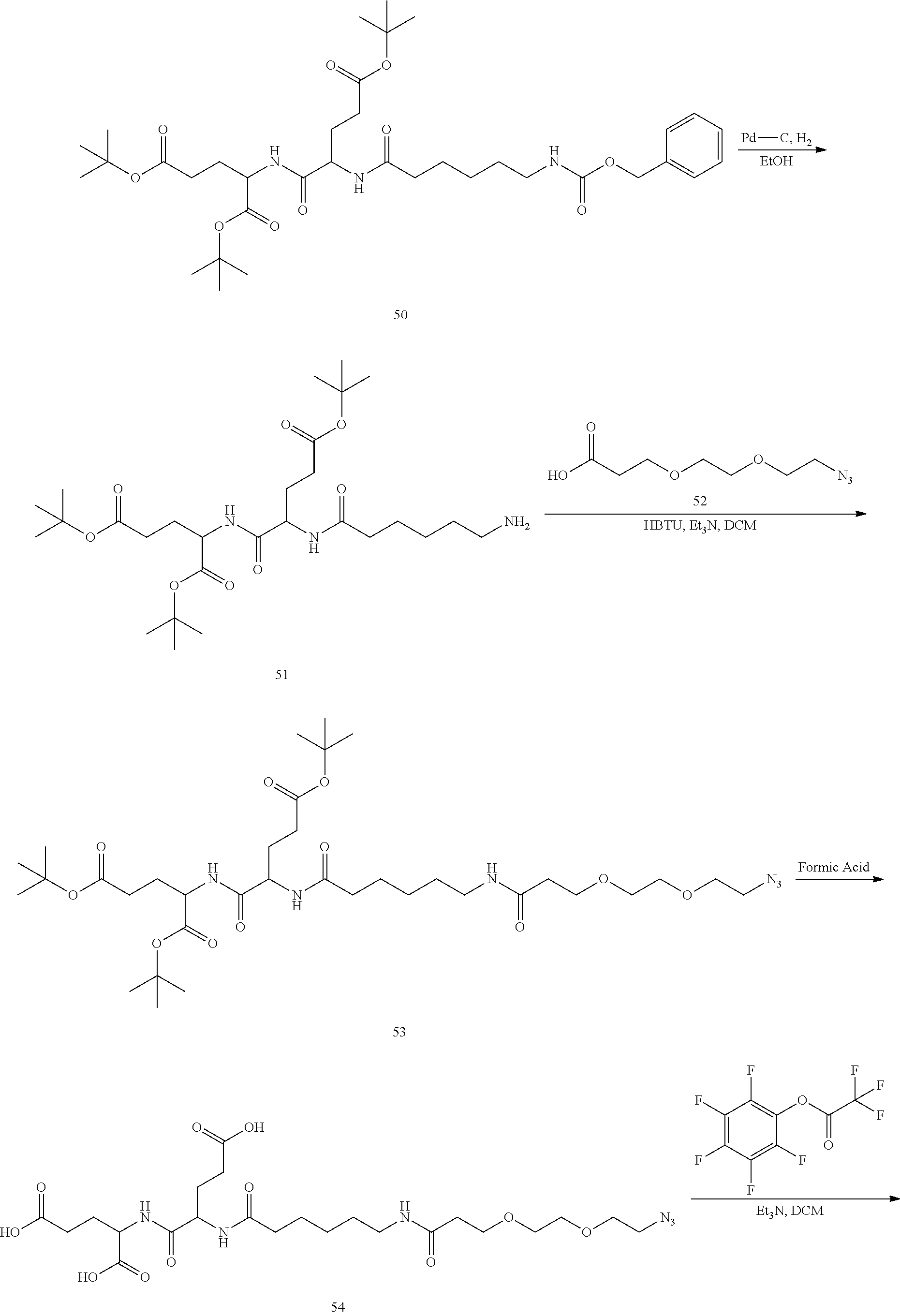

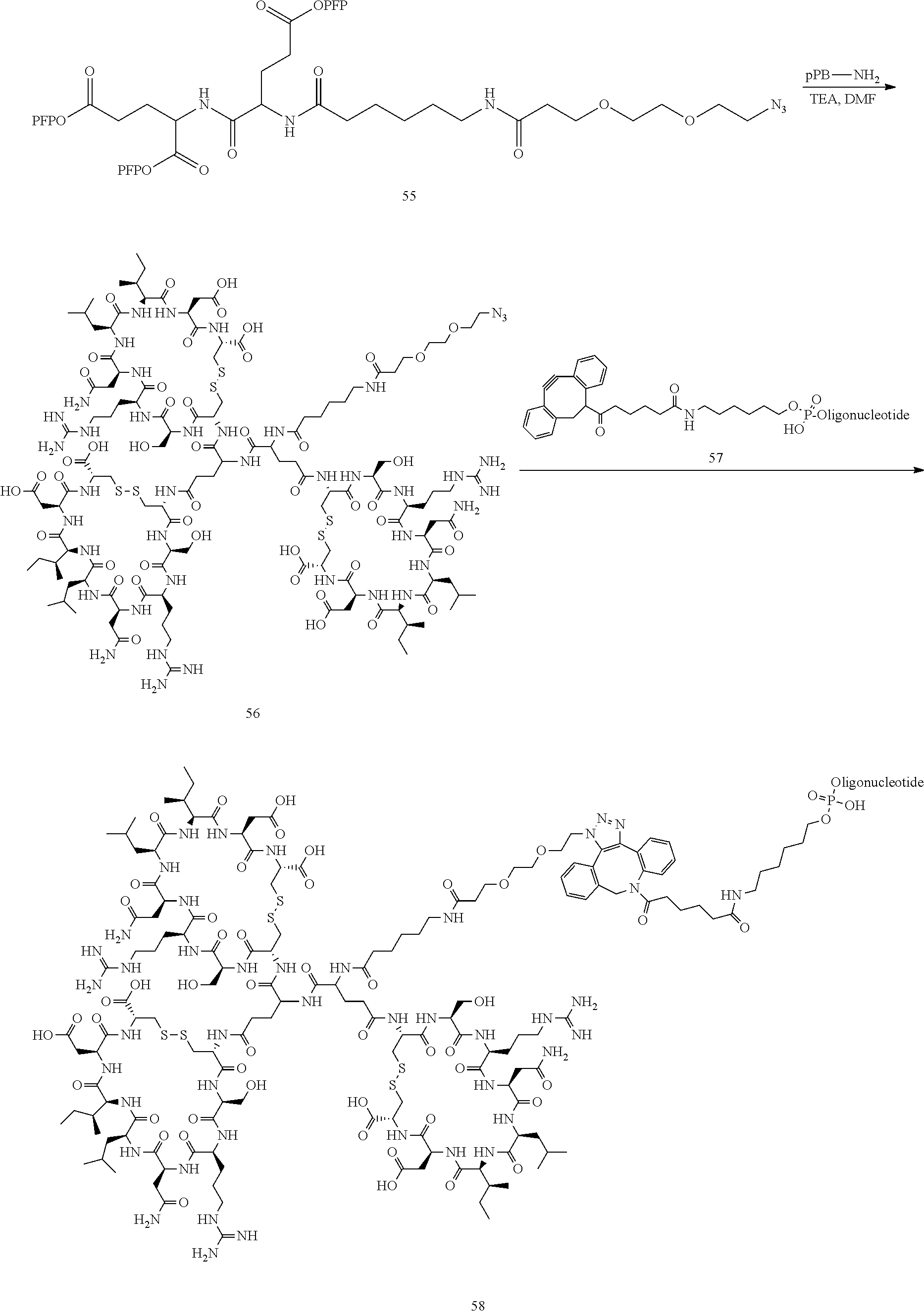

Trimeric pPB (compound 58) can be prepared according to the procedure described in the scheme above.

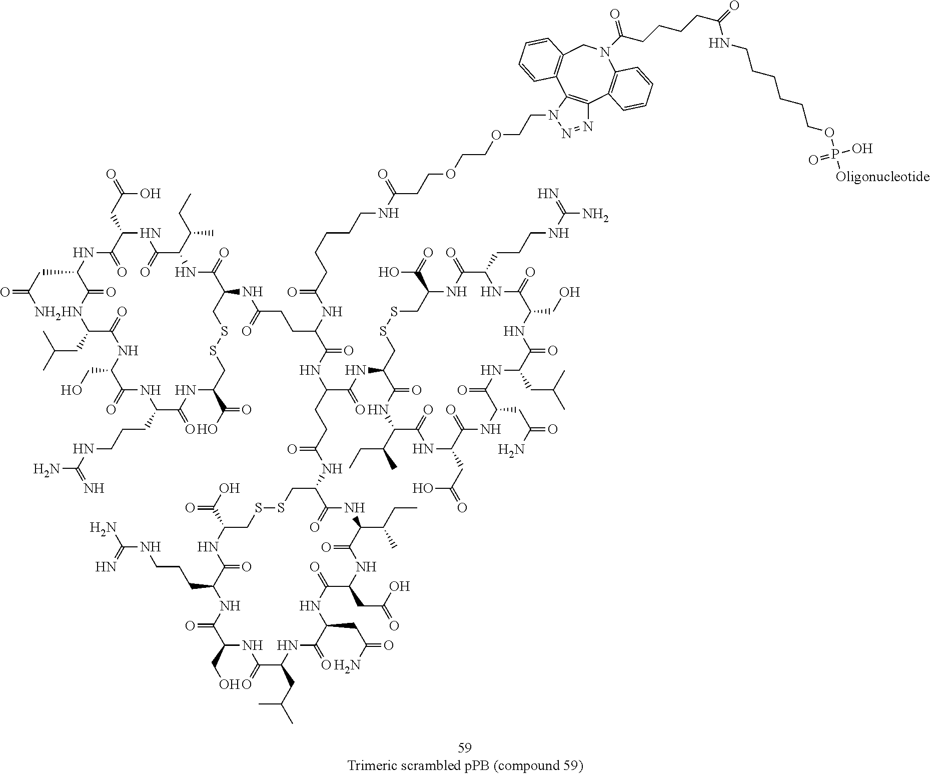

Trimeric scrambled pPB (compound 59) can be prepared using an analogous procedure to that described for compound 58.

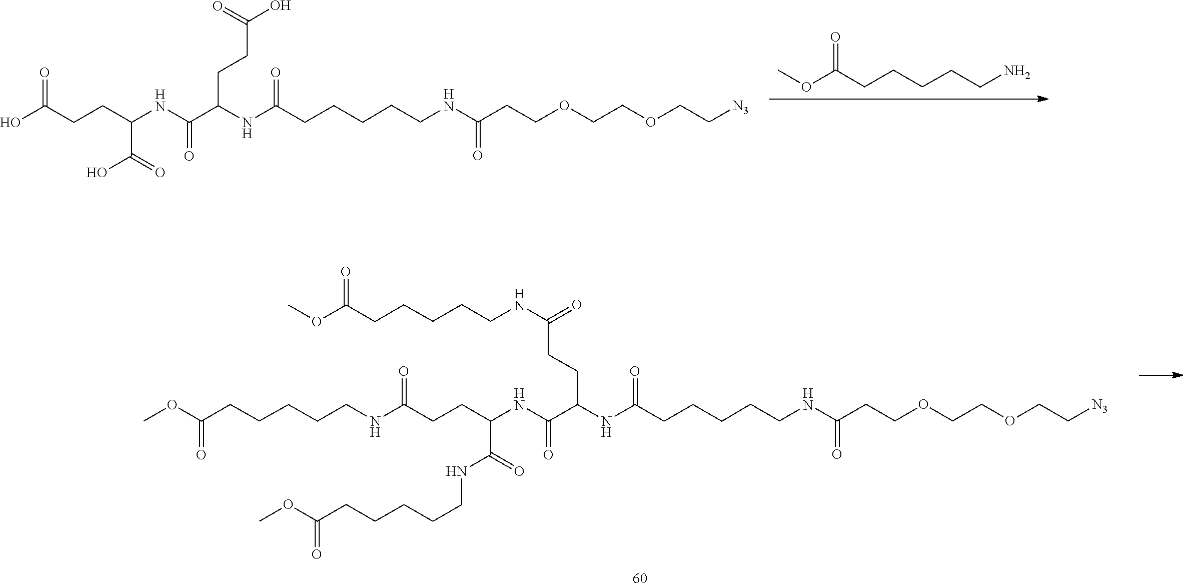

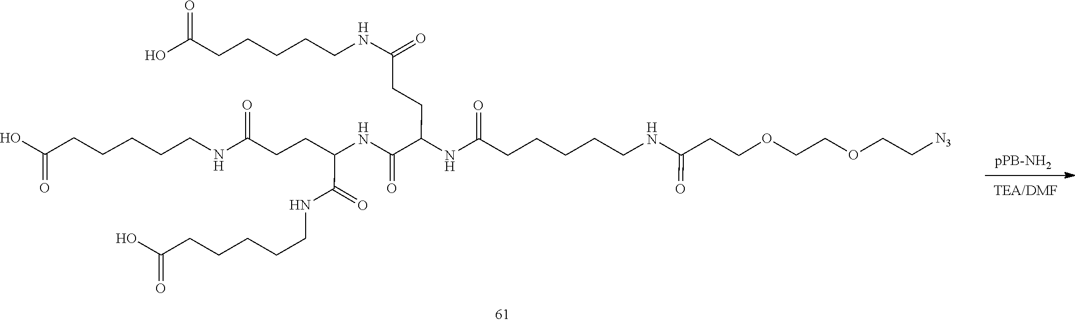 [SMILES: [N-]=[N+]=NCCOCCOCCC(=O)NCCCCCC(=O)NC(CCC(=O)NCCCCCC(=O)O)C(=O)NC(CCC(=O)NCCCCCC(=O)O)C(=O)NCCCCCC(=O)O]

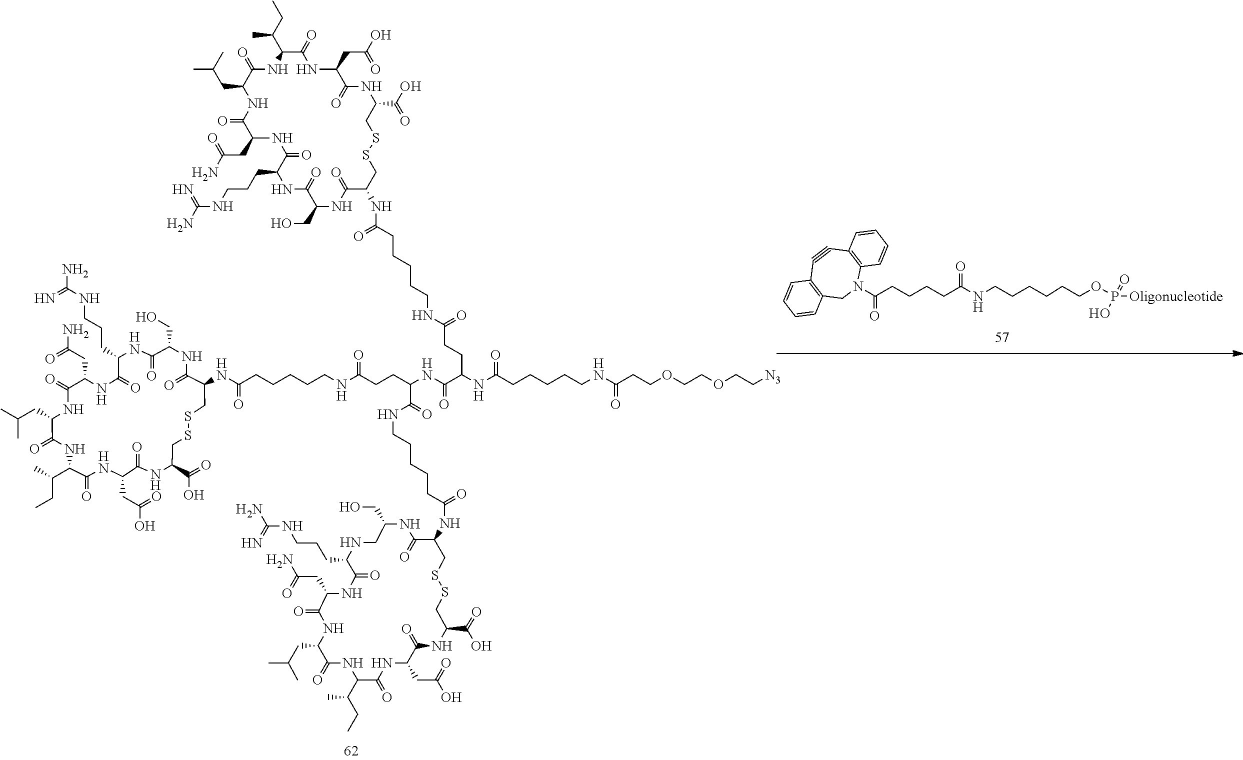

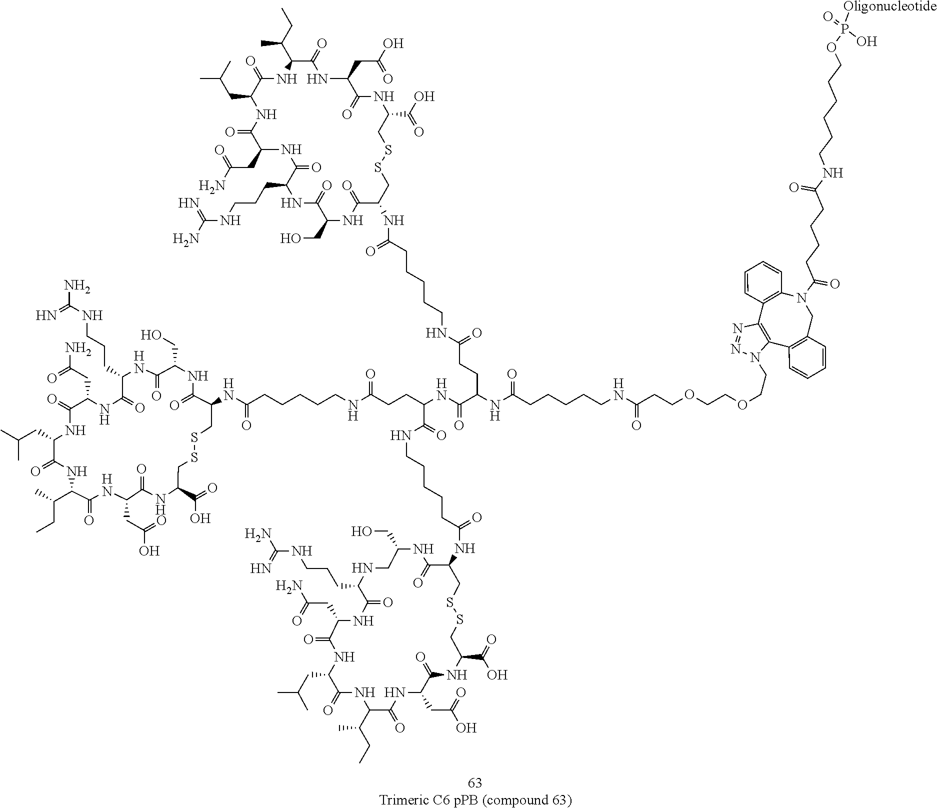

Trimeric C6 pPB (compound 63) can be prepared according to the procedure described in the scheme above.

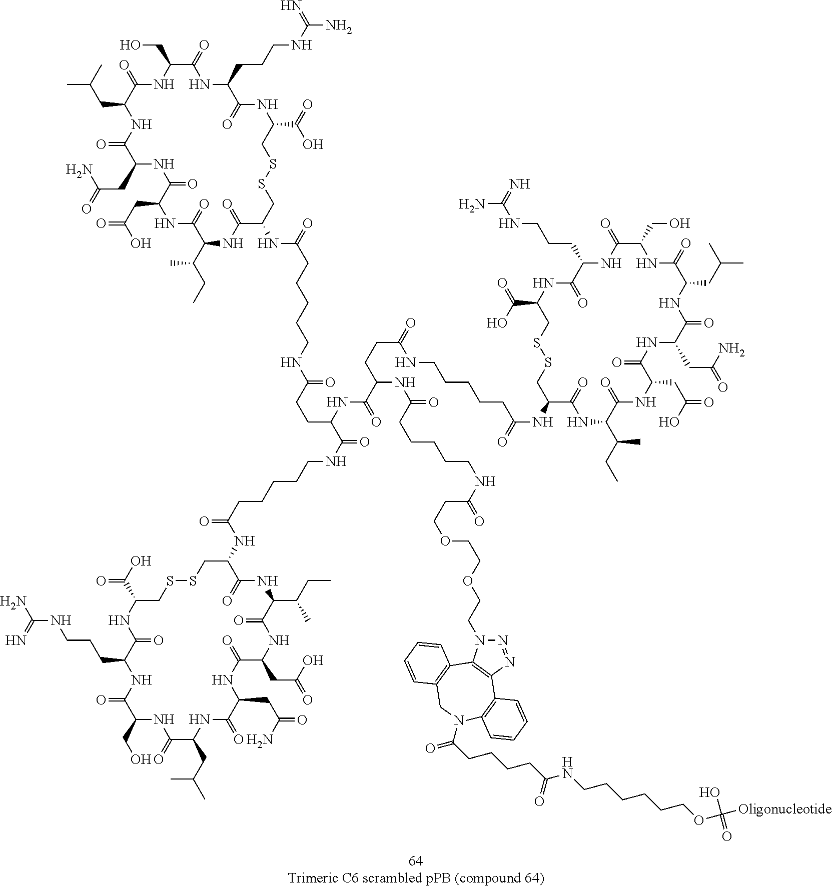

Trimeric C6 scrambled pPB (compound 64) can be prepared using an analogous procedure to that described for compound 63 pPB conjugation and siRNA synthesis were conducted as described elsewhere to give trimeric pPB (compound 58), trimeric scrambled pPB (compound 59), trimeric C6 pPB (compound 63) and trimeric C6 scrambled pPB (compound 64)

### Example 5: Ligand siRNA Conjugates

The following siRNA-ligand conjugates, as illustrated in Example 3, were prepared using automated oligonucleotide synthesis using standard procedures. Nucleotide deprotection and cleavage from the solid support, afforded the 3′ modified sense strand and the corresponding and complementary anti-sense strand. After purification by dual HPLC, the ligand conjugated siRNA duplex was formed by annealing. Table 1 lists siRNA sequences, ID's and mass analysis.

TABLE 1
siRNA conjugates

| | | | Mass Analysis | |
|---|---|---|---|---|
| | | Seq. | (MALDI; Da.) | |
| siRNA | Sequence (5′-3′) | ID | Calc. | Found |
| Monovalent | Sense strand: | SEQ ID | 7793.9 | 7794.38 |
| pPB siPPIB | mGsmGmAmAfAmGfAfCfUmGmUmUmCmCmAmAmAmAsmA | NO: 2 | | |
| (29a) | [29] | | | |
| | Antisense strand: | SEQ ID | 6836.53 | 6836.93 |
| | mUsfUsmUmUmUfGmGfAfAmCmAmGmUfCmUfUmUmCmCsmUs | NO: 3 | | |
| | mU | | | |
| Scrambled | Sense strand: | SEQ ID | 7793.9 | 7793.8 |
| monovalent | mGsmGmAmAfAmGfAfCfUmGmUmUmCmCmAmAmAmAsmA | NO: 4 | | |
| pPB siPPIB | [30] | | | |
| (30a) | Antisense strand: | SEQ ID | 6836.53 | 6836.93 |
| | mUsfUsmUmUmUfGmGfAfAmCmAmGmUfCmUfUmUmCmCsmUs | NO: 5 | | |
| | mU | | | |
| aromatic | Sense strand: | SEQ ID | 9067.34 | 9067.67 |
| divalent pPB | mGsmGmAmAfAmGfAfCfUmGmUmUmCmCmAmAmAmAsmA | NO: 6 | | |
| siPPIB (43a) | [43] | | | |
| | Antisense strand: | SEQ ID | 6835.53 | 6835.3 |
| | mUsfUsmUmUmUfGmGfAfAmCmAmGmUfCmUfUmUmCmCsmUs | NO: 7 | | |
| | mU | | | |
| aromatic | Sense strand: | SEQ ID | 9067.34 | 9068.19 |
| divalent | mGsmGmAmAfAmGfAfCfUmGmUmUmCmCmAmAmAmAsmA | NO: 8 | | |
| scrambled | [44] | | | |
| pPB siPPIB | Antisense strand: | SEQ ID | 6836.53 | 6835.3 |
| (44a) | mUsfUsmUmUmUfGmGfAfAmCmAmGmUfCmUfUmUmCmCsmUs | NO: 9 | | |
| | mU | | | |
| Trimeric | Sense strand: | SEQ ID | 10144.57 | 10145.00 |
| pPB siPPIB | mGsmGmAmAfAmGfAfCfUmGmUmUmCmCmAmAmAmAsmA | NO: 10 | | |
| (58a) | [58] | | | |
| | Antisense strand: | SEQ ID | 6836.53 | 6835.3 |
| | mUsfUsmUmUmUfGmGfAfAmCmAmGmUfCmUfUmUmCmCsmUs | NO: 11 | | |
| | mU | | | |
| Trimeric | Sense strand: | SEQ ID | 10144.57 | 10145.11 |
| scrambled | mGsmGmAmAfAmGfAfCfUmGmUmUmCmCmAmAmAmAsmA | NO: 12 | | |
| pPB siPPIB | [59] | | | |
| (59a) | Antisense strand: | SEQ ID | 6836.53 | 6837.1 |
| | mUsfUsmUmUmUfGmGfAfAmCmAmGmUfCmUfUmUmCmCsmUs | NO: 13 | | |
| | mU | | | |
| Trimeric C6 | Sense strand: | SEQ ID | 10484.04 | 10484.49 |
| pPB siPPIB | mGsmGmAmAfAmGfAfCfUmGmUmUmCmCmAmAmAmAsmA | NO: 14 | | |
| (63a) | [63] | | | |
| | Antisense strand: | SEQ ID | 6836.53 | 5835.3 |
| | mUsfUsmUmUmUfGmGfAfAmCmAmGmUfCmUfUmUmCmCsmUs | NO: 15 | | |
| | mU | | | |
| Trimeric C6 | Sense strand: | SEQ ID | 10484.04 | 19484.35 |
| scrambled | mGsmGmAmAfAmGfAfCfUmGmUmUmCmCmAmAmAmAsmA | NO: 16 | | |
| pPB siPPIB | [64] | | | |
| (64a) | Antisense strand: | SEQ ID | 6836.53 | 6835.3 |
| | mUsfUsmUmUmUfGmGfAfAmCmAmGmUfCmUfUmUmCmCsmUs | NO: 17 | | |
| | mU | | | |
| Trimeric | Sense Strand: | SEQ ID | 10130.49 | 10130.32 |
| pPB siHsp47 | mGsmAmGmAfCmAfCfAfUmGmGmGmUmGmCmUmAmUsmU | NO: 18 | | |
| (58b) | [58] | | | |
| | Antisense Strand: | SEQ ID | 6942.71 | 6941.69 |
| | mAsfAsmUmAmGfCmAfCfCmCmAmUmGfUmGfUmCmUmCsmAs | NO: 19 | | |
| | mG | | | |
| Trimeric | Sense strand: | SEQ ID | 10069.4 | 10069.72 |
| pPB siLuc2 | mGsmAmUmUfAmUfGfUfCmCmGmGmUmUmAmUmGmUsmA | NO: 20 | | |
| (58c) | (58) | | | |
| | Antisense: | SEQ ID | 6934.74 | 6936.28 |
| | mUsfAsmCmAmUfAmAfCfCmGmGmAmCfAmUfAmAmUmCsmAs | NO: 21 | | |
| | mU | | | |

A = adenine; G = guanine; U = uracil; C = cytosine; mN = 2′OMe ribose modification; fN = 2′fluoro ribose modification; s = phosphorothioate (PS) modification.

### Example 6. Uptake of Monovalent and Trivalent pPB Ligand by HSC-T6 and NIH13T3 Cells In Vitro

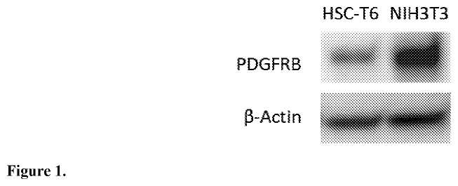

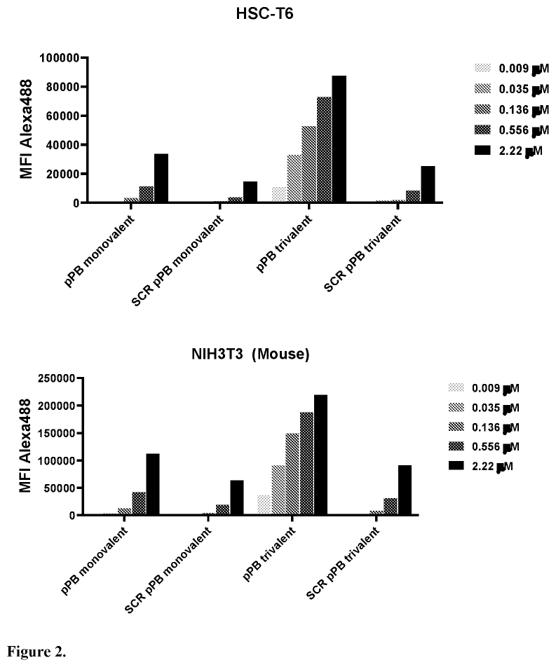

HSC-T6 is an immortalized rat hepatic stellate cell line and NIH3T3 is a murine embryonic fibroblast cell line. Both cell lines express the pPB target receptor PDGFRB with NIH3T3 cells exhibiting higher expression (FIG. 1).

### Preparation of Biotin-pPB/AF488-Streptavidin Complex

Biotinylated monovalent or trivalent pPB ligand were reconstituted in DMSO and functionalized by incubating with Alexa Fluor 488-labelled streptavidin in Tyrode buffer (containing 10 mM HEPES, 5.6 mM glucose, 10 mM KCl, 35 mM NaCl, 0.4 mM MgCl, 1.0 mM CaCl2 and 0.1% BSA, pH 7.3) at 4-C overnight at a molar ratio of 4.5:1 of biotinylated ligands to biotin binding sites.

### Cell Culture

HSC-T6 cells were cultured in rat HSC medium (DMEM-High Glucose without Sodium Pyrvate containing 10% FBS and 2.5 mM L-Glutamine). NIH3T3 cells were cultured in MEM containing 10% FBS and 2.5 mM L-Glutamine.

### Western Blot Analysis of PDGFRB Expression

Cultured HSC-T6 cells and NIH3T3 cells were lysed with RIPA buffer. Cell lysates were cleared by centrifugation at 13000×g for 15 min and separated with SDS PAGE, transferred to PVDF membrane. and probed using anti-PDGFRB (cell signaling) and anti-beta-actin antibodies (Cell Signaling) in 5% BSA Tris-buffered saline and Tween-20 buffer.

### Uptake of Biotin-pPB/AF488-Streptavidin Complex

HSC-T6 or NIH3T3 cells were seeded into sterile 96 wells plates (20000/well) and cultured for 48 h at 37 °C. The previous prepared biotin-pPB/AF488-streptavidin complex was diluted to 2.22, 0.556, 0.136, 0.035 and 0.009 μM (based on streptavidin molar concentration) in Tyrode buffer and was kept on ice. Prior to complex incubation, the cells were washed three times with ice cold Tyrode buffer and functionalized streptavidin was added to each well which was incubated at 4 °C for 1.5 h. After incubation, the cells were washed three times with ice-cold Tyrode buffer to remove any unbound ligand complex, before adding 37 °C preheated complete media and immediately incubating the cells in 37 °C for 1 h to allow endocytosis of receptor bound ligand-complexes. After incubation, the cells were washed once with DPBS and 50 μL of 0.25% Trypsin-EDTA was added to each well. After incubation at 37 °C for 3-4 min, 100 μL of media was added to each well to inactivate the trypsin, and detachment of cells was accomplished with vigorous mixing. The cells were transferred to a V-bottom 96-well plate, spun at 1200 RPM for 5 min, and washed twice with DPBS before resuspension in DPBS containing Live/Dead Red stain. After 30 min at 4 °C, the cells were centrifuged, and the pellet resuspended in stain buffer prior to flow cytometry analysis.

### Flow Cytometry Analysis

Analyses were performed on a FACS-Canto II using the software FACS Diva. As a marker for viability, cells were stained with Live/Dead Red. The forward scatter and side scatter gate were set to include all viable cells.

Approximately 10000-15000 cells were counted for each sample and binding/uptake was determined as increased intensity in green fluorescence at 488 nm detected in the FL1 channel. The mean fluorescence intensity (MFI) of cells incubated with functionalized streptavidin minus the MFI of cells incubated without functionalized streptavidin (free fluorophore only) was used to determine binding/uptake.

### Results

Uptake of monovalent and trivalent pPB ligand complexes was observed in both HSC-T6 cells and NIH3T3 cells as evidenced by the dose dependent increase of AF488 fluorescence in the treated cells (FIG. 2). Trivalent pPB ligands showed ~60-fold higher binding affinity than monovalent pPB ligands. Ligand complex with scrambled monovalent pPB peptide showed ~4-fold lower potency than monovalent pPB ligand complex. This potency difference increased to ~60 fold in the trivalent ligands. The mean fluorescence intensity (MFI) from the ligand was higher in NIH3T3 cells than HSC-T6 cells, correlated to the PDGFRB receptor expression level in these two cell lines. These results demonstrated the effective uptake of pPB ligands in PDGFRB expressing cells.

### Example 7. Uptake of Divalent and Trivalent pPB Ligands by Primary Human Hepatic Stellate Cells In Vitro

### Materials and Methods

### Preparation of Biotin-pPB/AF488-Streptavidin Complex

Biotinylated divalent, trivalent pPB or scrambled trivalent pPB ligands were reconstituted in DMSO and functionalized by incubating with Alexa Fluor 488-labelled streptavidin in Tyrode buffer (containing 10 mM HEPES, 5.6 mM glucose, 10 mM KCl, 35 mM NaCl, 0.4 mM MgCl, 1.0 mM CaCl2) and 0.1% BSA, pH 7.3) at 4-C overnight at a molar ratio of 4.5:1 of biotinylated ligands to biotin binding sites.

### Cell Culture

pHHSC were cultured using a human hepatic stellate cell culture kit from ScienCell.

### Uptake of Biotin-pPB/AF488-Streptavidin Complex

pHHSCs were seeded into sterile 96 wells plates (6000 cells/well) and cultured for 48 h at 37 °C. The previous prepared biotin-pPB/AF488-streptavidin complex was diluted to 3, 1, 0.3 and 0.1 μM (based on streptavidin molar concentration) in Tyrode buffer and was kept on ice. Prior to complex incubation, the cells were washed three times with ice cold Tyrode buffer and functionalized streptavidin was added to each well which was incubated at 4 °C for 1.5 h. After incubation, the cells were washed three times with ice-cold Tyrode buffer to remove any unbound ligand complex, before adding 37 °C preheated complete media and immediately incubating the cells in 37 °C for 1 h to allow endocytosis of receptor bound ligand-complexes. After incubation, the cells were washed once with DPBS and 50 μL of 0.025% Trypsin-EDTA was added to each well. After incubation at 37 °C for 3-4 min, 100 μL of Trypsin Neutralization Solution (TNS) was added to each well to inactivate the trypsin, and detachment of cells was accomplished with vigorous mixing. The cells were transferred to a V-bottom 96-well plate, spun at 1200 RPM for 5 min, and washed twice with stain buffer before resuspension in stain buffer containing DAPI (4′,6-diamidino-2-phenylindole) prior to flow cytometry analysis.

### Flow Cytometry Analysis

Analyses were performed on a FACS-Canto II using the software FACS Diva. As a marker for viability, cells were stained with DAPI. The forward scatter and side scatter gate were set to include all viable cells. Approximately 10000-15000 cells were counted for each sample and binding/uptake was determined as increased intensity in green fluorescence at 488 nm detected in the FL1 channel. The mean fluorescence intensity (MFI) of cells incubated with functionalized streptavidin minus the MFI of cells incubated without functionalized streptavidin (free fluorophore only) was used to determine binding/uptake.

### Results

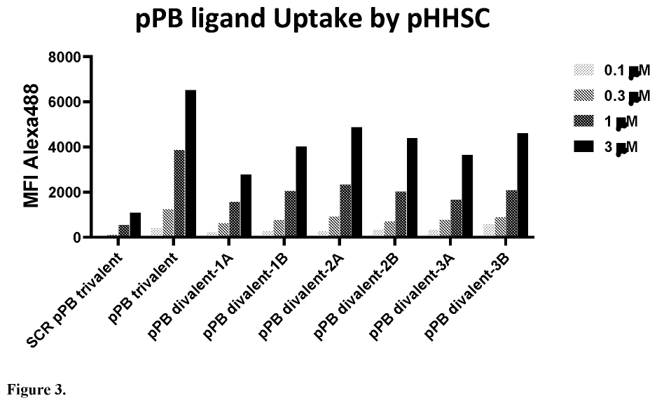

Uptake of divalent and trivalent pPB ligand complexes was observed in pHHSCs as evidenced by the dose dependent increase of AF488 fluorescence in the treated cells (FIG. 3). Divalent pPB ligands showed slightly lower binding activity than trivalent pPB ligands but were much more potent than the scrambled trivalent pPB ligand. These results demonstrates that pPB ligand valency affects binding affinity.

### Example 8. Uptake of Trivalent pPB Ligand by NIH3T3, LX-2 and Primary Human HSC In Vitro

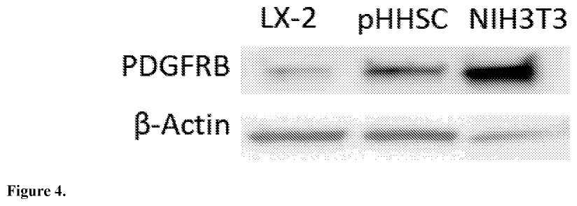

LX-2 is an immortalized human hepatic stellate cell line and NIH3T3 is a murine embryonic fibroblast cell line. All 3 cell lines express the pPB target receptor PDGFRB with NIH3T3 cells exhibited highest expression (FIG. 4).

### Materials and Methods

### Preparation of Biotin-pPB/AF488-Streptavidin Complex

Biotinylated trivalent pPB ligand was reconstituted in DMSO and functionalized by incubating with Alex Fluor 488-labelled streptavidin in Tyrode buffer (containing 10 mM HEPES, 5.6 mM glucose, 10 mM KCl, 35 mM NaCl, 0.4 mM MgCl, 1.0 mM CaCl2) and 0.1% BSA, pH 7.3) at 4-C overnight at a molar ratio of 4.5:1 of biotinylated ligands to biotin binding sites.

### Cell Culture

LX-2 cells were cultured in DMEM-High Glucose containing 10% FBS and 2.5 mM L-Glutamine. NIH3T3 cells were cultured in MEM with containing 10% FBS and 2.5 mM L-Glutamine. pHHSC were cultured using the human hepatic stellate cell culture kit from ScienCell.

### Western Blot Analysis of PDGFRB Expression

Cultured LX-2, NIH3T3 and pHHSC cells were lysed with RIPA buffer. Cell lysates were cleared by centrifugation at 13000×g for 15 min and separated with SDS PAGE, transferred to PVDF membrane. and probed using anti-PDGFRB (cell signaling) and anti-beta-actin antibodies (Cell Signaling) in 5% BSA Tris-buffered saline and Tween-20 buffer.

### Uptake of Biotin-pPB/AF488-Streptavidin Complex

LX-2, NIH3T3 or pHHSC cells were seeded into sterile 96 wells plates (20000/well) and cultured for 48 h at 37 °C. The previous prepared biotin-pPB/AF488-streptavidin complex was diluted to 3, 1, 0.3, and 0.1 μM (based on streptavidin molar concentration) in Tyrode buffer and was kept on ice. Prior to complex incubation, the cells were washed three times with ice cold Tyrode buffer and functionalized streptavidin was added to each well which was incubated at 4 °C for 1.5 h. After incubation, the cells were washed three times with ice-cold Tyrode buffer to remove any unbound ligand complex, before adding 37 °C preheated complete media and immediately incubating the cells in 37 °C for 1 h to allow endocytosis of receptor bound ligand-complexes. After incubation, the cells were washed once with DPBS and 50 μL of 0.25% Trypsin-EDTA was added to each well. After incubation at 37 °C for 3-4 min, 100 μL of media was added to each well to inactivate the trypsin, and detachment of cells was accomplished with vigorous mixing. The cells were transferred to a V-bottom 96-well plate, spun at 1200 RPM for 5 min, and washed twice with stain buffer before resuspension in stain buffer containing DAPI prior to flow cytometry analysis.

### Flow Cytometry Analysis

Analyses were performed on a FACS-Canto II using the software FACS Diva. As a marker for viability, cells were stained with DAPI. The forward scatter and side scatter gate were set to include all viable cells. Approximately 10000-15000 cells were counted for each sample and binding/uptake was determined as increased intensity in green fluorescence at 488 nm detected in the FL1 channel. The mean fluorescence intensity (MFI) of cells incubated with functionalized streptavidin minus the MFI of cells incubated without functionalized streptavidin (free fluorophore only) was used to determine binding/uptake.

### Results

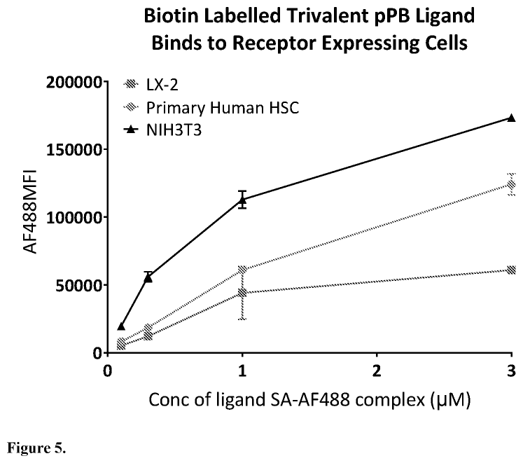

Uptake of trivalent pPB ligand complexes was observed in HSC-T6, LX-2 and pHHSC as evidenced by the dose dependent increase of fluorescence intensity in the treated cells (FIG. 5). The mean fluorescence intensity (MFI) in the 3 cell types correlated with the target receptor (PDGFBR) expression levels. These results demonstrate effective uptake of pPB ligands in PDGFRB expressing cells including human hepatic stellate cells.

### Example 9. Trivalent pPB Conjugate siRNA Enabled Gene Silencing in NIH3T3 Cells in Vitro when Treated Together with Chloroquine

PPIB is a widely expressed protein coding gene and was used as a surrogate siRNA target in this study. NIH3T3 cells express high levels of PDGFRB and exhibited the best pPB ligand uptake efficiency in the previous experiments. To address limited endosome release in siRNA conjugate delivery, we stimulated the cells with chloroquine after siRNA conjugate treatment.

### Materials and Methods

### Cell Culture

NIH3T3 cells were cultured in MEM with containing 10% FBS and 2.5 mM L-Glutamine.

Treatment of siRNAs in NIH3T3 Cells

NIH3T3 cells were seeded into sterile 96 wells plates (10000/well) and cultured for 24 h at 37 °C. Cells were then washed with 100 μL OptiMEM and then incubated with 0.4, 0.1 or 0.025 μM of TripPB-siPPIB, Tri-Scr-pPB-siPPIB, unconjugated siPPIB or siLuc2 in OptiMEM for 4 h. The siRNAs or conjugates were then removed, and the cells were treated with 50 μM of chloroquine in complete culture medium for another 20 h. Culture medium were removed after treatment completion and the cells were lysed with QuantiGene Lysis Mixture for next step gene expression quantification.

### QuantiGene Branched DNA Assay

To evaluate gene silencing activity, cell lysates collected after siRNA treatment were subject to QuantiGene branched DNA assay according to the manufacturer's protocol. In brief, cell lysates were incubated with capture probes targeting mPpib (target gene) and mGapdh (endogenous control) at 55 °C for 18-20 h. After washing, the plates were incubated with pre-amplification probes and amplification probes to amplify the signal. The excess probes were washed off and assay substrates added to allow quantifying luminescence using a plate reader. Ppib signal was normalized to the signal from Gapdh.

### Results

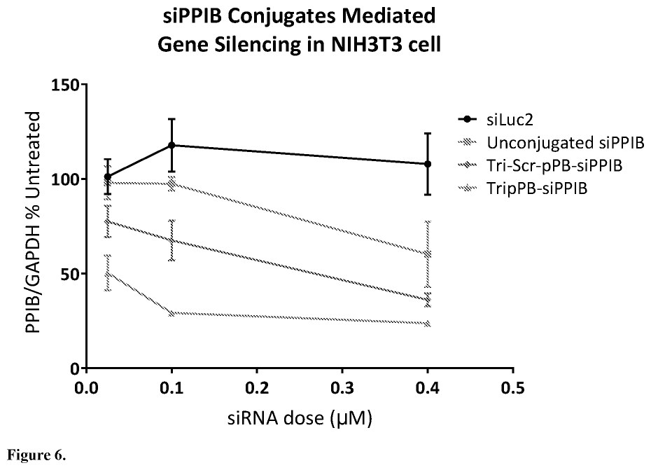

Dose dependent target gene (Ppib) silencing was observed in TripPB-siPPIB, Tri-Scr-pPB-siPPIB and unconjugated PPIB treated cells but not in the siLuc2 treated cells, demonstrating targeted gene silencing mediated by siPPIB (FIG. 6). Importantly, TripPB conjugated siRNA conferred the highest activity as compared to Tri-Scr-pPB-siPPIB and unconjugated PPIB, suggesting that TripPB ligand enables more efficient delivery of siRNA. Consistent with the ligand binding results, Tri-Scr-pPB ligand conjugation moderately improved RNAi activity as compared to unconjugated siRNA. Unconjugated siRNA was only active at the highest tested dose (0.40 μM), potentially through a passive uptake delivery mechanism. These results demonstrated that trivalent pPB ligand conjugation could enhance delivery of siRNA to NIH3T3 cells.

### Example 10. Trivalent pPB Conjugate siRNA with pPB Functionalized Endosome Release Polymer Enabled Gene Silencing in pHHSC Cells in Vitro

### Materials and Methods

The polymer was dissolved in 10 mM phosphate/200 mM sucrose PBS (pH 7), up to 40 mg/mL followed by successive filtration (3-4 times) through a 0.2-micron sterile filter.

### Cell Culture

pHHSC were cultured using the human hepatic stellate cell culture kit from ScienCell.

Treatment of siRNAs in pHHSC Cells

pHHSC cells were seeded into sterile 96 wells plates (5000/well) and cultured for 24 h at 37 °C. Cells were then washed with 100 μL OptiMEM and then treated with 0.3 or 0.1 μM of TripPB-siPPIB or unconjugated siPPIB in OptiMEM for 4 h. GalNAc or pPB functionalized ERP were then added to the culture at the concentration of 30 μg/mL. At 24 h post siRNA treatment, cells were supplemented with 10% FBS. Culture medium were removed at 72 h post siRNA treatment and the cells were lysed with QuantiGene Lysis Mixture for next step gene expression quantification.

### QuantiGene Branched DNA Assay

To evaluate gene silencing activity, cell lysates collected after siRNA treatment were subject to QuantiGene branched DNA assay according to manufacturer's protocol. In brief, cell lysates were incubated with capture probes targeting human PPIB (target gene) and GAPDH (endogenous control) at 55 °C for 18-20 h. After washing, the plates were incubated with pre-amplification probes and amplification probes to amplify the signal. The excessive probes were then washed off and assay substrates were added to allow quantifying luminescence using a plate reader. The signal from PPIB was normalized to the signal from GAPDH.

### Results

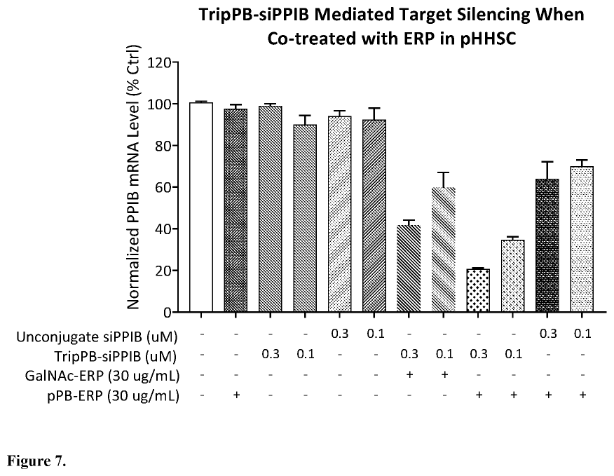

When combined with 30 μg/mL of pPB-ERP, TripPB-siPPIB enabled 79% and 65% target gene KD when treated at 0.3 and 0.1 μM, respectively (FIG. 7). In contrast, unconjugated siPPIB showed low gene silencing activity (<40%) when treated in combination with 30 μg/mL pPB-ERP and the dose response effect was not appreciable. RNAi activity was also observed in the TripPB-siPPIB+GalNAc-ERP combo treatment group but the GalNAc-ERP was less effective as compared to pPB-ERP. siRNA alone, either unconjugated or conjugated with TripPB, or pPB-ERP alone showed no effect on target gene silencing. These results suggested that pPB ligand could improve both siRNA and ERP delivery to pHHSC in vitro.

### Example 11. pPB-ERP Improves In Vivo Gene Silencing by TripPB siRNA Conjugate in Mouse Liver Fibrosis Model

HSC specific gene Hsp47 was selected as the siRNA target to allow evaluation of RNAi activity in the targeted cell population.

### Materials and Methods

Assessment of TripPB siRNA Conjugate and pPB-ERP in Mouse Liver Fibrosis Model

Balb/c female mice aged 6-8 weeks (n=4-5 per group) were injected intraperitoneally with 2.5 mL/kg of 20% CCl4 in corn oil twice a week for two weeks to induce HSC activation. One day post the last dose of CCl4, animals were injected subcutaneously (SQ) in the scapular region with a single dose of vehicle control (saline), pPB-ERP alone (40 mg/kg), TripPB-siHsp47 (10 mg/kg) with pPB-ERP (40 mg/kg), unconjugated siHsp47 (10 mg/kg) with pPB-ERP (40 mg/kg), using a volume of 5 mL/kg body weight. Single intravenous (IV) dosing of TripPB-siHsp47 (10 mg/kg) with pPB-ERP (40 mg/kg) was also tested in one group of the animals. The livers of treated animals were collected two days post siRNA and ERP treatment and fixed with RNAlater for a minimum of 16 h at 4 °C. RNAi activity of siRNA conjugates was assessed by quantifying target gene expression using Quantigene assay.

### QuantiGene Branched DNA Assay

Between 20 and 25 mg of RNAlater fixed liver tissue was homogenized in Epicentre lysis buffer containing 1% proteinase K using the FastPrep®-24 homogenization instrument (4.0 m/s, 3×15 second bursts, MP Biomedicals). Samples were kept on ice both before and after the homogenization to prevent degradation. The lysates were cleared by centrifugation at 16,000×g at 16 °C for 5 min and diluted in lysis working buffer to ensure all values would be in the assay linear range of detection before being subjected to bDNA assay using the Affymetrix QuantiGene 2.0 assay kit, according to the manufacturer's protocol. The bDNA probes were specifically designed to target mouse Hsp47 and mouse Gapdh. The signal from Hsp47 was normalized to the signal from Gapdh.

### Results

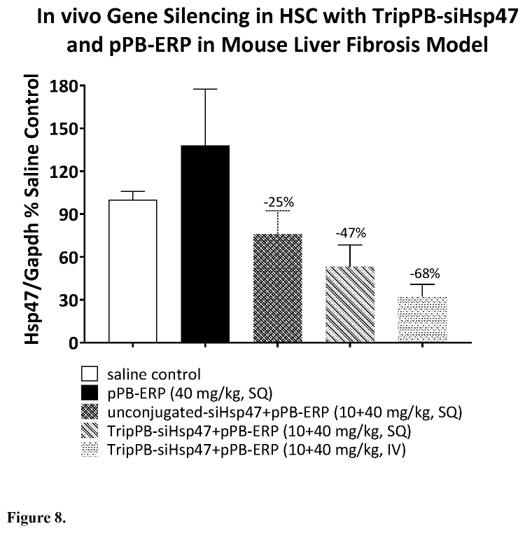

Combination treatment of TripPB-siHsp47 with pPB-ERP in the tested dosing regimen resulted in 47% target gene KD when dosed subcutaneously (FIG. 8) and 68% gene suppression when dosed intravenously. The improvement of RNAi activity with IV dosing is likely associated with high bioavailability with this dosing method. In contrast, minimal (25%) gene silencing was observed in animals treated with unconjugated siHsp47 and pPB-ERP. pPB-ERP treatment alone did not induce any reduction in target gene expression. Of note, Hsp47 is an HSC specific gene. Together, these data demonstrated that trivalent pPB ligand conjugated siRNA in combination with pPB-ERP could enable target gene silencing in HSC in liver fibrosis model.

### Example 12. Dose Response of pPB-ERP in HSC Targeted RNAi in Mouse Liver Fibrosis Model

HSC specific gene Hsp47 was selected as the siRNA target to allow evaluation of RNAi activity in the targeted cell population.

### Materials and Methods

Assessment of pPB-ERP Dose Response in Mouse Liver Fibrosis Model

Balb/c female mice aged 6-8 weeks (n=4-5 per group) were injected intraperitoneally with 2.5 mL/kg of 20% CCl4 in corn oil twice a week for two weeks to induce HSC activation. One day post the last dose of CCl4, animals were injected subcutaneously in the scapular region with a single dose of vehicle control (saline), TripPB-siHsp47 (3 mg/kg), pPB-ERP alone (40 mg/kg) or TripPB-siHsp47 (3 mg/kg) with pPB-ERP at 12, 20 or 40 mg/kg, using a volume of 5 mL/kg body weight. The livers of treated animals were collected two days post siRNA and ERP treatment and fixed with RNAlater for a minimum of 16 h at 4 °C. RNAi activity of siRNA conjugates was assessed by quantifying target gene expression using Quantigene assay.

QuantiGene branched DNA Assay

Between 20 and 25 mg of RNAlater fixed liver tissue was homogenized in Epicentre lysis buffer containing 1% proteinase K using the FastPrep®-24 homogenization instrument (4.0 m/s, 3×15 second bursts, MP Biomedicals). Samples were kept on ice both before and after the homogenization to prevent degradation. The lysates were cleared by centrifugation at 16,000×g at 16 °C for 5 min and diluted in lysis working buffer to ensure all values would be in the assay linear range of detection before being subjected to bDNA assay using the Affymetrix QuantiGene 2.0 assay kit, according to the manufacturer's protocol. The bDNA probes were specifically designed to target mouse Hsp47 and mouse Gapdh. The signal from Hsp47 was normalized to the signal from Gapdh.

### Results

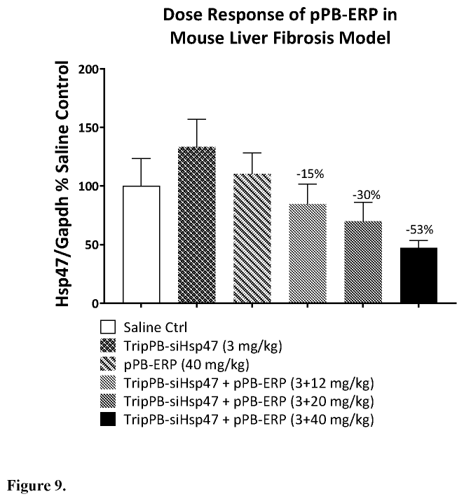

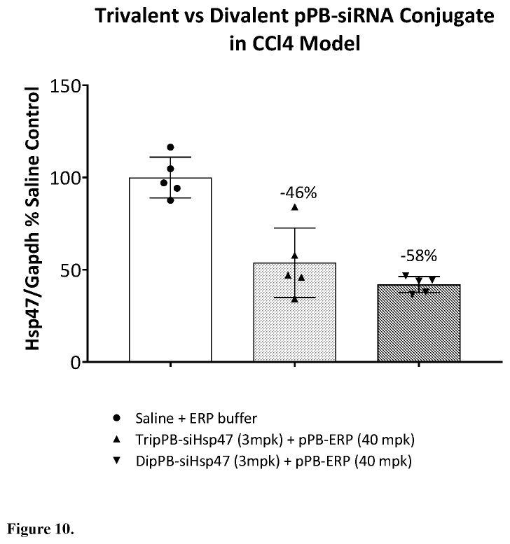

No gene silencing was observed in the TripPB-siHsp47 or pPB-ERP single treatment groups (FIG. 9) which is consistent with results in other examples reported in this patent. Dose response effect in pPB-ERP treatment was evident in the TripPB-siHsp47 combination treatment groups, with 12, 20, and 40 mg/kg of pPB-ERP resulted in 15%, 30% and 53% decline in the gene expression of HSC target Hsp47. These data demonstrated the importance of including an endosome release element.

### Example 13. Divalent pPB-siRNA Conjugates Showed Comparable Activity to TripPB siRNA Conjugate in Mouse Liver Fibrosis Model

This example illustrates the in vivo gene silencing mediated by divalent and trivalent pPB conjugate siRNA together with pPB-ERP in a CCl4 induced mouse liver fibrosis model. HSC specific gene Hsp47 was selected as the siRNA target to allow evaluation of RNAi activity in the targeted cell population.

### Materials and Methods

Assessment of DipPB and TripPB siRNA Conjugate and pPB-ERP in Mouse Liver Fibrosis Model

Balb/c female mice aged 6-8 weeks (n=4-5 per group) were injected intraperitoneally with 2.5 mL/kg of 20% CCl4 in corn oil twice a week for two weeks to induce HSC activation. One day post the last dose of CCl4, animals were injected subcutaneously (SQ) in the scapular region with a single dose of vehicle control (saline), DipPB-siHsp47 (3 mg/kg) with pPB-ERP (40 mg/kg), or TripPB-siHsp47 (3 mg/kg) with pPB-ERP (40 mg/kg), using a volume of 5 mL/kg body weight. The livers of treated animals were collected two days post siRNA and ERP treatment and fixed with RNAlater for a minimum of 16 h at 4 °C. RNAi activity of siRNA conjugates was assessed by quantifying target gene expression using Quantigene assay.

### QuantiGene Branched DNA Assay

Between 20 and 25 mg of RNAlater fixed liver tissue was homogenized in Epicentre lysis buffer containing 1% proteinase K using the FastPrep®-24 homogenization instrument (4.0 m/s, 3×15 second bursts, MP Biomedicals). Samples were kept on ice both before and after the homogenization to prevent degradation. The lysates were cleared by centrifugation at 16,000×g at 16 °C for 5 min and diluted in lysis working buffer to ensure all values would be in the assay linear range of detection before being subjected to bDNA assay using the Affymetrix QuantiGene 2.0 assay kit, according to the manufacturer's protocol. The bDNA probes were specifically designed to target mouse Hsp47 and mouse Gapdh. The signal from Hsp47 was normalized to the signal from Gapdh.

### Results

Combination treatment of TripPB-siHsp47 with pPB-ERP in the tested dosing regimen showed 46% target gene KD. Replacing TripPB-siHsp47 with DipPB-siHsp47 resulted in 58% gene silencing but there were no statistical differences to the TripPB-siHsp47 group. Overall, the trivalent and divalent pPB-siRNA conjugates showed comparable activity in this study.

### Synthesis of Endosome Release Polymer

Endosome release polymers (Mannose-ERP 40 and GalNAc-ERP 41) were synthesized by Syngene International LTD using synthetic methodology analogous to that described in Prieve, M. G., Harvie, P., Monahan, S. D., Roy, D., Li, A. G., Blevins, T. L., Paschal, A. E., Waldheim, M., Bell, E. C., Galperin, A., Ella-Menye, J. R., et al. (2018). Targeted mRNA Therapy for Ornithine Transcarbamylase Deficiency. Mol Ther 26, 801-813. 10.1016/j.ymthe.2017.12.024. The structures of Mannose-ERP 40 and GalNAc-ERP 41 are presented below.

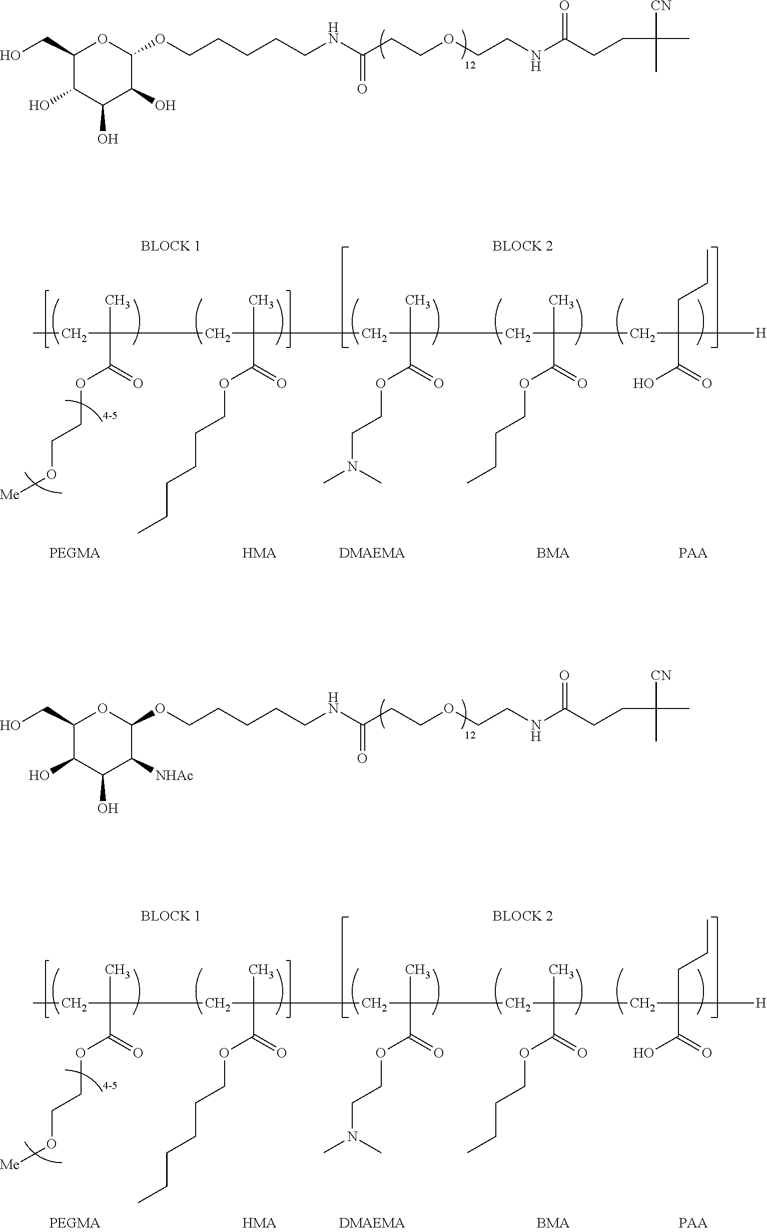

Block 1 MW 5.7 KDa 15% of Target value (4.85-6.56 KDa)
Block 2 MW 7.9 kDa 15% of target value (9.11-6.73 kDa)
Block 1 Monomer Incorporation
   PEGMA 75% (71-79%)
   HMA 25% (21-29%)
Block 2 Monomer Incorporation
   BMA 54% (47-57%)
   DMAEMA 35% (30-40%)
   PAA 12% (9-16%)

What is claimed is:

1. A compound of formula (I)
(R¹)ₓ-L-(R²)_{y} (I)
or a salt thereof, wherein:
x is 2, 3, 4 or 5;
each R¹ is independently a targeting ligand, which is selected from:
a) a cyclic polypeptide depicted by SEQ ID NO: 1:C*SRNLIDC* (SEQ ID NO: 1), wherein * denotes a disulfide bond linking the two C residues to thereby form the cyclic polypeptide, and
b) any cyclic polypeptide having at least 80% sequence identity with the polypeptide depicted by SEQ ID NO: 1, under the proviso that both C* residues are present, and wherein * denotes a disulfide bond linking the two C residues to thereby form the cyclic polypeptide;
L is a linking group;
y is 1, 2, 3, 4 or 5;
and each R² is independently an oligonucleotide, a label (e.g., a label derivable from fluorescein isothiocyanate (FITC) or Cy5), a phenyl group that is substituted with a formyl (—CHO) group, or a group of formula:

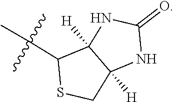 [SMILES: *C1SC[C@@H]2NC(=O)N[C@H]12]

2. The compound or salt of claim 1, wherein x is 2 or 3.

3. The compound or salt of claim 1, wherein x is 3.

4. The compound or salt of any one of claims 1-3, wherein each R¹ is covalently bound to L (i) through the N terminus of the polypeptide or (ii) through the C terminus of the polypeptide or (iii) through the side chain of an amino acid of the polypeptide.

5. The compound or salt of any one of claims 1-3, wherein each R¹ is covalently bound to L through the N terminus of the polypeptide.

6. The compound or salt of any one of claims 1-3, wherein each R¹ is independently a targeting ligand of formula:

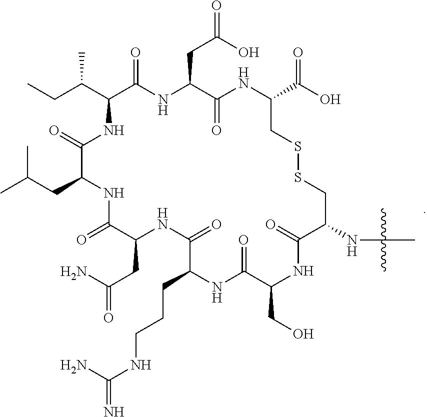 [SMILES: *N[C@H]1CSSC[C@@H](C(=O)O)NC(=O)[C@H](CC(=O)O)NC(=O)[C@H]([C@@H](C)CC)NC(=O)[C@H](CC(C)C)NC(=O)[C@H](CC(N)=O)NC(=O)[C@H](CCCNC(=N)N)NC(=O)[C@H](CO)NC1=O]

7. The compound or salt of any one of claims 1-6, wherein each R² is independently an oligonucleotide.

8. The compound or salt of any one of claims 1-6, wherein each R² is independently an siRNA.

9. The compound or salt of any one of claims 7-8, wherein each oligonucleotide or each siRNA is attached to L through a phosphate P(═O)(OH)₂—O— of each oligonucleotide or each siRNA.

10. The compound or salt of any one of claims 1-6, wherein each R² is independently a group of formula:

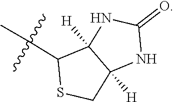 [SMILES: *C1SC[C@@H]2NC(=O)N[C@H]12]

11. The compound or salt of any one of claims 1-6, wherein each R² is independently 3-formylphenyl or 4-formylphenyl.

12. The compound or salt of any one of claims 1-11, wherein L is a branched or unbranched, saturated or unsaturated, hydrocarbon chain, having from about 1 to about 500 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

13. The compound or salt of any one of claims 1-11, wherein L is a branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, azido, cyano, nitro, halo, —N(R^{a})₂, hydroxy, oxo (═O), and carboxy, wherein each R^{a} is independently H or (C₁-C₆)alkyl.

14. The compound or salt of any one of claims 1-11, wherein L is a branched or unbranched, saturated or unsaturated, hydrocarbon chain, having from about 1 to about 100 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

15. The compound or salt of any one of claims 1-11, wherein L is a branched or unbranched, saturated or unsaturated, hydrocarbon chain, having from about 1 to about 50 carbon atoms, wherein one or more of the carbon atoms is optionally replaced independently by —O—, —S, —N(R^{a})—, 3-20 membered heterocycle, 3-20 membered heteroaryl, 6-14 membered aryl, or 3-20 membered carbocycle and wherein each chain, 3-20 membered heterocycle, 3-20 membered heteroaryl, 6-14 membered aryl, and 3-20 membered carbocycle is optionally and independently substituted with one or more (e.g. 1, 2, 3, 4, 5 or more) substituents independently selected from the group consisting of azido, halo, and oxo (═O), and carboxy, wherein each R^{a} is independently H or (C₁-C₆)alkyl.

16. The compound or salt of any one of claims 1-11, wherein L is a branched or unbranched, saturated or unsaturated, hydrocarbon chain, having from about 1 to about 50 carbon atoms, wherein one or more of the carbon atoms is optionally replaced independently by —O—, —S, —N(R^{a})—, or R^{b}, wherein each chain and R^{b} is optionally and independently substituted with one or more (e.g. 1, 2, 3, 4, 5 or more) substituents independently selected from the group consisting of azido, halo, ═N(OH), —O(NH2) and oxo (═O), and carboxy, wherein each R^{b} is independently H or (C₁-C₆)alkyl; and each R^{b} is independently selected from the group consisting of:

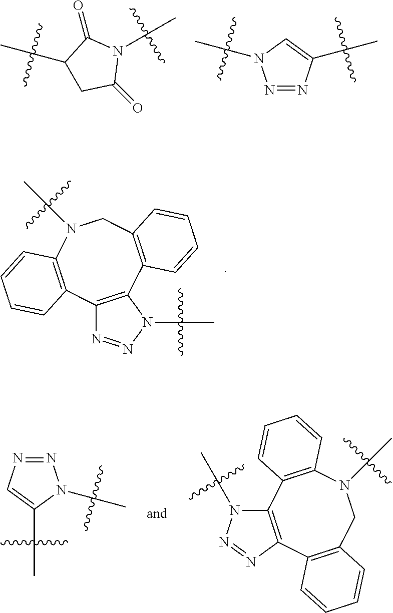 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

17. The compound or salt of any one of claims 1-11, wherein L comprises: —C(═O)(CH₂)ₐN(H)C(═O)—; wherein a is 3, 4, 5, 6, 7, or 8.

18. The compound or salt of any one of claims 1-11, wherein L comprises —(CH₂CH₂O)_{b}CH₂CH₂C(═O)— or —(CH₂CH₂O)_{b}CH₂CH₂N(H)C(═O)—, wherein b is 2-50.

19. The compound or salt of any one of claims 1-11, wherein L comprises phen-1,3-diyl or phen1-1,3,5-triyl.

20. The compound or salt of any one of claims 1-11, wherein L comprises:

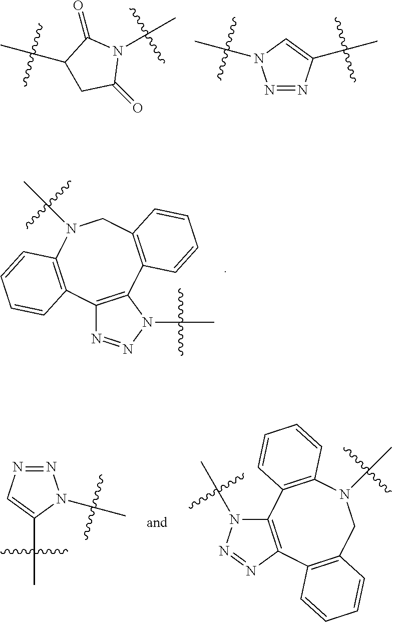 [SMILES: *C1CC(=O)N(*)C1=O ; *c1cn(*)nn1 ; *N1Cc2ccccc2-c2c(nnn2*)-c2ccccc21 ; *c1cnnn1* ; *N1Cc2ccccc2-c2nnn(*)c2-c2ccccc21]

21. The compound or salt of any one of claims 1-20, wherein each R¹ is linked to L through a carbonyl group of L.

22. The compound or salt of any one of claims 1-20, wherein each R² is linked to L through a carbon-carbon bond.

23. The compound or salt of any one of claims 1-11, wherein L is selected from the group consisting of:

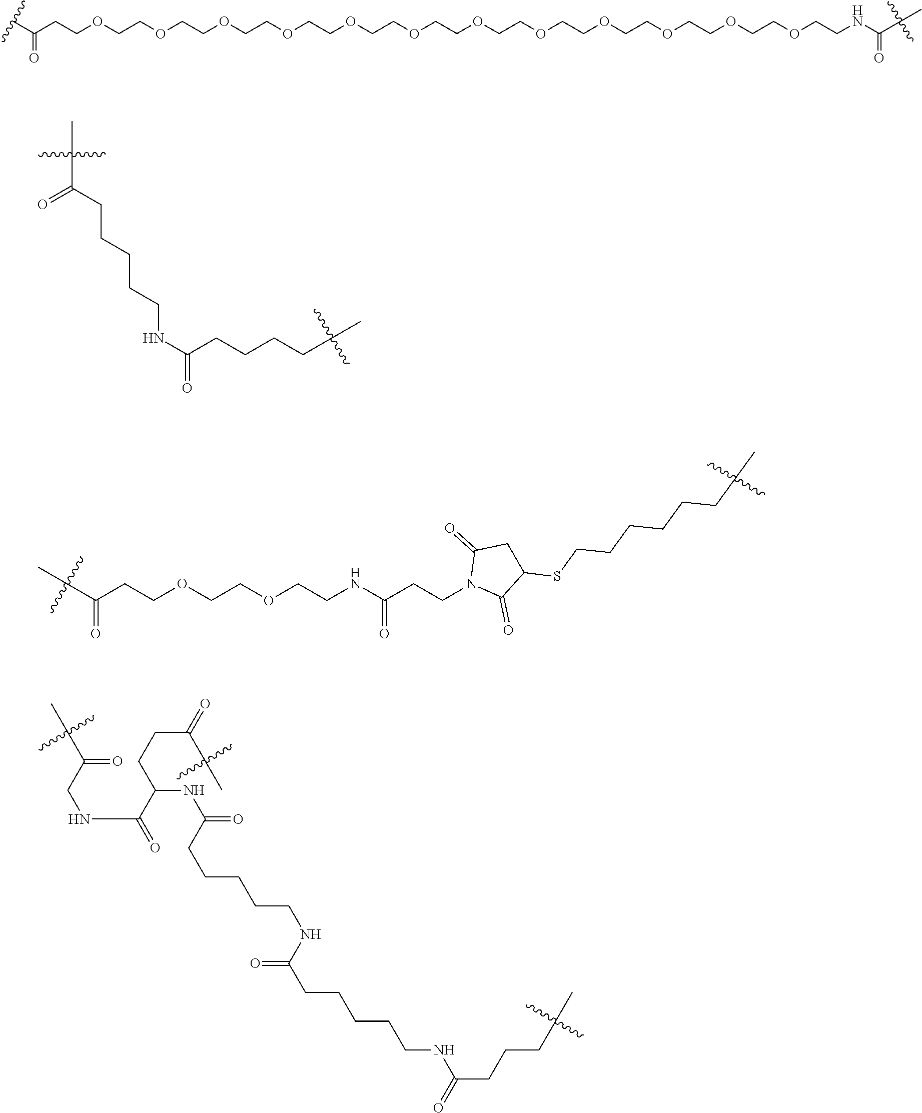 [SMILES: *C(=O)CCOCCOCCOCCOCCOCCOCCOCCOCCOCCOCCOCCOCCNC(*)=O ; *CCCCC(=O)NCCCCCC(*)=O ; *CCCCCCSC1CC(=O)N(CCC(=O)NCCOCCOCCC(*)=O)C1=O ; *CCCC(=O)NCCCCCC(=O)NCCCCCC(=O)NC(CCC(*)=O)C(=O)NCC(*)=O]

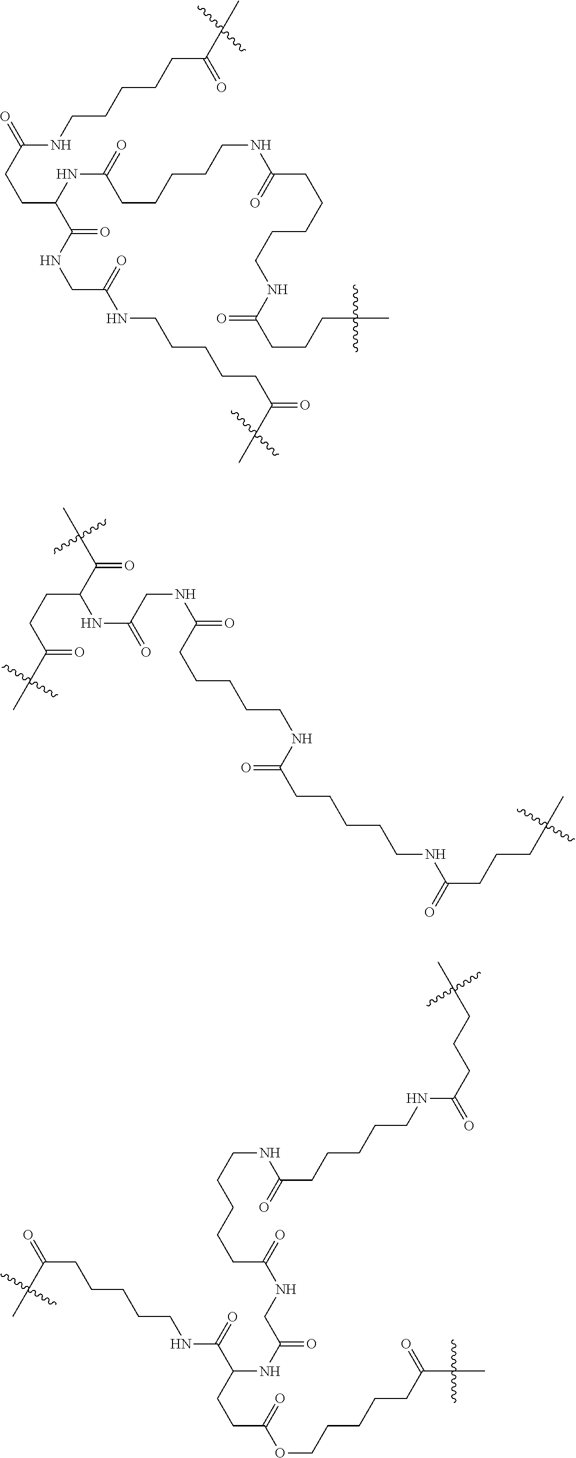 [SMILES: *CCCC(=O)NCCCCCC(=O)NCCCCCC(=O)NC(CCC(=O)NCCCCCC(*)=O)C(=O)NCC(=O)NCCCCCC(*)=O ; *CCCC(=O)NCCCCCC(=O)NCCCCCC(=O)NCC(=O)NC(CCC(*)=O)C(*)=O ; *CCCC(=O)NCCCCCC(=O)NCCCCCC(=O)NCC(=O)NC(CCC(=O)OCCCCCC(*)=O)C(=O)NCCCCCC(*)=O]

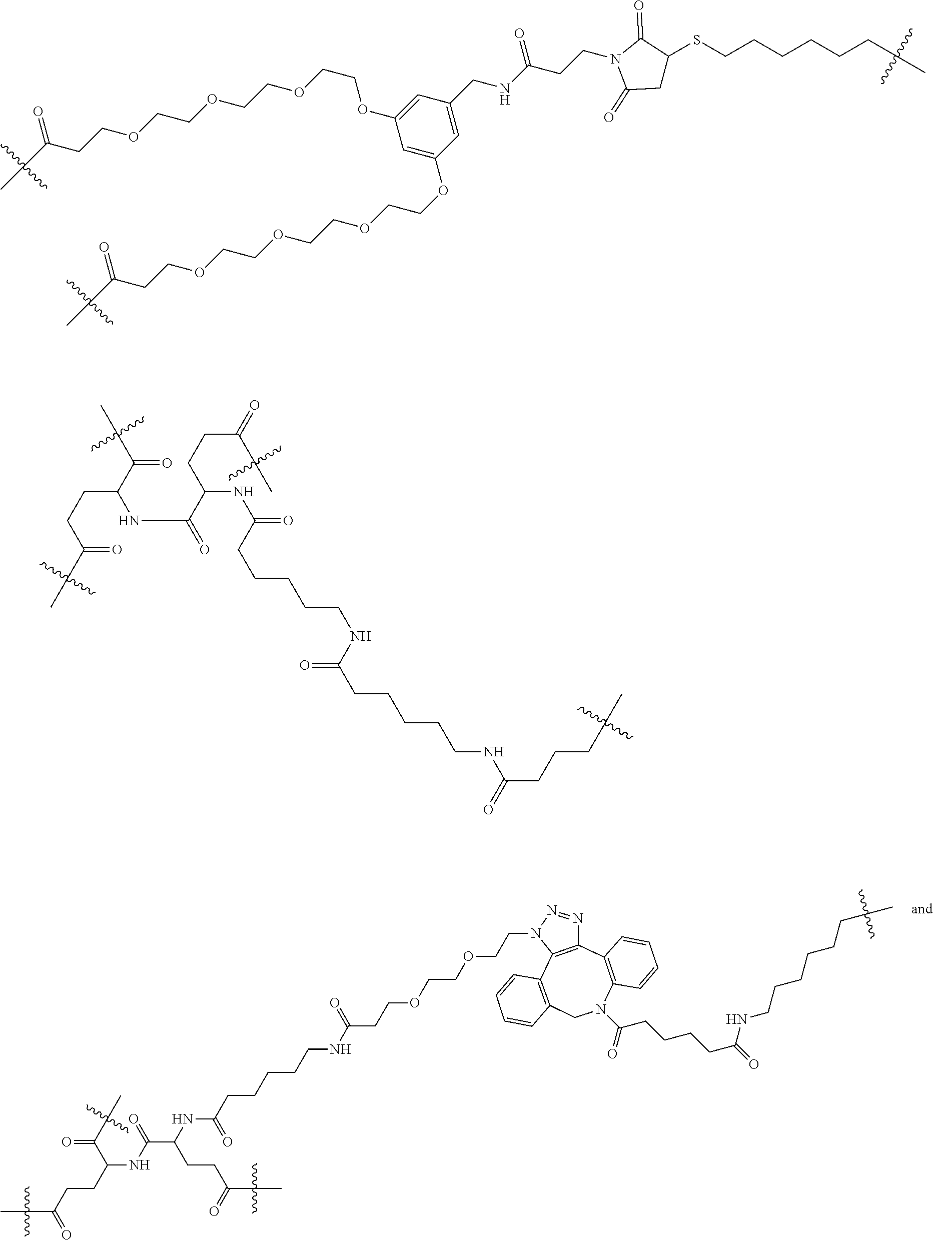 [SMILES: *CCCCCCSC1CC(=O)N(CCC(=O)NCc2cc(OCCOCCOCCOCCC(*)=O)cc(OCCOCCOCCOCCC(*)=O)c2)C1=O ; *CCCC(=O)NCCCCCC(=O)NCCCCCC(=O)NC(CCC(*)=O)C(=O)NC(CCC(*)=O)C(*)=O ; *CCCCCCNC(=O)CCCCC(=O)N1Cc2ccccc2-c2c(nnn2CCOCCOCCC(=O)NCCCCCC(=O)NC(CCC(*)=O)C(=O)NC(CCC(*)=O)C(*)=O)-c2ccccc21]

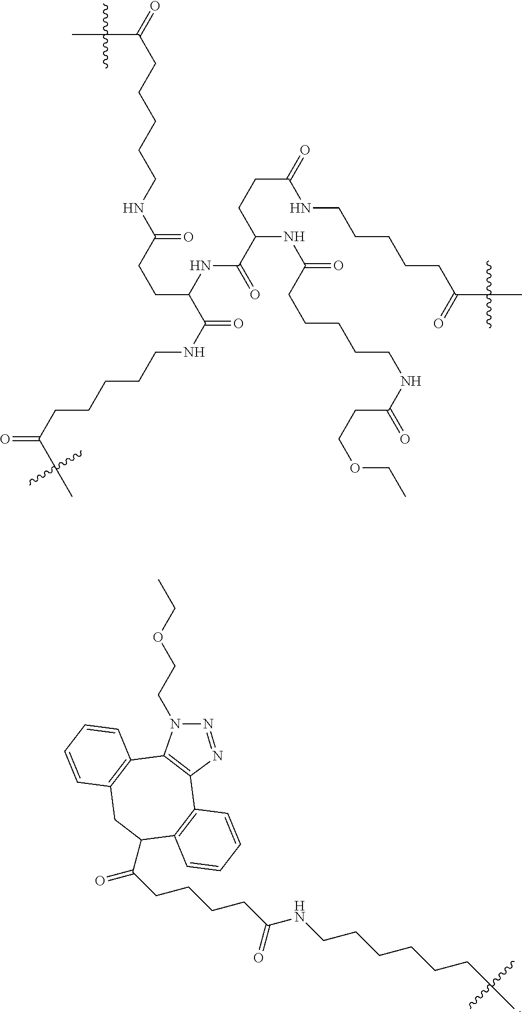 [SMILES: *C(=O)CCCCCNC(=O)CCC(NC(=O)CCCCCNC(=O)CCOCC)C(=O)NC(CCC(=O)NCCCCCC(*)=O)C(=O)NCCCCCC(*)=O ; *CCCCCCNC(=O)CCCCC(=O)C1Cc2ccccc2-c2c(nnn2CCOCC)-c2ccccc21]

24. The compound essentially as defined in claim 1, wherein x is 1, 2 or 3, wherein the compound is selected from the group consisting of:

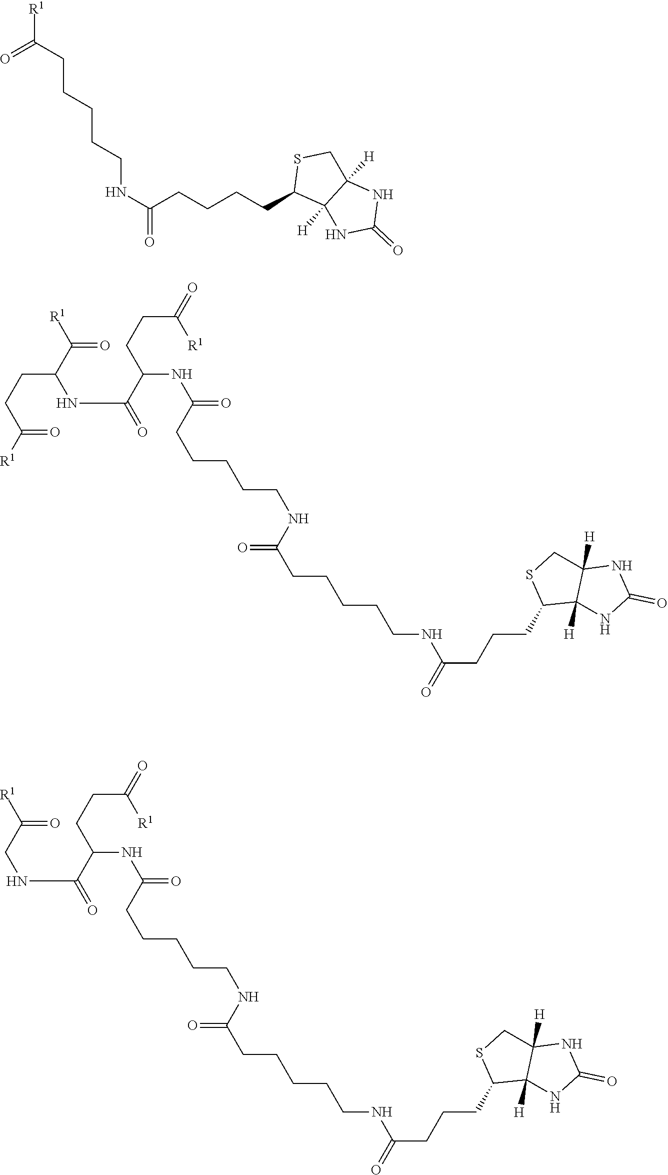

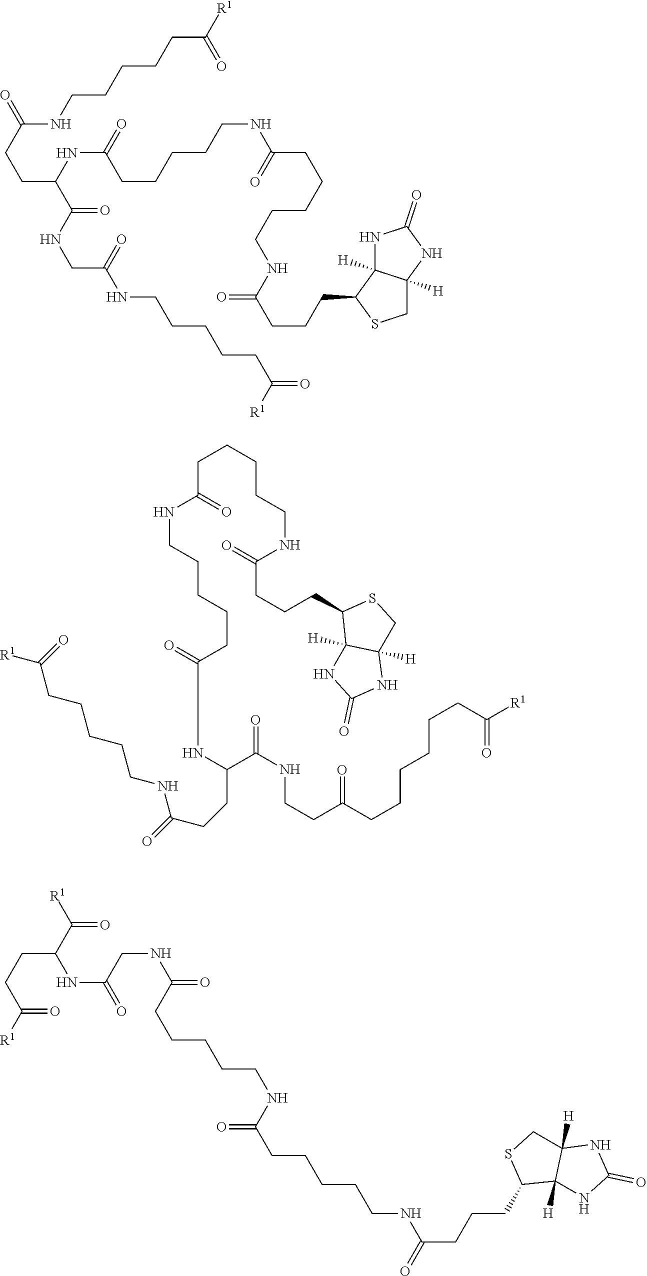

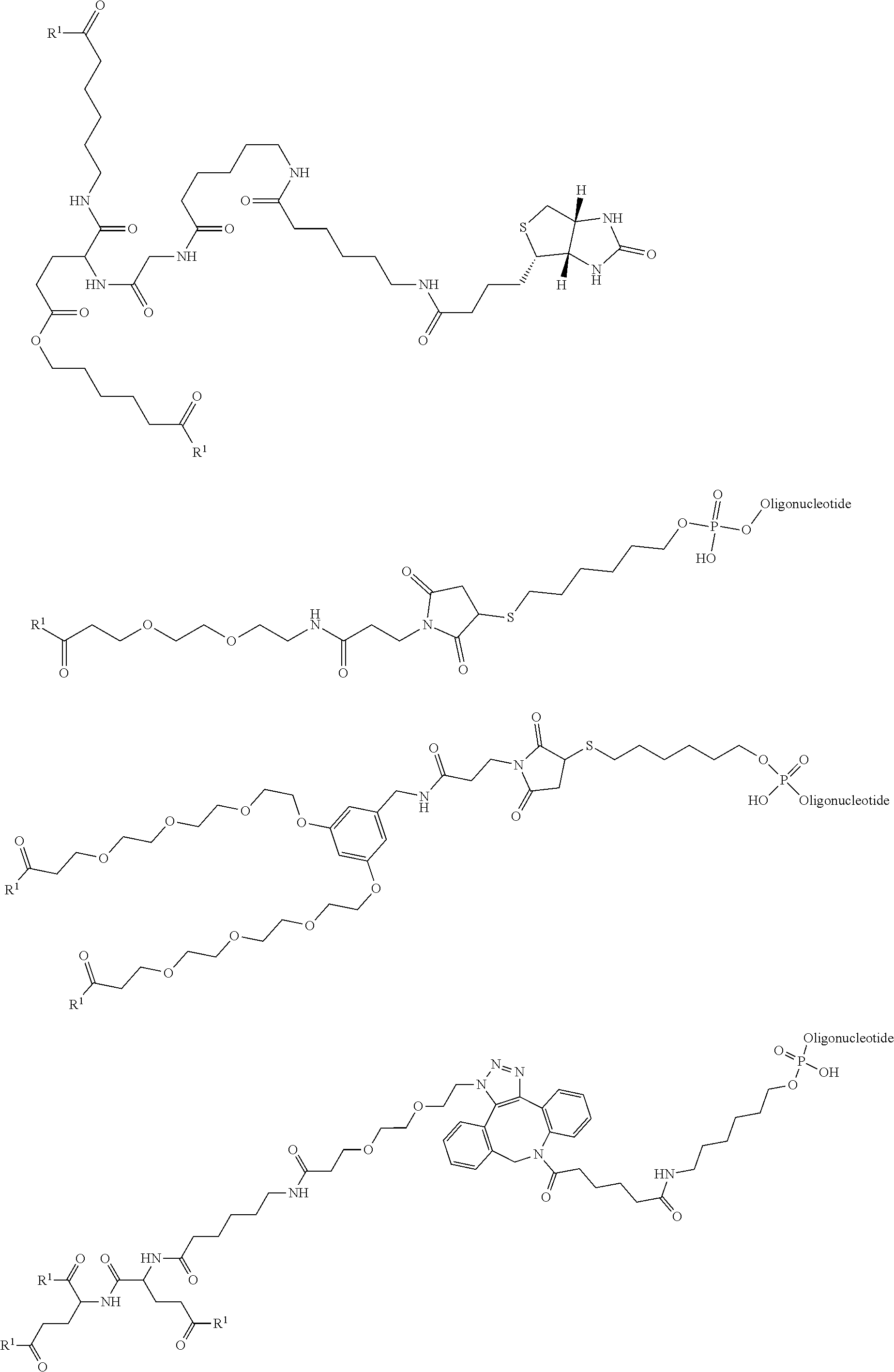

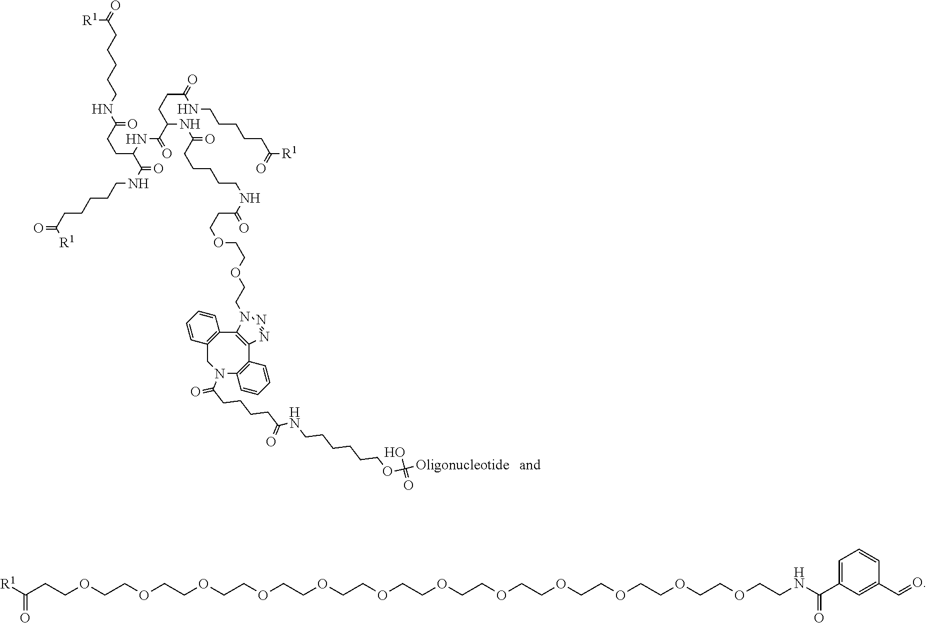

25. A process for preparation of a diblock polymer of the following formula (X): 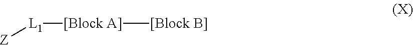
comprising:
a) contacting a compound of structure Va, Vb, Vc, or Vd 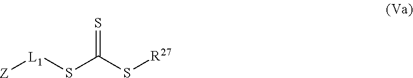
wherein R²⁷ is (C₁-C₁₂)alkyl, 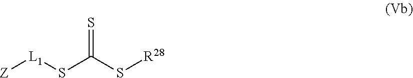
wherein R²⁸ is (C₁-C₁₂)alkyl, 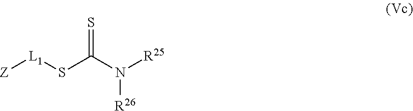
wherein R²⁵ and R²⁶ are independently H, (C₁-C₁₂)alkyl, aryl, or heteroaryl, 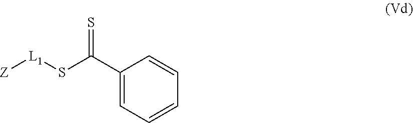
with one or more A monomers selected from the group consisting of:
iii) a polyethyleneglycol methacrylate with 2-20 ethylene glycol units (PEGMA); and
iv) M², wherein M² is a methacrylate, which is optionally selected among
a (C₄-C₁₈)alkyl-methacrylate;
a (C₄-C₁₈)branched alkyl-methacrylate;
a cholesteryl methacrylate;
a (C₄-C₁₈)alkyl-methacrylate substituted with one or more fluorine atoms; and
a (C₄-C₁₈)branched alkyl-methacrylate substituted with one or more fluorine atoms;
in the presence of a free radical to provide a first product;
b) contacting the first product with one or more B monomers of formulae B1, B2 and B3 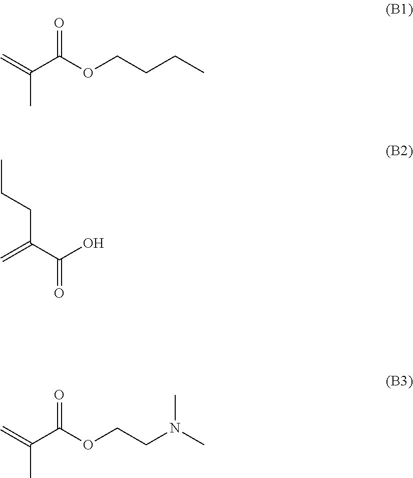
in the presence of a free radical to provide a second product, and
c) optionally contacting the second product with a free radical source (e.g., AIBN) to remove the chain transfer agent and provide the diblock polymer of formula (X);
wherein:
Block A comprises one or more residues of A monomers and has a molecular weight of from about 1 kDa to about 25 kDa;
Block B comprises one or more residues of monomers B1, B2, and B3 and has a molecular weight of from about 1 kDa to about 25 kDa.
L₁ is a linking moiety;
Z is a functional group that is optionally protected with a protecting group and is capable of reacting with Y of a compound of formula (XII):
T-L₂-Y (XII)
to form a conjugate wherein:
T is a ligand (optionally targeting ligand);
Y is a functional group that is capable of reacting with Z to form a conjugate; and
L₂ is absent or is a linking moiety.

26. The process of claim 25, wherein the A monomers are selected from the group consisting of monomers of formulae A1 and A2,
PEGMA is 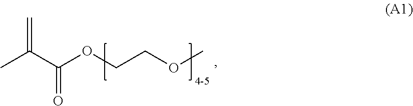
and
M² is

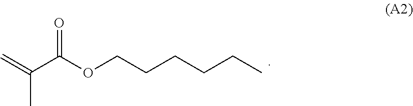

27. The process of claim 25 or 26 wherein Z is a functional group that is protected with a protecting group, wherein the process further comprises removing the protecting group from Z.

28. The process of claim 25 or 26 wherein Z is a functional group that is not protected with a protecting group.

29. The process of claim 27 or 28 further comprising reacting the diblock polymer of formula (X) with a compound of formula (XII):
T-L₂-Y (XII)
under conditions where Z reacts with Y to form a group Y′Z′ and yield a conjugate of formula:
T-L₂-Y′Z′-L₁[Block A]-[Block B].

30. The process of claim 29, wherein Block A has a molecular weight of from about 1 kDa to about 25 kDa; and Block B has a molecular weight of from about 1 kDa to about 25 kDa.

31. The process of claim 29, wherein Block A has formula:
-[PEGMAₘ-M²ₙ]ᵥ-
wherein:
PEGMA is polyethyleneglycol methacrylate residue with 2-20 ethylene glycol units; M² is a methacrylate residue selected from the group consisting of
a (C₄-C₁₈)alkyl-methacrylate residue;
a (C₄-C₁₈)branched alkyl-methacrylate residue;
a cholesteryl methacrylate residue;
a (C₄-C₁₈)alkyl-methacrylate residue substituted with one or more fluorine atoms; and
a (C₄-C₁₈)branched alkyl-methacrylate residue substituted with one or more fluorine atoms; and
v is 1 to 25 kDa;
and wherein block B has formula:
-[DMAEMA_{q}-PAAᵣ-BMAₛ]_{w}
wherein:
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1; and
w is 1 to 25 kDa;

32. The process of any one of claims 25-31, wherein Z and Y are selected so that Y′Z′ comprises an oxime functionality.

33. The process of any one of claims 25-31, wherein Z and Y are selected so that Y′Z′ comprises a triazole ring.

34. The process of any one of claims 25-27 and 29-32, wherein Z comprises an aminooxy group that is protected and the protecting group is on the nitrogen of the aminooxy group.

35. The process of any one of claims 29-32, wherein Z comprises an aminooxy group, Y comprises a ketone group or an aldehyde group, and Y′Z′ comprises an oxime functional group.

36. The process of any one of claims 29-32, wherein Z comprises a ketone group or an aldehyde group, Y comprises an aminooxy group, and Y′Z′ comprises an oxime functional group.

37. The process of claim 29 or 33, wherein Z comprises an alkyne group, Y comprises an azide group, and Y′Z′ comprises a triazole ring.

38. The process of claim 29 or 33, wherein Z comprises an azide group, Y comprises an alkyne group, and Y′Z′ comprises a triazole ring.

39. The process of any one of claims 29-38 wherein T is a targeting ligand or a labeling agent.

40. The process of any one of claims 29-38, wherein the targeting ligand is selected from the group of an oligonucleotide, a peptide, a saccharide, and a small molecule.

41. The process of any one of claims 29-38, wherein the targeting ligand comprises a cyclic peptide.

42. The process of any one of claims 29-38, wherein the targeting ligand comprises a cyclic peptide as described in claim 1.

43. The process of any one of claims 29-38, wherein the targeting ligand comprises a cyclic peptide as described in claim 6.

44. The process of any one of claims 29-38, wherein the labeling agent is selected from the group consisting of a fluorophore, a chromophore, and a radionucleotide.

45. The process of any one of claims 29-38, wherein the labeling agent is derivable from fluorescein isothiocyanate (FITC) or Cy5.

46. A product prepared by the process of any one of claims 25-45.

47. A conjugate prepared by the process of any one of claims 29-45.

48. A composition comprising a plurality of conjugates as defined in any one of claims 29-45.

49. The composition of claim 48, wherein at least one of the conjugates comprises a labelling agent.

50. A compound of formula (I)
(R¹)ₓ-L-(R²)ᵥ (I)
or a salt thereof, wherein:
x is 1, 2, 3, 4 or 5;
each R¹ is independently a targeting ligand, which is selected from
a) a cyclic polypeptide depicted by SEQ ID NO: 1:C*SRNLIDC* (SEQ ID NO: 1), wherein * denotes a disulfide bond linking the two C residues to thereby form the cyclic polypeptide,
b) any cyclic polypeptide having at least 80% sequence identity with the polypeptide depicted by SEQ ID NO: 1, under the proviso that both C* residues are present, and wherein * denotes a disulfide bond linking the two C residues to thereby form the cyclic polypeptide;
L is a linking group; and
y is 1, 2, 3, 4 or 5;
and each R² is independently an endosomal release polymer.

51. The compound or salt of claim 50, wherein the endosomal release polymer comprises a diblock polymer comprising Block A and Block B having the following formula:
[Block A]-[Block B]
wherein:
Block A comprises one or more residues of the A monomers described in claim 25 and has a molecular weight of from about 1 kDa to about 25 kDa; and
Block B comprises one or more residues of monomers B1, B2, and B3 as described in claim 25 and has a molecular weight of from about 1 kDa to about 25 kDa.

52. The compound or salt of claim 50, wherein the endosomal release polymer has formula (XX):
-[PEGMAₘ-M²ₙ]v-[DMAEMA_{q}-PAAᵣ-BMAₛ]_{w} (XX)
wherein:
PEGMA is polyethyleneglycol methacrylate residue with 2-20 ethylene glycol units; M² is a methacrylate residue selected from the group consisting of
a (C₄-C₁₈)alkyl-methacrylate residue;
a (C₄-C₁₈)branched alkyl-methacrylate residue;
a cholesteryl methacrylate residue;
a (C₄-C₁₈)alkyl-methacrylate residue substituted with one or more fluorine atoms; and
a (C₄-C₁₈)branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa; and
w is 1 to 25 kDa.

53. The compound or salt of claim 51, wherein Block A comprises one or more residues of monomers of formulae A1 and A2 as described in claim 26.

54. A pharmaceutical composition comprising a compound as described in any one of claims 1-24 and 50-53 or a pharmaceutically acceptable salt thereof, and a pharmaceutically acceptable carrier.

55. A pharmaceutical composition comprising a first compound as described in any one of claims 1-24 or a pharmaceutically acceptable salt thereof, wherein each R² is independently an oligonucleotide and a second compound as described in any one of claims 50-53 or a pharmaceutically acceptable salt thereof.

56. A method to deliver an oligonucleotide to an animal comprising administering a compound as described in any one of claims 1-24 or a pharmaceutically acceptable salt thereof, to the animal.

57. A method to deliver an oligonucleotide to cells that express platelet-derived growth factor receptor (PDGFR) in an animal comprising administering a compound as described in any one of claims 1-24 or a pharmaceutically acceptable salt thereof, to the animal.

58. The method of claim 57, wherein the platelet-derived growth factor receptor (PDGFR) is platelet-derived growth factor receptor alpha (PDGFRα).

59. The method of claim 57, wherein the platelet-derived growth factor receptor (PDGFR) is platelet-derived growth factor receptor beta (PDGFRβ).

60. The method of claim 57, wherein the cells that express PDGFR are hepatic stellate cells (HSC), endothelial cells, fibroblasts or tumor cells.

61. A method to treat a disease involving PDGFR expressing cells (e.g., liver fibrosis, non-alcoholic steatohepatitis (NASH), clear cell renal cell carcinoma, kidney fibrosis, or alcoholic steatohepatitis (ASH)) comprising administering a compound as described in any one of claims 1-24 or a pharmaceutically acceptable salt thereof, to the animal.

62. A compound as described in any one of claims 1-24 or a pharmaceutically acceptable salt thereof for delivering an oligonucleotide to an animal.

63. A compound as described in any one of claims 1-24 or a pharmaceutically acceptable salt thereof for delivering an oligonucleotide to cells that express platelet-derived growth factor receptor (PDGFR).

64. A compound as described in any one of claims 1-24 or a pharmaceutically acceptable salt thereof for the prophylactic or therapeutic treatment of a disease involving PDGFR expressing cells (e.g., liver fibrosis, non-alcoholic steatohepatitis (NASH), clear cell renal cell carcinoma, kidney fibrosis, or alcoholic steatohepatitis (ASH)).

65. The use of a compound as described in any one of claims 1-24 or a pharmaceutically acceptable salt thereof to prepare a medicament for delivering an oligonucleotide to an animal.

66. The use of a compound as described in any one of claims 1-24 or a pharmaceutically acceptable salt thereof to prepare a medicament for delivering an oligonucleotide to cells that express platelet-derived growth factor receptor (PDGFR).

67. The use of a compound as described in any one of claims 1-24 or a pharmaceutically acceptable salt thereof to prepare a medicament for treating a disease involving PDGFR expressing cells (e.g., liver fibrosis, non-alcoholic steatohepatitis (NASH), clear cell renal cell carcinoma, kidney fibrosis, or alcoholic steatohepatitis (ASH)).

68. The method, compound of use of any one of claims 56-67, which comprises the combined use of a first compound as described in any one of claims 1-24, wherein each R² is independently an oligonucleotide, with a second compound as described in any one of claims 50-53.


SEQUENCE LISTING (23 sequences)

SEQ ID NO: 1 - protein, 8 aa, synthetic construct
  DISULFID 1..8: note = Intrachain disulfide bond
CSRNLIDC

SEQ ID NO: 2 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 3 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 4 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 5 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 6 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 7 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 8 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 9 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 10 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 11 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 12 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 13 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 14 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 15 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 16 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
ggaaagactgttccaaaaa

SEQ ID NO: 17 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tttttggaacagtctttcctt

SEQ ID NO: 18 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
gagacacatgggtgctatt

SEQ ID NO: 19 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
aatagcacccatgtgtctcag

SEQ ID NO: 20 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide
gattatgtccggttatgta

SEQ ID NO: 21 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tacataaccggacataatcat

SEQ ID NO: 22 - protein, 7 aa, synthetic construct
  DISULFID 1..7: note = Intrachain disulfide bond
CSNLIDC

SEQ ID NO: 23 - protein, 8 aa, synthetic construct
  DISULFID 1..8: note = Intrachain disulfide bond
CIDNLSRC